SEQUENCE LISTING (6 sequences)

SEQ ID NO: 1 - protein, 4 aa, Homo sapiens
DAHK

SEQ ID NO: 2 - protein, 8 aa, Artificial Sequence
  Description of Artificial Sequencemetal
DAHGGHAX

SEQ ID NO: 3 - protein, 12 aa, Homo sapiens
DAHKSEVAHRFK

SEQ ID NO: 4 - protein, 11 aa, Homo sapiens
AHKSEVAHRFK

SEQ ID NO: 5 - protein, 10 aa, Homo sapiens
HKSEVAHRFK

SEQ ID NO: 6 - protein, 12 aa, Homo sapiens
  MOD_RES (1): ACETYLATION
DAHKSEVAHRFK

(19) United States
(12) Patent Application Publication
(10) Pub. No.: US 2003/0158111 A1
(43) Pub. Date: Aug. 21, 2003

(54) Methods and products for oral care

(76) Inventor: David Bar-Or, Englewood, CO (US)

Correspondence Address: SHERIDAN ROSS PC, 1560 BROADWAY, SUITE 1200, DENVER, CO, 80202

(21) Appl. No.: 10/300,664

(22) Filed: Nov. 19, 2002

Related U.S. Application Data
(63) Continuation-in-part of application No. 10/186,168, filed on Jun. 27, 2002.
Application No. 10/186,168 is a continuation-in-part of application No. 10/076,071, filed on Feb. 13, 2002.
Application No. 10/186,168 is a continuation-in-part of application No. 09/678,202, filed on Sep. 29, 2000.
(60) Provisional application No. 60/268,558, filed on Feb. 13, 2001.
(60) Provisional application No. 60/281,648, filed on Apr. 4, 2001.
(60) Provisional application No. 60/283,507, filed on Apr. 11, 2001.
(60) Provisional application No. 60/157,404, filed on Oct. 1, 1999.
(60) Provisional application No. 60/211,078, filed on Jun. 13, 2000.
(60) Provisional application No. 60/331,665, filed on Nov. 20, 2001.
(60) Provisional application No. 60/360,736, filed on Feb. 27, 2002.

(51) Int. Cl. A61K 38/16; A61K 38/10; A61K 38/08; A61K 38/06; A61K 7/28

(52) U.S. Cl. 514/12; 514/13; 514/14; 514/15; 514/16; 514/17; 514/18; 424/50

(57) ABSTRACT

The invention provides methods, oral care products and kits for treating mouth tissues. In particular, the invention provides methods, oral care products and kits which comprise certain metal-binding peptides, peptide derivatives and peptide dimers that can reduce inflammation of tissues of the mouth and can reduce the damage done by reactive oxygen species (ROS) to such tissues.

### CROSS REFERENCE TO RELATED APPLICATIONS

This application is a continuation-in-part (CIP) ofpending U.S. application Ser. No. 10/186,168, filed Jun. 27, 2002, which was a CIP of pending U.S. application Ser. No. 10/076,071, filed Feb. 13, 2002 (which claims benefit of provisional applications No. 60/268,558, filed Feb. 13, 2001, ______ (formerly 09/816,679), filed Mar. 22, 2001, 60/281,648, filed Apr. 4, 2001, and 60/283,507, filed Apr. 11, 2001), which was a CIP of pending application 09/678,202, filed Sep. 29, 2000 (which claims benefit of provisional applications No. 60/157,404, filed Oct. 1, 1999, and 60/211,078, filed Jun. 13, 2000). This application also claims benefit of provisional applications Nos. 60/331,665, filed Nov. 19, 2001, and 60/360,736, filed Feb. 27, 2002. The entire disclosures of all of the aforementioned applications are considered to be part of the disclosure of this application and are hereby incorporated by reference.

### FIELD OF THE INVENTION

The invention relates to methods, oral care products and kits for treating mouth tissues. In particular, the invention relates to methods, oral care products and kits which comprise certain metal-binding peptides, peptide derivatives and peptide dimers that can reduce inflammation of the tissues of the mouth and can reduce the damage done by reactive oxygen species (ROS) to such tissues.

### BACKGROUND

Reactive oxygen species (ROS) include free radicals (e.g., superoxide anion and hydroxyl, peroxyl, and alkoxyl radicals) and non-radical species (e.g., singlet oxygen and hydrogen peroxide). ROS are capable of causing extensive molecular, cellular and tissue damage, and they have been reported to play a major role in a variety of diseases and conditions. Indeed, ROS have been implicated in over 100 diseases and pathogenic conditions, and it has been speculated that ROS may constitute a common pathogenic mechanism involved in all human diseases. Stohs, J. Basic Clin. Physiol. Pharmacol., 6, 205-228 (1995). For reviews describing ROS, their formation, the mechanisms by which they cause molecular, cellular and tissue damage, and their involvement in numerous diseases and disorders, see, e.g., Manso, Rev. Port. Cardiol., 11, 997-999 (1992); Florence, Aust. NZJ Opthalmol., 23, 3-7 (1992); Stohs, J. Basic Clin. Physiol Pharmacol., 6, 205-228 (1995); Knight, Ann. Clin. Lab. Sci., 25, 111-121 (1995); Kerr et al., Heart & Lung, 25, 200-209 (1996); Roth, Acta Chir. Hung., 36, 302-305 (1997).

Metal ions, primarily transition metal ions, can cause the production and accumulation of ROS. In particular, copper and iron ions released from storage sites are one of the main causes of the production of ROS following injury, including ischemia/reperfusion injury and injury due to heat, cold, trauma, excess exercise, toxins, radiation, and infection. Roth, Acta Chir. Hung., 36, 302-305 (1997). Copper and iron ions, as well as other transition metal ions (e.g., vanadium, and chromium ions), have been reported to catalyze the production of ROS. See, e.g., Stohs, J. Basic Clin. Physiol. Pharmacol., 6, 205-228 (1995); Halliwell et al., Free Radicals In Biology And Medicine, pages 1-19 (Oxford University 1989); Marx et al.,Biochem. J, 236, 397-400(1985); Quinlan et al., J. Pharmaceutical Sci., 81, 611-614 (1992). Other transition metal ions (e.g., cadmium, mercury, and nickel ions) and other metal ions (e.g., arsenic and lead ions) have been reported to deplete some of the molecules of the natural antioxidant defense system, thereby causing an increased accumulation of ROS. See, e.g., Stohs, J. Basic Clin. Physiol. Pharmacol., 6, 205-228 (1995). Although it has been reported that free copper ions bind nonspecifically to the amino groups of essentially any protein (Gutteridge et al., Biochim. Biophys. Acta, 759, 38-41 (1983)), copper ions bound to proteins can still cause the production of ROS which damage at least the protein to which the copper ions are bound. See, e.g., Gutteridge et al., Biochim. Biophys. Acta, 759, 38-41 (1983); Marx et al., Biochem. J., 236, 397-400 (1985); Quinlan et al., J. Pharmaceutical Sci., 81, 611-614 (1992).

ROS may be present in the mouth for a variety of reasons. For instance, ROS are present in the mouth as a result of the use of tobacco products, exposure to environmental agents, exposure to radiation, and the use of oral care products comprising tooth whitening agents that liberate active oxygen or hydrogen peroxide. See, e.g., U.S. Pat. Nos. 5,906,811, 6,228,347, and 6,270,781. ROS may also be present in the mouth as a result of diseases and conditions that involve inflammation and/or infection, including gingivitis, periodontitis, injuries, surgeries, tooth extractions, cold sores, canker sores and ulcers. See, e.g, U.S. Pat. Nos. 6,228,347 and 6,270,781. Finally, although the normal pH of saliva is 7.2, acidic conditions often are present in the mouth, e.g., as a result of the breakdown of foods, especially carbohydrates. See, e.g., U.S. Pat. No. 6,177,097. Acidic conditions promote the release of copper ions from proteins to which they are bound and, as discussed above, free copper ions can cause the production of ROS. The ROS present in the mouth can cause damage to the tissues of the mouth. For instance, in inflammatory periodontal diseases, ROS and elevated levels of free iron and copper ions have been found in periodontal pockets, suggesting a significant role for ROS in periodontal tissue destruction. See, e.g., Waddington et al., Oral Dis., 6: 138-151 (2000).

Albumin has been characterized as an extracellular antioxidant. See, e.g., Halliwell and Gutteridge, Arch. Biochem. Biophys., 280, 1-8 (1990); Das et al., Methods Enzymol., 233, 601-610 (1994); Stohs, J. Basic Clin. Physiol. Pharmacol., 6, 205-228 (1995); Dunphy et al., Am. J Physiol., 276, H1591-H1598 (1999)). The antioxidant character of albumin has been attributed to several of albumin's many physiological functions, including albumin's ability to bind metals (particularly copper ions), to bind fatty acids, to bind and transport steroids, to bind and transport bilirubin, to scavenge HOCl, and others. See, e.g., Halliwell and Gutteridge, Arch. Biochem. Biophys., 280, 1-8 (1990); Halliwell and Gutteridge, Arch. Biochem. Biophys., 246, 501-514 (1986); Stohs, J. Basic Clin. Physiol. Pharmacol., 6, 205-228 (1995); Dunphy et al., Am. J. Physiol., 276, H1591-H1598 (1999)). Albumin contains several metal binding sites, including one at the N-terminus. The N-terminal metal-binding sites of several albumins, including human, rat and bovine serum albumins, exhibit high-affinity for Cu(II) and Ni(II), and the amino acids involved in the high-affinity binding of these metal ions have been identified. See Laussac et al., Biochem., 23, 2832-2838 (1984); Predki et al., Biochem. J., 287, 211-215 (1992); Masuoka et al., J. Biol. Chem., 268, 21533-21537 (1993). It has been reported that copper bound to albumin at metal binding sites other than the high-affinity N-terminal site produce free radicals which causes extensive damage to albumin at sites dictated by the location of the “loose” metal binding sites, resulting in the characterization of albumin as a “sacrificial antioxidant.” See Marx et al., Biochem. J., 236, 397-400 (1985); Halliwell et al., Free Radicals In Biology And Medicine, pages 1-19 (Oxford University 1989); Halliwell and Gutteridge, Arch. Biochem. Biophys., 280, 1-8 (1990); Quinlan et al., J. Pharmaceutical Sci., 81, 611-614 (1992).

Despite the foregoing, attempts to use albumin as a treatment for cerebral ischemia have shown mixed results. It has been reported that albumin is, and is not, neuroprotective in animal models of cerebral ischemia. Compare Huh et al., Brain Res., 804, 105-113 (1998) and Remmers et al., Brain Res., 827, 237-242 (1999), with Little et al., Neurosurgery, 9, 552-558 (1981) and Beaulieu et al., J. Cereb. Blood Flow. Metab., 18, 1022-1031 (1998).

Mixed results have also been obtained using albumin in cardioplegia solutions for the preservation of excised hearts. As reported in Dunphy et al., Am. J. Physiol., 276, H1591-H1598 (1999), the addition of albumin to a standard cardioplegia solution for the preservation of excised hearts did not improve the functioning of hearts perfused with the solution for twenty-four hours. Hearts did demonstrate improved functioning when perfused with a cardioplegia solution containing albumin and several enhancers (insulin, ATP, corticosterone, and pyruvic acid). This was a synergistic effect, since the enhancers alone, as well as the albumin alone, did not significantly improve heart function. An earlier report of improved heart function using cardioplegia solutions containing albumin was also attributed to synergism between enhancers and albumin. See the final paragraph of Dunphy et al., Am. J Physiol., 276, H1591-H1598 (1999) and Hisatomi et al., Transplantation, 52, 754-755 (1991), cited therein. In another study, hearts perfused with a cardioplegia solution containing albumin increased reperfusion injury in a dose-related manner, as compared to a solution not containing albumin. Suzer et al., Pharmacol. Res., 37, 97-101 (1998). Based on their study and the studies of others, Suzer et al. concluded that albumin had not been shown to be effective for cardioprotection. They further noted that the use of albumin in cardioplegia solutions could be unsafe due to possible allergic reactions and the risks associated with the use of blood products.

Finally, although albumin has been characterized as an antioxidant, it has also been reported to enhance superoxide anion production by microglia (Si et al., GLIA, 21, 413-418 (1997)). This result led the authors to speculate that albumin leaking through the disrupted blood brain barrier in certain disorders potentiates the production of superoxide anion by microglia, and that this increased production of superoxide anion is responsible for the pathogenesis of neuronal damage in cerebral ischemia/reperfusion and some neurodegenerative diseases.

As noted above, the N-terminal metal-binding sites of several albumins exhibit high-affinity for Cu(II) and Ni(II). These sites have been studied extensively, and a general amino terminal Cu(II)- and Ni(II)-binding (ATCUN) motif has been identified. See, e.g., Harford and Sarkar, Acc. Chem. Res., 30, 123-130 (1997). The ATCUN motif can be defined as being present in a protein or peptide which has a free —NH₂ at the N-terminus, a histidine residue in the third position, and two intervening peptide nitrogens. See, e.g., Harford and Sarkar, Acc. Chem. Res., 30, 123-130 (1997). Thus, the ATCUN motif is provided by the peptide sequence Xaa Xaa His, where Xaa is any amino acid except proline. See, e.g., Harford and Sarkar, Acc. Chem. Res., 30, 123-130 (1997). The Cu(II) and Ni(II) are bound by four nitrogens provided by the three amino acids of the ATCUN motif (the nitrogen of the free —NH₂, the two peptide nitrogens, and an imidazole nitrogen of histidine) in a slightly distorted square planar configuration. See, e.g., Harford and Sarkar, Acc. Chem. Res., 30, 123-130 (1997). Side-chain groups of the three amino acids of which the ATCUN motif consists can be involved in the binding of the Cu(II) and Ni(lI), and amino acids near these three N-terminal amino acids may also have an influence on the binding of these metal ions. See, e.g., Harford and Sarkar, Acc. Chem. Res., 30, 123-130 (1997); Bal et al., Chem. Res. Toxicol., 10, 906-914 (1997). For instance, the sequence of the N-terminal metal-binding site of human serum albumin is Asp Ala His Lys [SEQ ID NO:1], and the free side-chain carboxyl of the N-terminal Asp and the Lys residue have been reported to be involved in the binding of Cu(II) and Ni(II), in addition to the four nitrogens provided by Asp Ala His. See Harford and Sarkar, Acc. Chem. Res., 30, 123-130 (1997); Laussac et al., Biochem., 23, 2832-2838 (1984); and Sadler et al., Eur. J Biochem., 220, 193-200 (1994).

The ATCUN motif has been found in other naturally-occurring proteins besides albumins, and non-naturally-occurring peptides and proteins comprising the ATCUN motif have been synthesized. See, e.g., Harford and Sarkar, Acc. Chem. Res., 30, 123-130(1997); Bal et al., Chem. Res. Toxicol., 10, 906-914 (1997); Mlynarz, et al., Speciation 98. Abstracts, http://www.jate.u-szeged.hu/˜spec98/abstr/mlynar.html. Cu(II) and Ni(II) complexes of ATCUN-containing peptides and proteins have been reported to exhibit superoxide dismutase (SOD) activity. See Cotelle et al., J. Inorg. Biochem., 46, 7-15 (1992); Ueda et al., J. Inorg. Biochem., 55, 123-130 (1994). Despite their reported SOD activity, these complexes still produce free radicals which damage DNA, proteins and other biomolecules. See Harford and Sarkar, Acc. Chem. Res., 30, 123-130 (1997); Bal et al., Chem. Res. Toxicol., 10, 915-21 (1997); Ueda et al., Free Radical Biol. Med., 18, 929-933 (1995);Ueda et al., J. Inorg. Biochem., 55, 123-130 (1994); Cotelle et al., J. Inorg. Biochem., 46, 7-15 (1992). As a consequence, it has been hypothesized that at least some of the adverse effects of copper and nickel in vivo (e.g., causing cancer and birth defects) are attributable to the binding of Cu(II) and Ni(II) to ATCUN-containing proteins which causes the production of damaging free radicals. See Harford and Sarkar, Acc. Chem. Res., 30, 123-130 (1997); Bal et al., Chem. Res. Toxicol., 10, 915-921 (1997); Cotelle et al., J. Inorg. Biochem., 46, 7-15 (1992). Cf. Koch et al., Chem. & Biol., 4, 549-60 (1997). The damaging effects produced by a Cu(II) complex of an ATCUN-containing peptide in combination with ascorbate have been exploited to kill cancer cells in vitro and to produce anti-tumor effects in vivo. See Harford and Sarkar, Acc. Chem. Res., 30, 123-130 (1997).

### SUMMARY OF THE INVENTION

The invention provides a method of treating a tissue of an animal's mouth. The method comprises contacting the tissue with an effective amount of a metal-binding peptide having the formula P₁-P₂ or a physiologically-acceptable salt thereof. The invention also provides an oral care product comprising the peptide P₁- P₂, or a physiologically-acceptable salt thereof, and a kit comprising the oral care product. Oral care products include oral care devices and oral care compositions.

In the formula P₁-P₂:

P₁ is Xaa₁ Xaa₂ His or Xaa, Xaa₂ His Xaa₃; and

P₂ is (Xaa₄)ₙ.

Xaa₁ is glycine (Gly), alanine (Ala), valine (Val), leucine (Leu), isoleucine (Ile), serine (Ser), threonine (Thr), aspartic acid (Asp), isoaspartic acid (i.e., Asp attached to Xaa₂ through its γ-carboxyl, hereinafter “isoAsp”), asparagine (Asn), glutamic acid (Glu), isoglutamic acid (i e., Glu attached to Xaa₂ through its γ-carboxyl, hereinafter “isoGlu”), glutamine (Gln), lysine (Lys), hydroxylysine (Hylys), histidine (His), arginine (Arg), ornithine (Orn), phenylalanine (Phe), tyrosine (Tyr), tryptophan (Trp), cysteine (Cys), methionine (Met) or α-hydroxymethylserine (HMS). In addition, Xaa₁ can be an amino acid which comprises a δ-amino group (e.g., Orn, Lys) having another amino acid or a peptide attached to it (e.g., Gly (δ)-Orn). Xaa₁ is preferably Asp, Glu, Arg, Thr, or HMS. More preferably, Xaa₁ is Asp or Glu. Most preferably Xaa₁ is Asp.

Xaa₂ is Gly, Ala, β-Ala, Val, Leu, Ile, Ser, Thr, Asp, Asn, Glu, Gln, Lys, Hylys, His, Arg, Om, Phe, Tyr, Trp, Cys, Met or HMS. Xaa₂ is preferably Gly, Ala, Val, Leu, Ile, Thr, Ser, Asn, Met, His or HMS. More preferably Xaa₂ is Ala, Val, Thr, Ser, Leu, or HMS. Even more preferably Xaa₂ is Ala, Thr, Leu, or HMS. Most preferably Xaa₂ is Ala.

Xaa₃ is Gly, Ala, Val, Lys, Arg, Orn, Asp, Glu, Asn, Gln, or Trp, preferably Lys.

Xaa₄ is any amino acid.

Finally, n is 0-100, preferably 0-10, more preferably 0-5, and most preferably 0.

One or more of the amino acids of P₁ and/or P₂ can be substituted with (a) a substituent that increases the lipophilicity of the peptide without altering the ability of P₁ to bind metal ions, (b) a substituent that protects the peptide from proteolytic enzymes without altering the ability of P₁ to bind metal ions, or (c) a substituent which is a non-peptide, metal-binding functional group that improves the ability of the peptide to bind metal ions.

The invention provides another method of treating a tissue of an animal's mouth. The method comprises contacting the tissue with an effective amount of a metal-binding peptide (MBP) having attached thereto a non-peptide, metal-binding functional group. The metal-binding peptide MBP may be any metal-binding peptide, not just P₁-P₂. The invention also provides an oral care product comprising the metal-binding peptide MBP having attached thereto a non-peptide, metal-binding functional group and a kit comprising the oral care product.

The invention provides yet another method of treating a tissue of an animal's mouth. The method comprises contacting the tissue with an effective arnount of a metal-binding peptide dimer of the formula P₃-L-P₃, wherein each P₃ may be the same or different and is a peptide which is capable of binding a metal ion, and L is a chemical group which connects the two P₃ peptides through their C-terminal amino acids. In a preferred embodiment, one or both of the two P₃ peptides is P₁. The invention also provides an oral care product comprising the metal-binding peptide dimer of the formula P₃-L-P₃ and a kit comprising the oral care product.

### BRIEF DESCRIPTION OF THE DRAWINGS

FIGS. 1A-D: Formulas of tetrapeptide Asp Ala His Lys [SEQ ID NO:1] showing points of possible substitution.

FIGS. 2A-B: Schematic diagrams of the synthesis of derivatives of the tetrapeptide Asp Ala His Lys [SEQ ID NO: 1] coming within the formula of FIG. 1C (FIG. 2A) and FIG. 1B (FIG. 2B).

FIG. 3A-B: Formulas of cyclohexane diamine derivatives.

FIGS. 3C-D: Schematic diagrams of syntheses of cyclohexane diamine derivatives of the tetrapeptide Asp Ala His Lys [SEQ ID NO: 1].

FIG. 4: Formula of a tetraacetic acid derivative of the tetrapeptide Asp Ala His Lys [SEQ ID NO:1].

FIG. 5: Formula of a bispyridylethylamine derivative of the tetrapeptide Asp Ala His Lys [SEQ ID NO:1].

FIGS. 6A-B: Formulas of mesoporphyrin IX with (FIG. 6B) and without (FIG. 6A) a bound metal ion M.

FIG. 6C: Formula of mesoporphyrin IX derivative of the tetrapeptide Asp Ala His Lys [SEQ ID NO: 1].

FIG. 7: Formulas of monosaccharides.

FIG. 8A-B: Graphs of absorbance at 532 nm (A532) versus incubation time in an assay for the production of hydroxyl radicals. In FIG. 8A, ▪=ascorbate only, ♦=copper and ascorbate, ▴=tetrapeptide (L-Asp L-Ala L-His L-Lys [SEQ ID NO: 1]), copper and ascorbate (tetrapeptide/copper ratio of 1:1), X=tetrapeptide, copper and ascorbate (tetrapeptide/copper ratio of 2:1). In FIG. 8B, ♦=copper and ascorbate and ▪=tetrapeptide, copper and ascorbate (tetrapeptide/copper ratio of 2:1).

FIG. 9: Graph of % inhibition versus concentration tetrapeptide (L-Asp L-Ala L-His L-Lys [SEQ ID NO: 1])-copper complex at a tetrapeptide/copper ratio of 1:1 in the xanthine oxidase assay for superoxide dismutase activity.

FIG. 10: Graph of absorbance at 560 nm (A560) versus time in an assay for superoxide radical production. In FIG. 10, ▪=ascorbate only, ♦=copper and ascorbate, Δ=tetrapeptide (L-Asp L-Ala L-His L-Lys [SEQ ID NO:1]), copper and ascorbate (tetrapeptide/copper ratio of 1:1), X=tetrapeptide, copper and ascorbate (tetrapeptide/copper ratio of 2:1).

FIG. 11: Gel after electrophoresis of DNA treated in various ways. Lane 1-17 μg/ml plasmid DNA (untreated control); Lane 2-17 μg/ml plasmid DNA and 50 μM CuCl₂; Lane 3-17 μg/ml plasmid DNA and 2.5 mM ascorbate; Lane 4-17 μg/ml plasmid DNA, 2.5 mM ascorbate, 50 μM CuCl₂, and 200 μM tetrapeptide (L-Asp L-Ala L-His L-Lys [SEQ ID NO:1]) (4:1 ratio tetrapeptide/copper); Lane 5-17 μg/ml plasmid DNA, 2.5 mM ascorbate, 50 μM CuCl₂, and 100 μM tetrapeptide (2:1 ratio tetrapeptide/copper); Lane 6-17 μg/ml plasmid DNA, 2.5 mM ascorbate, 50 μM CuCl₂, and 50 μM tetrapeptide (1:1 ratio tetrapeptide/copper); Lane 7-17 μg/ml plasmid DNA, 2.5 mM ascorbate, 50 μM CuCl₂, and 25 μM tetrapeptide (1:2 ratio tetrapeptide/copper); Lane 8-17 μg/ml plasmid DNA, 2.5 mM ascorbate, 50 μM CuCl₂, and 12.5 μM tetrapeptide (1:4 ratio tetrapeptide/copper); Lane 9-17 μg/ml plasmid DNA, 2.5 mM ascorbate, and 50 μM CuCl₂ (positive control); and Lane 10-DNA ladder.

FIG. 12A: Formulas of peptide dimers according to the invention.

FIGS. 12B-D: Diagrams illustrating the synthesis of peptide dimers according to the invention.

FIG. 13: TAE (tris acetic acid EDTA (ethylenediamine tetracetic acid)) agarose gel visualized with ethidium bromide showing attenuation of ROS-induced DNA double strand breaks in genomic DNA by D-Asp Ala His Lys. Lane 1- no treatment; Lane 2-CuCl₂, 50 μM; Lane 3-ascorbic acid, 100 μM; Lane 4- D-Asp Ala His Lys, 200 μM; Lane 5- CuCl₂, 10 μM+ascorbic acid, 50 μM; Lane 6-CuCl₂, 25 μM+ascorbic acid, 50 μM; Lane 7-CuCl₂, 50 μM+ascorbic acid, 50 μM; Lane 8- CuCl₂, 50 μM+ascorbic acid, 25 μM; Lane 9-CuCl₂, 50 μM+ascorbic acid, 100 μM; Lane 10-CuCl₂, 50 μM+ascorbic acid, 100 μM+D-Asp Ala His Lys, 50 μM; Lane 11-CuCl₂, 50 μM+ascorbic acid, 100 μM+D-Asp Ala His Lys, 100 μM; Lane 12-CuCl₂, 50 μM+ascorbic acid, 100 μM+D-Asp Ala His Lys, 200 μM.

FIG. 14: TAE agarose gel visualized with ethidium bromide showing attenuation of ROS-induced DNA double strand breaks in genomic DNA by D-Asp Ala His Lys. Lane 1-no treatment; Lane 2-CuCl₂, 50 μM; Lane 3- ascorbic acid, 500 μM; Lane 4-D-Asp Ala His Lys, 200 μM; Lane 5- CuCl₂, 10 μM+ascorbic acid, 500 μM; Lane 6-CuCl₂, 25 μM+ascorbic acid, 500 μM; Lane 7-CuCl₂, 50 μM+ascorbic acid, 500 μM; Lane 8-CuCl₂, 50 μM+ascorbic acid, 100 μM; Lane 9-CuCl₂, 50 μM+ascorbic acid, 250 μM; Lane 10-CuCl₂, 50 μM+ascorbic acid, 500 μM+D-Asp Ala His Lys, 50 μM; Lane 11-CuCl₂, 50 μM+ascorbic acid, 500 μM+D-Asp Ala His Lys, 100 μM; Lane 12-CuCl₂, 50 μM+ascorbic acid, 500 μM+D-Asp Ala His Lys, 200 μM.

FIG. 15: Southern Blot showing attenuation of ROS-induced DNA double strand breaks in telomere DNA by D-Asp Ala His Lys. Lane 1-no treatment; Lane 2-CuCl₂, 50 μM; Lane 3-ascorbic acid, 100 μM, Lane 4-D-Asp Ala His Lys, 200 μM; Lane 5-CuCl₂, 50 μM+ascorbic acid, 100 μM; Lane 6-CuCl₂, 50 μM+ascorbic acid, 100 μM+D-Asp Ala His Lys, 200 μM.

FIG. 16: Southern Blot showing attenuation of ROS-induced DNA double strand breaks in telomere DNA by D-Asp Ala His Lys. Lane 1-no treatment; Lane 2-CuCl₂, 50 μM; Lane 3-ascorbic acid, 500 μM; Lane 4-D-Asp Ala His Lys, 200 μM; Lane 5-CuCl₂, 50 μM+ascorbic acid, 100 μM; Lane 6-CuCl₂, 50 μM+ascorbic acid, 250 μM; Lane 7-CuCl₂, 50 μM+ascorbic acid, 500 μM; Lane 8-CuCl₂, 50 μM+ascorbic acid, 500 μM+D-Asp Ala His Lys, 50 μM; Lane 9-CuCl₂, 50 μM+ascorbic acid, 500 μM+D-Asp Ala His Lys, 100 μM.

FIGS. 17A-C: Graphs of interleukin-8 (IL-8) concentration versus various treatments of Jurkat cells (all treatments, except nil and copper-only treatments, contained ascorbic acid in addition to the other additives listed on the graphs).

FIG. 18: Graph showing the effect of copper and D-Asp D-Ala D-His D-Lys (d-DAHK), each alone or in combination, on interleukin 8 (IL-8) secretion from human umbilical vein endothelial cells (HUVEC). CTL=control. Values are mean±standard error.

FIG. 19A: Bar graph showing IL-8 concentrations for normal controls and gingivitis and peridontitis patients.

FIG. 19B: Bar graph showing tumor necrosis factor-α (TNFα) concentrations for normal controls and gingivitis and peridontitis patients.

FIG. 19C: Bar graph showing soluble tumor necrosis factor-α receptor (sTNFR75) concentrations for normal controls and gingivitis and peridontitis patients.

FIG. 19D: Bar graph showing IL-8 concentrations before (open bars), and at 0.5 hours (gray bars), 6 hours (black bars), 24 hours (horizontal lines in bars), and 72 hours (cross-hatched bars) after, application of Crest Whitestrips™ to the teeth of five normal controls (Patients D, O, H, K and Y).

FIG. 19E: Bar graph showing IL-8 concentrations before (open bars), and at 0.5 hours (gray bars) and 6 hours (black bars) after, application to the teeth of three normal controls (Patients R, BM and W) of Crest WhitestripsTM to which the tetrapeptide L-Asp L-Ala L-His L-Lys [SEQ ID NO:1] (also referred to as DAHK-1199) had been applied.

### DETAILED DESCRIPTION OF THE PRESENTLY-PREFERRED EMBODIMENTS

In the formula P₁- P₂, P₁ is Xaa₁ Xaa₂ His or is Xaa₃, Xaa₂ His Xaa₃, wherein Xaa₁, Xaa₂, and Xaa₃ are defined above. P₁ is a metal-binding peptide sequence that binds transition metal ions of Groups 1b-7b or 8 of the Periodic Table of elements (including V, Co, Cr, Mo, Mn, Ba, Zn, Hg, Cd, Au, Ag, Co, Fe, Ni, and Cu) and other metal ions (including As, Sb and Pb). The binding of metal ions by P₁ inhibits (i.e., reduces or prevents) the production of ROS and/or the accumulation of ROS by these metal ions and/or targets the damage done by ROS that may still be produced by the bound metal ions to the peptide itself. As a result, the damage that can be caused by ROS in the absence of the binding of the metal ions to P₁ is reduced. In particular, P₁ binds Cu(II), Ni(II), Co(II), and Mn(II) with high affinity. It should, therefore, be particularly effective in reducing the damage caused by the production and accumulation of ROS by copper and nickel. In addition, the binding of metal ions by P₁ inhibits inflammation (see Examples 10, 12 and 13) and protects against inhibition of metabolism and energy utilization by cells (see Example 11).

In P₁, Xaa₁ is most preferably Asp, Xaa₂ is most preferably Ala, and Xaa₃ is most preferably Lys (see above). Thus, the preferred sequences of P₁ are Asp Ala His and Asp Ala His Lys [SEQ ID NO:1]. Most preferably the sequence of P₁ is Asp Ala His Lys [SEQ ID NO:1]. Asp Ala His is the minimum sequence of the N-terminal metal-binding site of human serum albumin necessary for the high-affinity binding of Cu(II) and Ni(II), and Lys has been reported to contribute to the binding of these metal ions to this site. Also, Asp Ala His Lys [SEQ ID NO:1] has been found by mass spectometry to bind Fe(II). Other preferred sequences for P₁ include Thr Leu His (the N-terminal sequence of human α-fetoprotein), Arg Thr His (the N-terminal sequence of human sperm protamin HP2) and HMS HMS His (a synthetic peptide reported to form extremely stable complexes with copper; see Mlynarz et al., Speciation 98. Abstracts, http://www.jate.u-szeged.hu/spec98/abstr/mlynar.html., Apr. 4, 1998).

P₂ is (Xaa₄)ₙ, wherein Xaa₄ is any amino acid and n is 0-100. When n is large (n>about 20), the peptides will reduce the damage done by ROS extracellularly. Smaller peptides are better able to enter cells, and smaller peptides can, therefore, be used to reduce the damage done by ROS both intracellularly and extracellularly. Smaller peptides are also less subject to proteolysis. Therefore, in P₂, preferably n is 0-10, more preferably n is 0-5, and most preferably n is 0. Although P₂ may have any sequence, P₂ preferably comprises a sequence which (1) binds a transition metal, (2) enhances the ability of the peptide to penetrate cell membranes, or (3) otherwise stabilizes or enhances the performance of the peptide. P₂ together with P₁ may also be the N-terminal sequence of a protein having an N-terminal metal-binding site with high affinity for copper and nickel, such as human, rat or bovine serum albumin. In the case where n=100, the peptide would have the sequence of approximately domain 1 of these albumins.

The sequences of many peptides which comprise a binding site for transition metal ions are known. See, e.g., U.S. Pat. Nos. 4,022,888, 4,461,724, 4,665,054, 4,760,051, 4,767,753, 4,810,693, 4,877,770, 5,023,237, 5,059,588, 5,102,990, 5,118,665, 5,120,831, 5,135,913, 5,145,838, 5,164,367, 5,591,711, 5,177,061, 5,214,032, 5,252,559, 5,348,943, 5,443,816, 5,538,945, 5,550,183, 5,591,711, 5,690,905, 5,759,515, 5,861,139, 5,891,418, 5,928,955, and 6,017,888, PCT applications WO 94/26295, WO 99/57262 and WO 99/67284, European Patent,application 327263, Lappin et al., Inorg. Chem., 17, 1630-34 (1978), Bossu et al., Inorg. Chem., 17, 1634-40 (1978), Chakrabarti, Protein Eng., 4, 57-63 (1990), Adman, Advances In Protein Chemistry, 42, 145-97 (1991), Cotelle et al., J. Inorg. Biochem., 46, 7-15 (1992), Canters et al., FEBS, 325, 39-48 (1993), Regan, Annu. Rev. Biophys. Biomol. Struct., 22, 257-281 (1993), Ueda et al., J. Inorg. Biochem., 55, 123-30 (1994), Ueda et al., Free Radical Biol. Med., 18, 929-33 (1995), Regan, TIBS, 20, 280-85 (1995), Uedaet al., Chem. Pharm. Bull., 43, 359-61 (1995), Bal et al., Chem. Res. Toxicol., 10, 906-914 (1997), Bal et al., Chem. Res. Toxicol., 10, 915-21 (1997), Koch et al., Chem. Biol, 4, 549-60 (1997), Kowalik-Jankowska et al., J. Inorg. Biochem., 66, 193-96 (1997), Harford and Sarkar, Acc. Chem. Res., 30, 123-130 (1997), Prince et al., TIBS, 23, 197-98 (1998), Mlynarz, et al., Speciation 98: Abstracts, http://www.jate.u-szeged.hu/˜spec98/abstr/mlynar.html, and Aitken, Molec. Biotechnol., 12, 241-53 (1999), Whittal et al., Protein Science, 9, 332-343 (2000). P₂ may comprise the sequence of one or more of the metal-binding sites of these peptides.

When P₂ comprises a metal-binding site, it preferably has a sequence which includes a short spacer sequence between P₁ and the metal binding site of P₂, so that the metal-binding sites of P₁ and P₂ may potentially cooperatively bind metal ions (similar to a 2:1 peptide:metal complex; see Example 8). Preferably, the spacer sequence is composed of 1-5, preferably 1-3, neutral amino acids. Thus, the spacer sequence may be Gly, Gly Gly, Gly Ala Gly, Pro, Gly Pro Gly, etc.

In particular, when P₂ comprises a metal-binding site, it preferably comprises one of the following sequences: (Xaa₄)ₘ Xaa₅ Xaa₂ His Xaa₃ or (Xaa₄)ₘ Xaa₅ Xaa₂ His. Xaa₂, Xaa₃ and Xaa₄ are defined above, and m is 0-5, preferably 1-3. The Xaa₄ amino acid(s), if present, form(s) a short spacer sequence between P₁ and the metal binding site of P₂ so that the metal-binding sites of P₁ and P₂ may cooperatively bind metal ions, and Xaa₄ is preferably a neutral amino acid (see the previous paragraph). Xaa₅ is an amino acid which comprises a δ-amino group (preferably Orn or Lys, more preferably Orn) having the Xaa₄ amino acid(s), if present, or P₁ attached to it by means of the δ-amino group. See Harford and Sarkar, Acc. Chem. Res., 30, 123-130 (1997) and Shullenberger et al., J. Am. Chem. Soc., 115, 11038-11039 (1993) (as a result of this means of attachment, the α-amino group of Xaa₅ can still participate in binding metals by means of the ATCUN motif). Thus, for instance, P₁- P₂ could be Asp Ala His Gly Gly (δ)-Orn Ala His [SEQ ID NO:2].

In addition, P₂ may comprise one ofthe following sequences: [(Xaa₄)ₘ Xaa₅ Xaa₂ His Xaa₃]ᵣ, [(Xaa₄)ₘ Xaa₅ Xaa₂ His(Xaa₄)ₘ Xaa₅ Xaa₂ His Xaa₃](Xaa₄)ₘ Xaa₅ Xaa₂ His]ᵣ, and [(Xaa₄)ₘ Xaa₅ Xaa₂ His(Xaa₄)ₘ Xaa₅ Xaa₂ His Xaa₃]ᵣ, wherein Xaa₂, Xaa₃, Xaa₄, Xaa₅ and m are defined and described above, and r is 2-100. In this manner metal-binding polymers may be formed.

In another preferred embodiment, P₂ comprises a peptide sequence that can bind Cu(I). As discussed in more detail below, Cu(II) is converted to Cu(I) in the presence of ascorbic acid or other reducing agents, and the Cu(I) reacts with oxygen to produce ROS (see equations in Examples 8 and 9). P₁ can bind Cu(II) tightly and is very effective by itself in inhibiting the production of ROS by copper (see Examples). However, as can be seen from the equations in Examples 8 and 9, it would be desirable to also employ a P₂ which could bind Cu(I).

Peptide sequences which can bind Cu(I) are known in the art. See, e.g, Pickering et al., J. Am. Chem. Soc., 115, 9498-9505 (1993); Winge et al., in Bioinorganic Chemistry Of Copper, pages 110-123 (Karlin and Tyeklar, eds., Chapman & Hall, New York, N.Y., 1993); Koch et al., Chem & Biol., 4, 549-560 (1997); Cobine et al., in Copper Transport And Its Disorders, pages 153-164 (Leone and Mercer eds., Kluwer Academic/Plenum Publishers, New York, N.Y., 1999). These sequences include:

Met Xaa₄ Met,

Met Xaa₄ Xaa₄ Met,

Cys Cys,

Cys Xaa₄ Cys,

Cys Xaa₄ Xaa₄ Cys,

Met Xaa₄ Cys Xaa₄ Xaa₄ Cys,

Gly Met Xaa₄ Cys Xaa₄ Xaa₄ Cys [SEQ ID NO:7],

Gly Met Thr Cys Xaa₄ Xaa₄ Cys [SEQ ID NO:8], and

Gly Met Thr Cys Ala Asn Cys [SEQ ID NO:9],

wherein Xaa₄ is defined above. Glutathione (γy-Glu Cys Gly) is also known to bind Cu(I). Additional Cu(I)-binding peptide sequences can be identified using a metallopeptide combinatorial library as described in, e.g., PCT application WO 00/36136. Preferably, the Cu(I)-binding peptide comprises the sequence Cys Xaa₄ Xaa₄ Cys (e.g., Gly Met Xaa₄ Cys Xaa₄ Xaa₄ Cys [SEQ ID NO:7], more preferably Gly Met Thr Cys Xaa₄ Xaa₄ Cys [SEQ ID NO:8], most preferably Gly Met Thr Cys Ala Asn Cys [SEQ ID NO:9]).

To enhance the ability of the P₁-P₂ peptide to penetrate cell membranes, P₂ is preferably hydrophobic or an arginine oligomer (see Rouhi, Chem. & Eng. News, 49-50 (Jan. 15, 2001)). When P₂ is hydrophobic, it preferably contains 1-3 hydrophobic amino acids (e.g., Gly Gly), preferably D-amino acids. The arginine oligomer preferably contains 6-9 Arg residues, most preferably 6-9 D-Arg residues (see Rouhi, Chem. & Eng. News, 49-50 (Jan. 15, 2001). The use of a P₂ which is an arginine oligomer may be particularly desirable when P₁-P₂ is to be administered topically.

The amino acids of the peptide may be L-amino acids, D-amino acids, or a combination thereof. Preferably, at least one of the amino acids of P₁ is a D-amino acid (preferably Xaa₁ and/or His), except for β-Ala, when present. Most preferably, all of the amino acids of P₁, other than β-Ala, when present, are D-amino acids. Also, preferably about 50% of the amino acids of P₂ are D-amino acids, and most preferably all of the amino acids of P₂ are D-amino acids. D-amino acids are preferred because peptides containing D-amino acids are resistant to proteolytic enzymes, such as those that would be encountered in the mouths of animals (including humans). Also, the use of D-amino acids would not alter the ability of the peptide to bind metal ions, including the ability of the peptide to bind copper with high affinity.

The peptides of the invention may be made by methods well known in the art. For instance, the peptides, whether containing L-amino acids, D-amino acids, or a combination of L- and D-amino acids, may be synthesized by standard solid-phase peptide synthesis methods. Suitable techniques are well known in the art, and include those described in Merrifield, in Chem. Polypeptides, pp. 335-61 (Katsoyannis and Panayotis eds. 1973); Merrifield, J. Am. Chem. Soc., 85, 2149 (1963); Davis et al., Biochem. Int'l, 10, 394-414 (1985); Stewart and Young, Solid Phase Peptide Synthesis (1969); U.S. Pat. Nos. 3,941,763 and 5,786,335; Finn et al., in The Proteins, 3rd ed., vol. 2, pp. 105-253 (1976); and Erickson et al. in The Proteins, 3rd ed., vol. 2, pp. 257-527 (1976). See also, Polish Patent 315474 (synthesis of HMS-containing peptides) and Shullenberger et al., J. Am. Chem. Soc., 115, 1103811039 (1993) (synthesis of (δ)-Orn-containing peptides). Alternatively, the peptides may be synthesized by recombinant DNA techniques if they contain only L-amino acids. Recombinant DNA methods and suitable host cells, vectors and other reagents for use therein, are well known in the art. See, e.g., Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989).

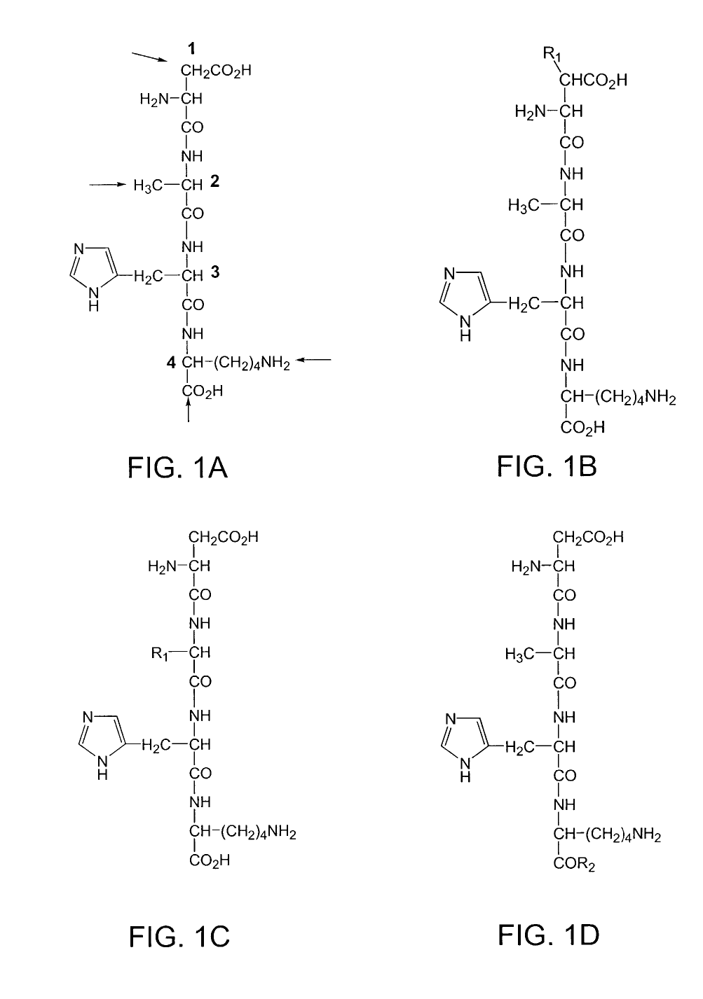

The invention further comprises derivatives ofthe peptide P₁-P₂, whether composed of L-amino acids, D-amino acids, or a combination of L- and D-amino acids, which are more resistant to proteolytic enzymes, more lipid soluble (to allow the peptides to more readily penetrate cell membranes), or both. As illustrated in FIG. 1A, P₁ can be modified in the regions indicated by the arrows without altering the metal binding function of P₁. In particular, P₁ can be substituted at carbons 1 or 2 with R₁, and the terminal —COOH of P₁ can be substituted with protecting group R₂ (FIGS. 1B-D). P₂ can be modified in ways similar to those described for P₁ to make P₂ more resistant to proteolytic enzymes, more lipid soluble, or both.

R₁ can be a straight-chain or branched-chain alkyl containing from 1 to 16 carbon atoms, and the term “alkyl” includes the R and S isomers. R₁ can also be an aryl or heteroaryl containing 1 or 2 rings. The term “aryl” means a compound containing at least one aromatic ring (e.g., phenyl, naphthyl, and diphenyl). The term “heteroaryl” means an aryl wherein at least one of the rings contains one or more atoms of S, N or O. These substitutions do not substantially decrease the ability of P₁ to bind metal ions. In particular, the ability of P₁ to bind copper with high affinity is not decreased by these substitutions. For instance, some of the substituents, such as a n-butyl attached to carbon 2 (see FIG. 1C, R₁ is n-butyl) should increase the affinity of the peptide for metal ions, such as copper, due to the inductive effect of the alkyl group. Substitution of carbon 2 (FIG. 1C) with an aryl, heteroaryl, or a long chain alkyl (about 6-16 carbon atoms) should enhance transport of the peptide across lipid membranes.

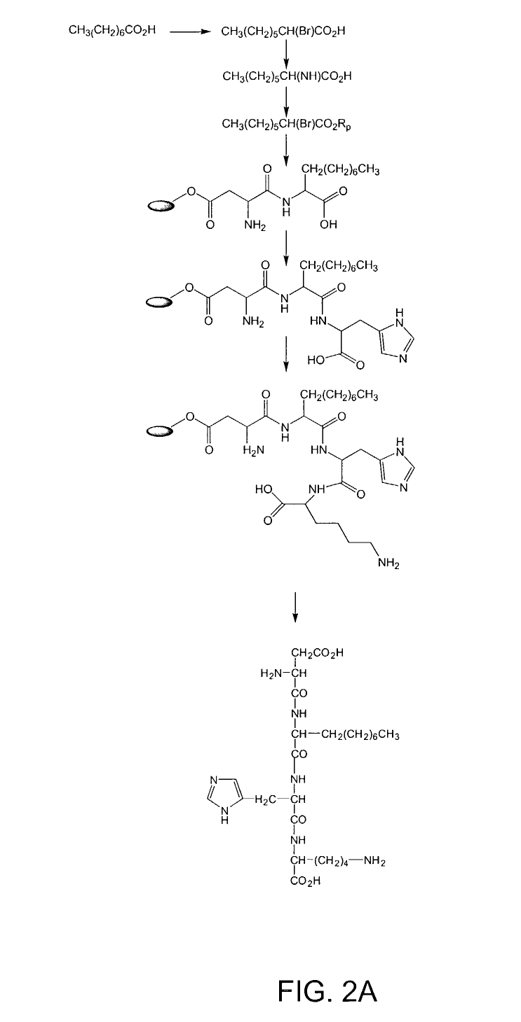

As noted above, methods of synthesizing peptides by solid phase synthesis are well known. These methods can be modified to prepare the derivatives shown in FIGS. 1B-C. For example, the derivative of Pl illustrated in FIG. 1C, wherein R₁ is octyl, can be prepared as illustrated in FIG. 2A. In FIG. 2A, the elliptical element represents the polymer resin and Rₚ is a standard carboxyl protecting group. As illustrated in FIG. 2A, octanoic acid (freshly distilled) is treated with dry bromine followed by phosphorus trichloride. The mixture is heated to about 100° C. and kept at that temperature for 4 hours. α-Bromooctanoic acid is obtained as a colorless liquid upon distillation. Anination of the bromoacid is achieved by allowing the acid and an ammonia solution to stand at 40-50° C. for 30 hours. The octyl derivative of the amino acid is obtained by removing ammonium bromide with methanol washes. Classical resolution methods give the desired optically-pure D-form. Other derivatives wherein R₁ is an alkyl, aryl or heteroaryl can be prepared in the manner illustrated in FIG. 2A.

In addition, the derivative of P₁ illustrated in FIG. 1B, wherein R₁ is phenyl, can be prepared as illustrated in FIG. 2B. In FIG. 2B, Polymer is the resin, t-Bu is t-butyl, and Bz is benzyl. Other derivatives wherein R₁ is an alkyl, aryl or heteroaryl can be prepared in the manner illustrated in FIG. 2B.

R₂ can be —NH₂, —NHR₁, —N(R₁)₂, —OR₁, or R₁ (see FIG. 1D), wherein R₁ is defined above. These derivatives can be prepared as the last step of a solid-phase peptide synthesis before the peptide is removed from the resin by methods well known in the art. Substitutions with R₂ do not substantially decrease the ability of P₁ to bind metal ions.

In addition, P₁ and P₂ can be substituted with non-peptide functional groups that bind metal ions. These metal-binding functional groups can be attached to one or more pendent groups ofthe peptide, and the resulting peptide derivatives will possess one or more sites that are capable of binding metal ions, in addition to the binding site provided by P₁ and, optionally, the binding site provided by P₂. As a consequence, the ability of such peptide derivatives to bind metal ions is improved as compared to the corresponding unmodified peptide. For instance, the peptide derivative can bind two of the same type of metal ion instead of one (e.g., two Cu(II)), the peptide derivative can bind two different metal ions instead of one type of metal ion (e.g., one Cu(II) and one Fe(III)), or the peptide derivative can bind one metal ion better (e.g., with greater affinity) than the corresponding unmodified peptide.

Metal-binding functional groups include polyamines (e.g., diamines, triamines, etc.). Suitable diamines include 1,2-alkyldiamines, preferably alkyl diamines wherein the alkyl contains 2-10 carbon atoms (e.g., H₂N—(CH₂)ₙ—NH₂, wherein n=2-10). Suitable diamines also include 1,2-aryldiamines, preferably benzene diamines (e.g., 1,2-diaminobenzene). Suitable diamines further include 1,2-cyclic alkane diamines. “Cyclic alkanes” are compounds containing 1-3 rings, each containing 5-7 carbon atoms. Preferably the cyclic alkane diamine is 1,2-diaminocylcohexane (cyclohexane diamine).

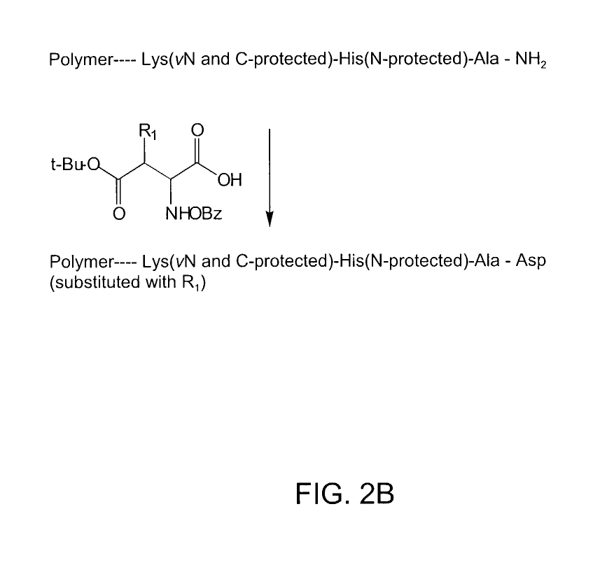

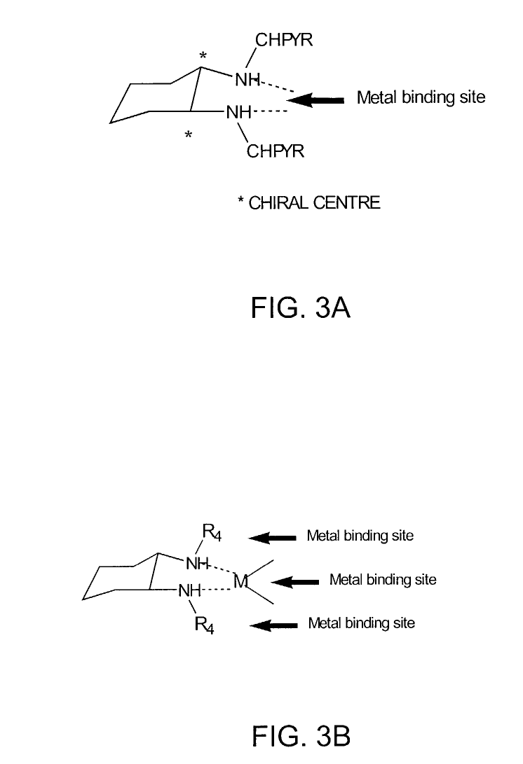

A particularly preferred diamine is 1,2-diaminocyclohexane (FIGS. 3A-B). Previous studies carried out by Rao & P. Williams (J Chromatography A, 693, 633 (1995)) have shown that a cyclohexane diamine derivative (FIG. 3A, where PYR is pyridine) binds to a variety of metal ions. The resulting metal chelator has been successfully used to resolve amino acids and peptides, showing that the molecule has a very high affinity for α-amino acids, forming a very stable coordination complex, which is unique in many respects. 1,2-Diaminocyclohexane possesses a reactive amino functional group to which a peptide of the invention can be attached. See FIG. 3B, where M is a metal ion and at least one R₄ is -alkyl-CO-peptide, -aryl-CO-peptide, -aryl-alkyl-CO-peptide, or -alkyl-aryl-CO-peptide (see also FIGS. 3C-D). The other R₄ may be the same or may be -alkyl-COOH, -aryl-COOH, -aryl-alkyl-COOH, or alkyl-aryl-COOH. Derivatives of the type shown in FIG. 3B will have several metal-binding sites and can, therefore, be expected to bind metal ions more readily than the unsubstituted peptide. Further, due to the presence of the cyclohexane functionality, the compound will possess lipid-like characteristic which will aid its transport across lipid membranes.

Cyclohexane diamine derivatives of the peptides of the invention can be prepared by 5 two distinct routes. The first involves initial condensation with an aldehyde followed by reduction (see FIG. 3C; in FIG. 3C Bz is benzyl). A number of aldehydes (alkyl and aryl) react readily with cyclohexane diamine at room temperature, forming an oxime. The oxime can be reduced with sodium borohydride under anaerobic conditions to give the diacid derivative. The carboxyl moieties are then reacted with the free amino groups present in carboxy-protected P₁ to give the cyclohexane diamine derivative of the peptide. The second route is a direct alkylation process which is illustrated in FIG. 3D. For example, cyclohexane diamine is treated with bromoacetic acid to give the diacetic acid derivative. The carboxyl moieties are then reacted with the free amino groups present in carboxy-protected P₁ to give the derivative. In FIG. 3D, R₅ is H or another peptide. When R₅ is H, the derivative can be further reacted to produce typical carboxylic acid derivatives, such as esters, by methods well known in the art. Metal binding experiments have indicated that the presence or absence of this group does not have a bearing on the metal binding capacity of the whole molecule. However, these groups would either make the molecule hydrophobic or hydrophilic, depending upon the substituent, and this may, in turn, have an effect on delivery of the molecule across membranes. These two synthetic routes will work for the synthesis of diamine peptide derivatives using the other diamines described above.

Additional suitable polyamines and polyamine derivatives and methods of attaching them to peptides are described in U.S. Pat. Nos. 5,101,041 and 5,650,134, the complete disclosures of which are incorporated herein by reference. Other polyamine chelators suitable for attachment to peptides are known. See, e g, U.S. Pat. Nos. 5,422,096, 5,527,522, 5,628,982, 5,874,573, and 5,906,996 and PCT applications WO 97/44313, WO 97/49409, and WO 99/39706.

It is well known that vicinal diacids bind to metal ions, and the affinity for copper is particularly high. It is therefore envisaged that a peptide having a vicinal diacid functional group will be extremely effective in metal binding. Suitable vicinal diacids include any 1,2-alkyldiacid, such as diacetic acid (succinic acid), and any 1,2-aryldiacid.

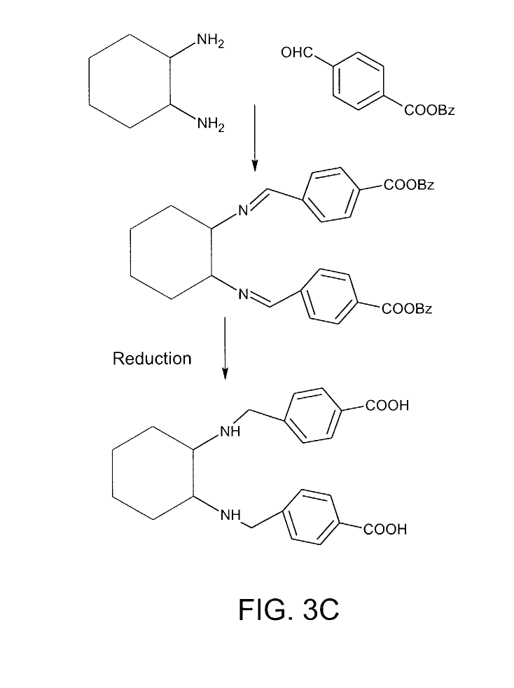

The amino groups of the peptide can be reacted with diacetic acid to produce a diacid derivative (see FIG. 4). This can be conveniently accomplished by reacting the amino groups of the resin-bound peptide with a halogenated acetic acid (e.g., bromoacetic acid or chloroacetic acid) or a halogenated acetic acid derivative (e.g., benzyloxy ester). Solid phase synthetic procedures enable removal of unreacted materials by washing with solvent. The final product is released from the resin by hydrolytic cleavage. Other diacid derivatives of the peptides of the invention can be made in the same manner.

Polyaminopolycarboxylic acids are known to bind metals, such as copper and iron. Suitable polyaminopolycarboxylic acids for making derivatives of the peptides of the invention and methods of attaching them to peptides are described in U.S. Pat. Nos. 5,807,535 and 5,650,134, and PCT application WO 93/23425, the complete disclosures of which are incorporated herein by reference. See also, U.S. Pat. No. 5,739,395.

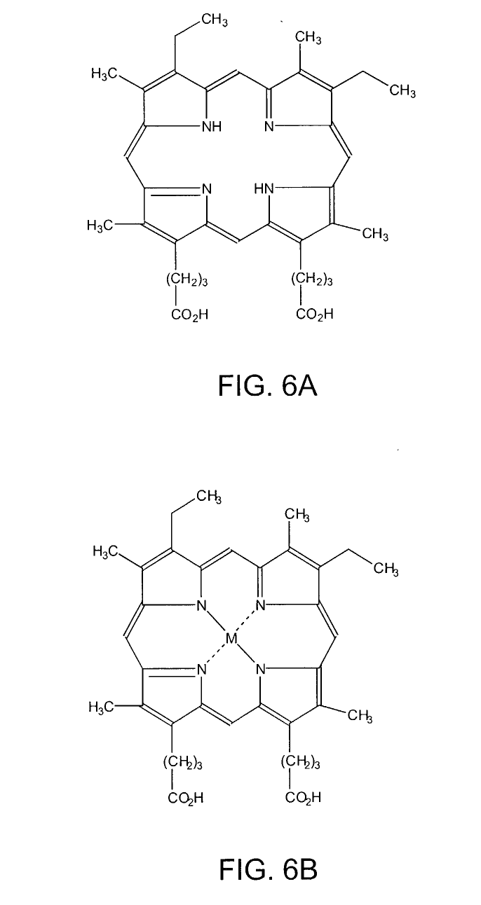

Vicinal polyhydroxyl derivatives are also included in the invention. Suitable vicinal polyhydroxyls include monosaccharides and polysaccharides (i. e., disaccharide, trisaccharide, etc.). Presently preferred are monosaccharides. See FIG. 7. The monosaccharides fall into two major categories—furanoses and pyranoses. One of the prime examples of a furanose ring system is glucose. The hydroxyl groups of glucose can be protected as benzyl or labile t-butyloxy functional groups, while leaving the aldehyde free to react with an amine group (e.g., that of lysine) of the tetrapeptide. Mild reduction/hydrolysis produces the monosaccharide peptide derivative. Other monosaccharide peptide derivatives can be prepared in this manner.

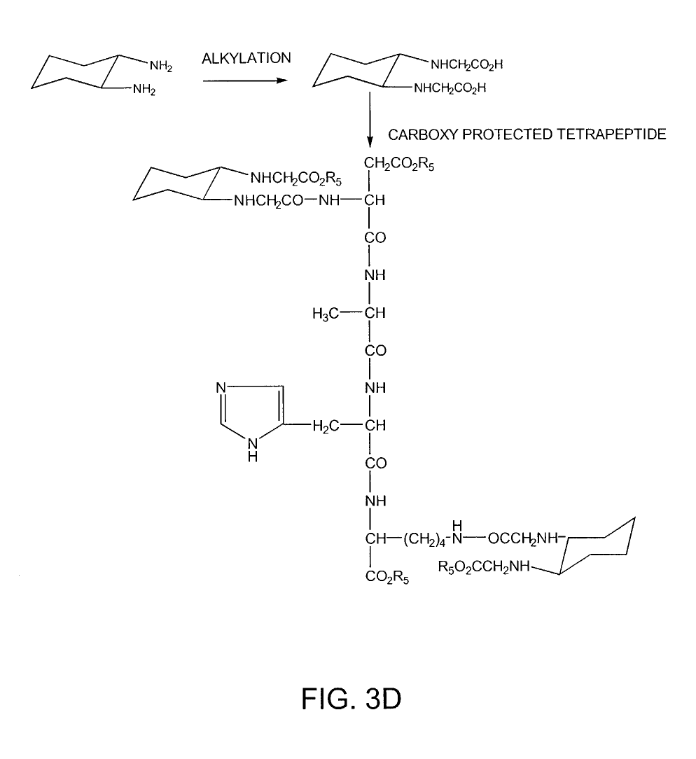

Bispyridylethylamine derivatives are known to form strong complexes with divalent metal ions. When attached to the peptide, this functional group would provide additional chelating sites for metal ions, including copper. The bispyridylethyl derivative of the tetrapeptide Asp Ala His Lys [SEQ ID NO:1] is shown in FIG. 5. It is anticipated that the metal-binding capacity of this tetrapeptide derivative will be increased by at least three-fold as compared to the underivatized peptide. The preparation of this bispyridylethylamine derivative shares some similarities with the synthesis of diacid derivatives. The two amino groups of the tetrapeptide (one at Asp and the other at Lys) are reacted with 2-bromoethylpyridine to give the tetra-substituted peptide derivative. The reaction is accomplished by reacting the resin-bound tetrapeptide with the bromoethylpyridine, followed by cleavage of the product from the resin.

Phenanthroline is another heterocyclic compound capable of binding divalent metal ions. Phenanthroline derivatives of the peptides can be synthesized in the same manner as for the bispyridylethylamine derivatives.

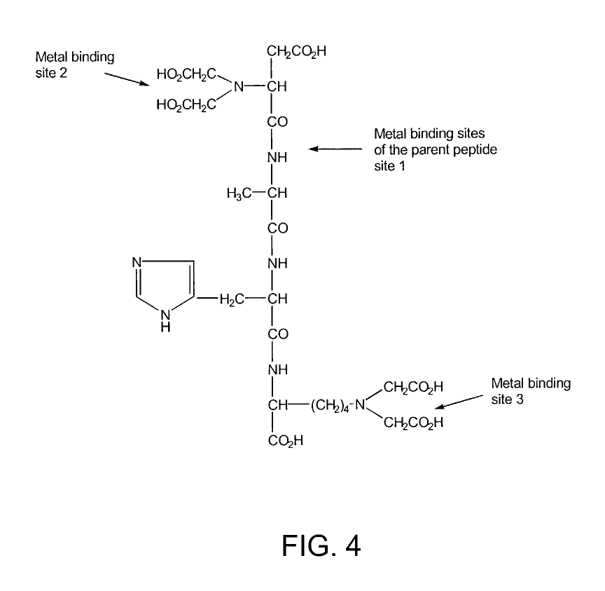

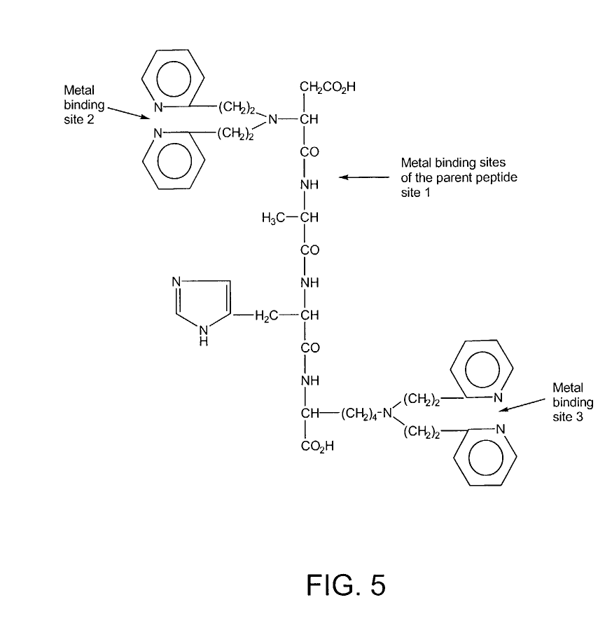

Porphyrins are a group of compounds found in all living matter and contain a tetrapyrrolic macrocycle capable of binding to metals. Heme, chlorophyll and corrins are prime examples of this class of compounds containing iron, magnesium and cobalt, respectively. Mesoporphyrin IX (FIG. 6A-B, where M is a metal ion) is derived from heme and has been observed to possess specific affinity for copper. Addition of this structure to a peptide of the invention would produce a porphyrin-peptide derivative possessing several sites for binding of copper (see FIG. 6C). In addition to their roles in metal binding, the imidazole residues at positions 3 and 3′ of the tetrapeptide shown in FIG. 6C may provide a binding site for metals other than copper, thereby stabilizing the porphyrin-metal complex. In particular, cyanocobalamine (vitamin B-12) contains cobalt as the metal in the porphyrin nucleus, and the complex is stabilized by the imidazole groups. On the basis of this analogy it is anticipated that the porphyrin-tetrapeptide derivative would bind cobalt (or other metals) at normal physiological conditions in the prophyrin nucleus and that the complex would be stabilized by the His imidazole groups.

To prepare the porphyrin-peptide derivative shown in FIG. 6C, the carboxyl groups of mesoporphyrin IX can be activated and coupled with the amino groups of the peptide employing standard solid-phase peptide synthesis. Typically, the free amino group of the lysine residue of the resin-bound peptide can be coupled with carboxy activated porphyrin nucleus. The condensation product can be cleaved off the resin using standard methods. This method can be used to synthesize other porphyrin derivatives of peptides of the invention.

Other suitable porphyrins and macrocyclic chelators and methods of attaching them to peptides are described in U.S. Pat. Nos. 5,994,339 and 5,087,696, the complete disclosures of which are incorporated herein by reference. Other porphyrins and macrocyclic chelators that could be attached to peptides are known. See, e.g., U.S. Pat. Nos. 5,422,096, 5,527,522, 5,628,982, 5,637,311, 5,874,573, and 6,004,953, PCT applications WO 97/44313 and WO 99/39706.

A variety of additional metal chelators and methods of attaching them to proteins are described in U.S. Pat. No. 5,683,907, the complete disclosure of which is incorporated herein by reference.

Dithiocarbamates are known to bind metals, including iron. Suitable dithiocarbamates for making derivatives ofthe peptides ofthe invention are described in U.S. Pat. Nos. 5,380,747 and 5,922,761, the complete disclosures of which are incorporated herein by reference.

Hydroxypyridones are also known to be iron chelators. Suitable hydroxypyridones for making derivatives of the peptides of the invention are described in U.S. Pat. Nos. 4,912,118 and 5,104,865 and PCT application WO 98/54138, the complete disclosures of which are incorporated herein by reference.

Additional non-peptide metal chelators are known in the art or will be developed. Methods of attaching chemical compounds to proteins and peptides are well known in the art, and attaching non-peptide metal chelators to the peptides of the invention is within the skill in the art. See, e.g., those patents cited above describing such attachment methods.

As can be appreciated, the non-peptide, metal-binding functional groups could be attached to another metal-binding peptide (MBP) in the same manner as they are to peptide P₁-P₂. The resulting peptide derivatives would contain one or more metal-binding functional groups in addition to the metal-binding site of MBP. Preferably, MBP contains from 2-10, more preferably 3-5, amino acids. Preferably MBP contains one or more D-amino acids; most preferably all of the amino acids of MBP are D-amino acids. As described above, the sequences of many metal-binding peptides are known. These peptides and peptides comprising the metal-binding sites of these peptides can be prepared in the same ways as described above for peptide P₁-P₂. Derivatives of these peptides having one or more metal-binding functional group attached to the peptide can be prepared in the same ways as described above for derivatives of peptide P₁- P₂.

The invention also provides metal-binding peptide dimers of the formula:

P₃-L-P₃.

P₃ is any peptide capable of binding a metal ion, and each P₃ may be the same or different. Each P₃ preferably contains 2-10, more preferably 3-5, amino acids. As described above, metal-binding peptides are known, and each P₃ may comprise the sequence of one or more of the metal-binding sites of these peptides. Although each P₃ may be substituted as described above for P₁ and P₂, including with a non-peptide, metal-binding functional group, both P₃ peptides are preferably unsubstituted. P₃ may also comprise any amino acid sequence substituted with a non-peptide, metal-binding functional group as described above to provide the metal-binding capability of P₃. Preferably, each P₃ is an unsubstituted metal-binding peptide (i.e., an unsubstituted peptide comprising a peptide sequence which binds metal ions). Most preferably, one or both of the P₃ groups is P₁ (i.e., the dimers have the sequence P₃-L-P₁, P₁-L-P₃ or, most preferably, P₁-L-P₁). P₁ is defined above.

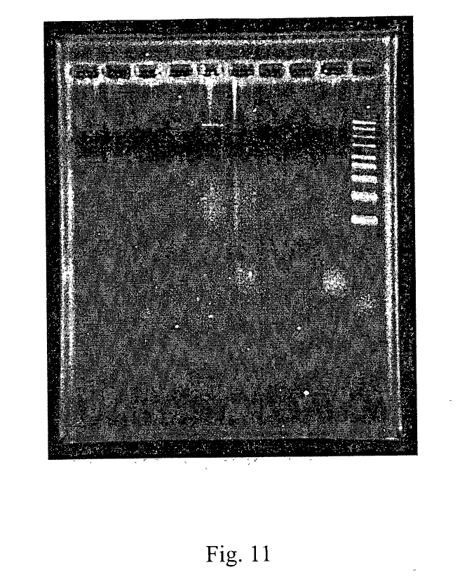

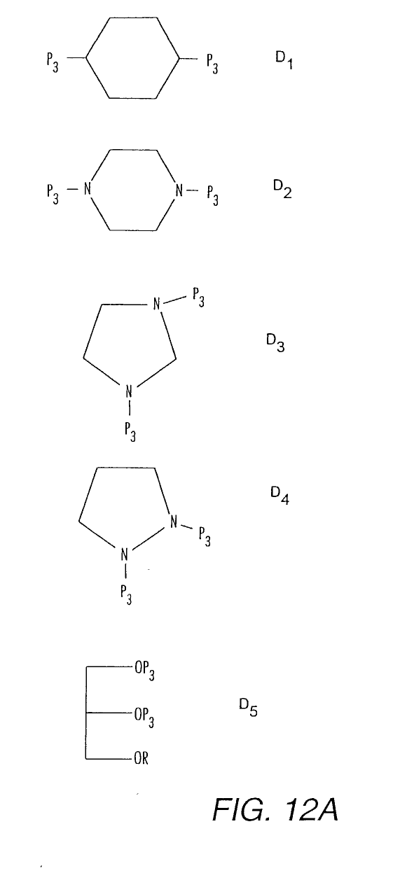

L is a linker which is attached to the C-terminal amino acid of each P₃. L may be any physiologically-acceptable chemical group which can connect the two P₃ peptides through their C-terminal amino acids. By “physiologically-acceptable” is meant that a peptide dimer containing the linker L is not toxic to an animal (including a human) or an organ to which the peptide dimer is administered as a result of the inclusion of the linker L in the peptide dimer. Preferably, L links the two P₃ groups so that they can cooperatively bind metal ions (similar to a 2:1 peptide:metal complex; see Example 8). L is also preferably neutral. Most preferably, L is a straight-chain or branched-chain alkane or alkene residue containing from 1-18, preferably from 2-8, carbon atoms (e.g., —CH₂—, —CH₂CH₂—, —CH₂CH₂CH₂—, —CH₂CH₂(CH₃)CH₂—, —CHCH—, etc.) or a cyclic alkane or alkene residue containing from 3-8, preferably from 5-6, carbon atoms (see FIG. 12A, compound D₁), preferably attached to a P₃ by means of an amide linkage. Such linkers are particularly preferred because they impart hydrophobicity to the peptide dimers. In another preferred embodiment, L is a nitrogen-containing heterocyclic alkane residue (see FIG. 12A, compounds D₂, D₃ and D₄), preferably a piperazide (see FIG. 12A, compound D₂). In another preferred embodiment L is a glyceryl ester (see FIG. 12A, compound D₅; in formula D₅, R is an alkyl or aryl containing, preferably containing 1-6 carbon atoms). Finally, L could be a metal-binding porphyrin (see FIG. 6C). These preferred linkers L will allow the two peptides P₃ to bind metal ions cooperatively and are biocompatible, and the peptide dimers containing these preferred linkers can be made easily and in large quantities. By “biocompatible” is meant that a peptide dimer containing the linker L does not produce any undesirable side-effects due to the linker L in an animal (including a human) to which the peptide dimer is administered.

Methods of synthesizing the peptide dimers are illustrated in FIGS. 12B-D. In general, the C-terminal amino acids (protected by methods and protecting groups well known in the art) of the two P₃ groups are attached to L, and the resulting amino acid dimers used in standard peptide synthetic methods to make the peptide dimers.

For instance, a peptide dimer, where each peptide has the sequence Asp Ala His Lys, [SEQ ID NO:1] can be synthesized by coupling protected lysines to a free diamine functional group, either as an acid chloride or by using standard coupling agents used in peptide synthesis (see FIGS. 12B-C). Many suitable diamines are available commercially or suitable diamines can be readily synthesized by methods known in the art.

For instance, the lysine dimer 2 (FIG. 12B) can be prepared as follows. To a stirred solution of 9-fluorenylmethyloxycarbonyl (Fmoc)- and t-benzyloxycarbonyl(Boc)- protected D-Lys (Fmoc-D-Lys(Boc)-OH) (20 mmole) in dry dimethylformamide (DMF; 100 mL; dry argon flushed) are added butane-1,4-diamine 1 and 2-(1H-benzotriazole-1-yl)-1,2,3,3-tetramethyluroniumtetrafluoroborate (TBTU; 0.5 mmole). The solution is stirred for 36 hours at room temperature. The bis-protected lysine 2 is isolated by flash chromatography over silica and elution with mixtures of ethyl acetate/methanol. The peptide dimer 3 is then prepared from the protected lysine dimer 2 employing classical peptide synthesis methodology (see FIG. 12B).

Another peptide dimer, where each peptide has the sequence Asp Ala His Lys [SEQ ID NO:1], can be synthesized as follows. First, a different protected lysine dimer 4 is synthesized by acylating the two amino centers of a piperazine 5 (see FIG. 12C; see also Chambrier et al., Proc. Natl. Acad Sci., 96, 10824-10829 (1999)). Then, the remainder of the amino acid residues are added employing standard peptide synthesis methodology to give the peptide dimer 6 (see FIG. 12C).

Peptide dimers, where each peptide has the sequence Asp Ala His Lys [SEQ ID NO: 1] and where L is a glyceryl ester, can be synthesized as follows. The 3-substituted propane-1,2-diols of formula 7 in FIG. 12D, wherein R is an alkyl or aryl, are commercially available. A lysine diester 8, wherein R is methyl, can be prepared as follows (see FIG. 12D). To a stirred solution of Fmoc-D-Lys(Boc)-OH (20 mmole) in dry toluene (100 mL; dry argon flushed) is added 3-methoxypropane-1,2-diol (200 mmole) and imidazole (15 mmole). The solution is stirred for 36 hours at room temperature. The solvent is removed in vacuo, and the residue is dissolved in ethyl acetate. This solution is washed with citric acid solution (2%), water, 0.5 N NaHCO₃ solution, and again with water; then the organic layer is dried over magnesium sulphate (removal of the solvent gives a pale yellow residue). The bis-protected lysine 8 is isolated by flash chromatography over silica and elution with mixtures of ethyl acetate/methanol. The peptide dimer 9 is then prepared from the protected lysine dimer 8 employing classical peptide synthesis methodology (see FIG. 12D).

The physiologically-acceptable salts ofthe metal-binding compounds are also included in the invention. Physiologically-acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, benzoic, salicylic, and the like) or bases (such as the hydroxide, carbonate or bicarbonate of a pharmaceutically-acceptable metal cation). The salts are prepared in a conventional manner, e g, by neutralizing the free base form of the compound with an acid.

The invention also provides oral care products comprising a metal-binding compound or compounds of the invention. Oral care products include oral care compositions and oral care devices.

Oral care compositions of the invention include washes, rinses, gargles, solutions, drops, emulsions, suspensions, liquids, pastes, gels, ointments, creams, sprays, powders, tablets, gums, lozenges, mints, films, patches, and tooth whitening compositions. Oral care compositions of the invention include compositions intended for use by consumers and patients and compositions intended for use by dental professionals (e.g., dental hygienists, dentists and oral surgeons).

The oral care compositions of the invention will comprise a metal-binding compound or compounds of the invention as active ingredient(s) in admixture with one or more pharmaceutically-acceptable carriers. Oral care compositions of the invention will generally comprise from about 0.001% to about 20% by weight of a metal-binding compound or a combination of metal-binding compounds of the invention. The oral care compositions of the invention may also comprise one or more other acceptable ingredients, including other active compounds and/or other ingredients conventionally used in oral care compositions. Each carrier and ingredient must be “acceptable” in the sense of being compatible with the other ingredients of the formulation and not injurious to the animal.

Suitable ingredients, including pharmaceutically-acceptable carriers, for use in oral care compositions, and methods of making and using oral care compositions, are well known in the art. See, e.g., U.S. Pat. Nos.4,847,283,5,032,384,5,043,183,5,180,578,5,198,220, 5,242,910, 5,286,479, 5,298,237, 5,328,682, 5,407,664, 5,466,437, 5,707,610, 5,709,873, 5,738,840, 5,817,295, 5,858,408, 5,876,701, 5,906,811, 5,932,193, 5,932,191, 5,951,966, 5,976,507,6,045,780,6,197,331,6,228,347,6,251,372, and6,350,438, PCT applications WO 95/32707, WO 96/08232 and WO 02/13775, and EP applications 471,396, the complete disclosure ofall ofwhich are incorporated herein by reference. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, pH-adjusting agents, and many others.

The water used in oral care compositions should preferably be of low ion content. It should also be free of organic impurities.

The alcohol must be nontoxic. Preferably the alcohol is ethanol. Ethanol is a solvent and also acts as an antibacterial agent and as an astringent.

Humectants suitable for use in oral care compositions include edible polyhydric alcohols such as glycerol, sorbitol, xylitol, butylene glycol, polyethylene glycol, propylene glycol, mannitol, and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacettes (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phospine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetylpyridinium chloride, cetyltrimethylammonium bromide, diisobuytylphenoxyethyldimethylbenzylammonium chloride, coconut alkyltrimetylammonium nitrite, cetylpyridinium fluoride). Certain cationic surfactants can also act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopol®), starch, copolymers of lactide and glycolide monomers (the copolymer having an average molecular weight of about 1,000- 120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphopsphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing.

Sweetening agents include sucrose, glucose, saccharin, dextrose, levulose, lactose, mannitol, sorbitol, fnictose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol.

Antibacterial and anti-plaque agents include triclosan, sanguinarine and sanguinaria, quaternary ammonium compounds, cetylpyridinium chloride, tetradecylpyridinium chloride and N-tetradecyl-4-ethylpyridinium chloride, benzalkonium chloride, bisquanides, chlorhexidine, chlorhexidine digluconate, hexetidine, octenidine, alexidine, halogenated bisphenolic compounds, 2,2′- methylenebis-(4-chloro-6-bromophenol), 5-chloro-2-(2,4-dichlorophenoxy)-phenol, salicylanilide, domiphen bromide, delmopinol, octapinol, other piperadino derivatives, nicin, zinc stannous ion agents, antibiotics (such as augimentin, amoxicillin, tetracycline, doxydcycline, minocycline, and metronidazole), analogs and salts of the foregoing, and mixtures of the foregoing.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

The pH of the oral compositions of the invention should not be acidic, since acidic conditions will lessen the effectiveness of the metal-binding compounds of the invention. Thus, the pH of the oral care compositions of the invention should be greater than about 6.5, preferably from about 7.0 to about 8.5, more preferably from about 7.2 to about 7.6. Thus, a pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), phosphate salts (such as potassium and sodium phosphate), and combinations of the foregoing as needed to achieve and maintain the desired pH.

The oral care compositions of the invention may further include one or more antioxidants, anti-inflammatory compounds, and/or metal-binding compounds in addition to the metal-binding compounds of the invention (which, as noted above, are anti-inflammatory and reduce the damage done by ROS, in addition to binding metal ions).

Suitable anti-inflammatory agents include ibuprofen, flurbiprofen, ketoprofen, aspirin, kertorolac, naproxen, indomethacin, piroxicam, meclofenamic acid, steroids, and mixtures of the foregoing.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others. See, e.g., Das et al., Methods Enzymol., 233, 601-610 (1994); Stohs, J. Basic Clin. Physiol. Pharmacol., 6, 205-228 (1995).

Suitable metal-binding compounds include metal-binding peptide and/or non-peptide chelators. Metal-binding peptides and non-peptide chelators are described above, and others are known in the art. For instance, a peptide P₁ (i.e., peptide P₁-P₂ wherein n=0 in the formula of P₂), which binds Cu(II) tightly, could be given in combination with a separate peptide suitable for binding Cu(I) (suitable Cu(I)-binding peptides are described above). As another example, a peptide P₁ could be given in combination with a separate peptide or non-peptide peptide chelator capable of binding iron. Suitable iron-binding peptides and non-peptide chelators are described above and others are known in the art (e.g., deferoxamine mesylate).

The oral care compositions of the invention may advantageously contain a protease inhibitor to prevent degradation of the metal-binding compounds of the invention and/or for an additional therapeutic effect (certain proteases are involved in inflammatory processes and others have been implicated in tissue breakdown in the mouth). Suitable protease inhibitors include metalloproteinase and serine protease inhibitors, such as those described in U.S. Pat. Nos. 6,403,633, 6,350,438, 6066673, 5,622,984, and 4,454,338, the complete disclosures of which are incorporated herein by reference.

Many other ingredients are known that may be incorporated into oral care compositions. These include suspending agents (such as a polysaccharide—see U.S. Pat. No. 5,466,437), polymeric compounds which can enhance the delivery of active ingredients (such as copolymers of polyvinylmethylether with maleic anhydride and those delivery enhancing polymers described in DE 942,643 and U.S. Pat. No. 5,466,437), materials which allow for a strong and continuing adherence of the oral care composition to the tissues of the mouth, thereby providing for a protracted topical therapeutic effect (such as natural gums, plant extracts, animal extracts (e g., gelatin), natural and synthetic polymers, and starch derivatives; see, e.g., U.S. Pat. Nos. 5,032,384, 5,298,237, and 5,466,437), oils, waxes, silicones, coloring agents (such as FD&C dyes), color change systems, preservatives (such as methylparaben, propylparaben, and sodium benzoate), opacifying agents (such as titanium dioxide), plant extracts, solubilizing agents (such as propylene glycol; see, e.g., U.S. Pat. No. 5,466,437), enzymes (such as dextranase and/or mutanase, amyloglucosidase, glucose oxidase with lactoperoxidase, and neuraminidases), synthetic or natural polymers, tooth whitening agents (such from about 0.1% to about 10% by weight of a peroxygen compound; see additional discussion oftooth whitening compositions below), an alkali metal bicarbonate (such as sodium bicarbonate (also known as baking soda), generally present at from about 0.01% to about 30% by weight), desensitizers (such as potassium salts (e.g., potassium nitrate, potassium citrate, potassium chloride, potassium tartrate, potassium bicarbonate, and potassium oxalate), and strontium salts), analgesics (such as lidocaine or benzocaine), anti-fungal agents, antiviral agents, etc.

The presence of a significant amount of copper and iron salts is preferably avoided. The presence of significant amounts of copper and iron ions in the oral care compositions could reduce the ability of the metal-binding compounds of the invention to bind copper and iron ions found in the mouth.

It will be appreciated that a wide variety of different oral care compositions can be prepared utilizing the above described ingredients and other ingredients known in the art or which will be developed. It is within the skill in the art to chose appropriate ingredients and combinations of ingredients and to determine an effective amount of the metal-binding compound(s) of the invention to include in a particular oral care composition, given the knowledge in the art and the guidance provided herein.

What follows are a few examples of oral care compositions into which a metal-binding compound or a combination of metal-binding compounds of the invention could be incorporated. It will be understood by those skilled in the art that additional types of oral care compositions and additional oral care compositions having different ingredients and/or different amounts of ingredients can be prepared utilizing the knowledge and skill in the art and the guidance provided herein.

Dentrifices include toothpastes, tooth gels, tooth powders and liquid dentrifices. Toothpastes and tooth gels generally include a dental abrasive, a surfactant, a thickening agent, a humectant, a flavoring agent, a sweetening agent, a coloring agent and water. Toothpastes and tooth gels may also include opacifying agents, anti-caries agents, anti-calculus agents, tooth whitening agents, and other optional ingredients. Typically, a toothpaste or tooth gel will contain from about 5% to about 70%, preferably from about 10% to about 50%, of an abrasive, from about 0.5% to about 10% of a surfactant, from about 0.1% to about 10% of a thickening agent, from about 10% to about 80% of a humectant, from about 0.04% to about 2% of a flavoring agent, from about 0.1% to about 3% of a sweetening agent, from about 0.01% to about 0.5% of a coloring agent, from about 0.05% to about 0.3% of an anti-caries agent, from about 0.1% to about 13% of an anti-calculus agent, and from about 2% to about 45% water. Tooth powders of course contain substantially all non-liquid components and typically contain from about 70% to about 99% abrasive. Liquid dentrifices may comprise water, ethanol, a humectant, a surfactant, a thickening agent, an abrasive (if an abrasive is included, a suspending agent (e.g., a high molecular weight polysaccharide) must be included; see U.S. Pat. No. 5,466,437), an antibacterial agent, an anti-caries agent, a flavoring agent and a sweetening agent. A typical liquid dentrifice will comprise from about 50% to about 85% water, from about 0.5% to about 20% ethanol, from about 10% to about 40% of a humectant, from about 0.5% to about 5% of a surfactant, from about 0.1% to about 10% of a thickening agent, and may contain from about 10% to about 20% of an abrasive, from about 0.3% to about 2% of a suspending agent, from about 0.05% to about 4% of an antibacterial agent, from about 0.0005% to about 3% of an anti-caries agent, from about 0.1% to about 5% of a flavoring agent, and from about 0.1% to about 5% of a sweetening agent.

Gels include dentrifice gels (see description above), non-abrasive gels and subgingival gels. Non-abrasive gels and subgingival gels generally include a thickening agent, a humectant, a flavoring agent, a sweetening agent, a coloring agent, and water. Such gels may also include one or more anti-caries agents and/or anti-calculus agents. Typically, such a gel will contain from about 0.1% to about 20% of a thickening agent, from about 10% to about 55% of a humectant, from about 0.04% to about 2% of a flavoring agent, from about 0.1% to about 3% of a sweetening agent, from about 0.01% to about 0.5% of a coloring agent, and the balance water. Such gels may also contain from about 0.05% to about 0.3% of an anti-caries agent and from about 0.1% to about 13% of an anti-calculus agent.

Creams generally include a thickening agent, a humectant and a surfactant, and may include a flavoring agent, a sweetening agent, a coloring agent. Typically, a cream will contain from about 0.1% to about 30% of a thickening agent, from about 0% to about 80% of a humectant, from about 0.1 % to about 5% of a surfactant, from about 0.04% to about 2% of a flavoring agent, from about 0.1% to about 3% of a sweetening agent, from about 0.01% to about 0.5% of a coloring agent, and from about 2% to about 45% of water.

Ointments suitable for oral use are described in, e.g., U.S. Pat. Nos. 4,847,283, 5,855,872 and 5,858,408, the complete disclosures of which are incorporated herein by reference. Ointments generally include one or more of the following: fats, oils, waxes, parafins, silicones, plastibase, alcohols, water, humectants, surfactants, thickening agents, talc, bentonites, zinc oxide, aluminum compounds, preservatives, antiviral compounds, and other ingredients. For instance, the ointment may comprise from about 80% to about 90% petrolatum and from about 10% to about 20% ethanol or propylene glycol. As another example, the ointment may comprise about 10 % petrolatum, about 9% lanolin, about 8% talc, about 32% cod liver oil, and about 40% zinc oxide. As a third example, the ointment may comprise from about 30% to about 45% water, from about 10% to about 30% oil (e.g.,petrolatum or mineral oil), from about 0.1% to about 10% emulsifier (e.g., wax NF), from about 2% to about 20% humectant (e.g., propylene glycol), from about 0.05% to about 2% preservatives (e.g., methyl paraben and propyl paraben), and from about 10% to about 40% sterol alcohol.

Mouthwashes, rinses, gargles and sprays generally include water, ethanol, and/or a humectant, and preferably also include a surfactant, a flavoring agent, a sweetening agent, and a coloring agent, and may include a thickening agent and one or more anti-caries agents and/or anti-calculus agents. A typical composition contains from about 0% to about 80% of a humectant, from about 0.01% to about 7% of a surfactant, from about 0.03% to about 2% of a flavoring agent, from about 0.005% to about 3% of a sweetening agent, from about 0.001% to about 0.5% of a coloring agent, with the balance being water. Another typical composition contains from about 5% to about 60%, preferably from about 5% to about 20%, ethanol, from about 0% to about 30%, preferably from about 5% to about 20%, of a humectant, from about 0% to about 2% emulsifying agents, from about 0% to about 0.5% of a sweetening agent, from about 0% to about 0.3% of a flavoring agent, and the balance water. A further typical composition contains from about 45% to about 95% water, from about 0% to about 25%, ethanol, from about 0% to about 50% of a humectant, from about 0.1% to about 7% of a surfactant, from about 0.1% to about 3% of a sweetening agent, from about 0.4% to about 2% of a flavoring agent, and from about 0.001% to about 0.5% of a coloring agent. These compositions may also comprise from about 0.05% to about 0.3% of an anti-caries agent, and from about 0.1% to about 3% of an anti-calculus agent

Solutions generally include water, a preservative, a flavoring agent, and a sweetening agent, and may include a thickening agent and/or a surfactant. Typically, solutions contain from about 85% to about 99% water, from about 0.01% to about 0.5% of a preservative, from about 0% to about 5% of a thickening agent, from about 0.04% to about 2% of a flavoring agent, from about 0.1% to about 3% of a sweetening agent, and from about 0% to about 5% of a surfactant. Another simple solution that can be used is a saline solution, a buffer solution, or buffered saline, optionally containing a preservative, a thickening agent and/or a surfactant.

Lozenges and mints generally include a base, a flavoring agent and a sweetening agent. The base may be a candy base (hard sugar candy), glycerinated gelatin or a combination of sugar with sufficient mucilage to give it form. See U.S. Pat. No.6,350,438 and Remington, The Science And Practice Of pharmacy, 19^{th} edition (1995). Lozenge compositions also typically include one or more fillers (e.g., a compressible sugar) and lubricants.

Chewing gums, chewable tablets and chewable lozenges are described in U.S. Pat. Nos. 6,471,991, 6,296,868, 6,146,661, 6,060,078, 5,869,095, 5,709,873, 5,476,647, and 5,312,626, PCT applications WO 84/04453 and WO 99/02137, and Lieberman et al., Pharmaceutical Dosage Forms, 2^{nd} ed. (1990), the complete disclosures of which are incorporated here in by reference.

As one example, a compressed chewable tablet comprises a water-disintegratable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including the metal binding compound(s) of the invention, are dry blended and compressed into a tablet.

As another example, a chewable tablet may comprise a core surrounded by an outer layer wrapping the core. The core may comprise a metal binding compound or compounds of the invention and, optionally, other active ingredients in a jelly base or a chewable base. The outer layer may be a chewable base. The jelly base may comprise pectin, sorbitol, maltitol, isomalt, liquid glucose, sugar, citric acid and/or a flavoring agent. The chewable base of the core or outer layer may be a gum, soft candy, nougat, caramel or hard candy. The tablets are formed by extrusion of the core and outer layer to form a rope, followed by cutting the rope into tablets.

Chewing gum compositions generally include a gum base, a flavoring agent and a sweetening agent. Suitable gum bases include jelutong, rubber, latex, chicle, and vinylite resins, desirably with conventional plasticizers or softeners. Plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. The metal-binding compound(s) of the invention and other active ingredients may be incorporated into a gum base by, e.g., stirring them into a warm gum base or coating them onto the outer surface of the gum base.

Films and sheets, and gels which form solids in the mouth, made of lactide/glycolide copolymers are described in U.S. Pat. Nos.5,198,220, 5,242,910 and 6,350,438. Another polymer film suitable for use in the mouth is described in PCT application WO 95/32707. Patches that adhere to hard dental surfaces, such as teeth and dentures, and which degrade in the mouth, are described in U.S. Pat. No. 6,197,331. All of these materials slowly release active agents contained in them into the mouth. Other compositions (including pastes, gels, ointments, liquids and films) providing for slow release of active agents are also known. See, e.g., U.S. Pat. Nos. 5,032,384, 5,298,237, 5,466,437, 5,709,873, and 6,270,781.

Tooth whitening compositions will comprise a tooth whitening agent. Tooth whitening agents include peroxides, percarbonates and perborates of the alkali and alkaline earth metals or complex compounds containing hydrogen peroxide. Tooth whitening agents also include peroxide salts of the alkali or alkaline earth metals. The most commonly used tooth whitening agent is carbamide peroxide. Other commonly used tooth whitening agents are hydrogen peroxide, peroxyacetic acid and sodium perborate. These tooth whitening agents liberate active oxygen and hydrogen peroxide. Tooth whitening agents can be present in tooth whitening compositions at a concentration of from about 0.1% to about 90%; typically, the concentration of carbamide peroxide in tooth whitening compositions is from about 10% to about 25%.

Many tooth whitening compositions are known in the art, including aqueous solutions, gels, pastes, liquids, films, strips, one-part systems, two-part systems, compositions that require activation of the tooth whitening agent (e.g., by inclusion of a radiant-energy or heat-energy absorbing substance, such as substantially conjugated hydrocarbons, which activates. the bleaching agent when irradiated), etc. See, e.g., U.S. Pat. Nos. 5,302,375, 5,785,887, 5,858,332, 5,891,453, 5,922,307, 6,322,773, 6,419,906, and PCT applications WO 99/37236, WO 01/89463 and WO 02/07695, the complete disclosures of which are incorporated herein by reference. Also, many other oral care compositions (e.g., toothpastes) and devices (e.g., dental flosses) comprise a tooth whitening agent.

The use of tooth whitening compositions, or of one of the many oral care compositions and devices which comprise a tooth whitening agent, results in the production of ROS and can cause inflammation of the tissues of the mouth. Incorporation of a metal-binding compound or compounds of the invention in tooth whitening compositions or other oral care compositions and devices comprising a tooth whitening agent will reduce or prevent the inflammation and/or the production of ROS. The inclusion of a metal-binding compound or compounds of the invention in such compositions may also result in more effective whitening, since hydrogen peroxide, which is responsible for the whitening of teeth by the hydrogen peroxide-type whitening agents, will not be converted into hydroxyl radicals (see Examples 8 and 9) and will, therefore, remain active longer. Alternatively, an oral care composition or device comprising a metal-binding compound or compounds of the invention can be used before or after the tooth whitening composition or oral care composition or device comprising a tooth whitening agent to reduce or prevent the inflammation and/or the production of ROS.

For instance, teeth are commonly whitened by applying a tooth whitening composition to the teeth by means of a dental tray or trough. A metal-binding compound or compounds of the invention could be incorporated into the tooth whitening composition that is used in the tray or trough. Alternatively, a separate composition comprising a metal binding compound or compounds of the invention could be applied to the teeth in a cleaned or different tray or trough after the application of the tooth whitening composition is completed. In a further alternative, a wash or rinse comprising a metal binding compound or compounds of the invention could be used to rinse the mouth before and/or after the application of the tooth whitening composition.

A recently developed product for applying a tooth whitening composition to the teeth is a flexible strip. See, e.g., U.S. Pat. Nos.5,891,453 and 6,419,906. A metal-binding compound or compounds of the invention could be incorporated into such strips. For instance, the metal-binding compound(s) could be incorporated into the tooth whitening composition, which is then applied to the strips, or a solution, gel or other composition comprising the metal-binding compound(s) could be separately applied to the strips, either during their manufacture or just prior to use by the patient. In yet another alternative, strips comprising a tooth whitening composition and strips comprising the metal binding compound(s) could both be supplied to the patient and would be used sequentially.

The oral care compositions of the invention may comprise a single phase or a plurality of phases. A plurality of phases will be used, e.g., where some of the ingredients are incompatible, some of the ingredients are unstable, or the ingredients are best combined at the time of use. Thus, one of the phases will include some of the ingredients, and the remainder of the ingredients will be contained in one or more additional phases. The plurality of phases may be a plurality of separate compositions, in which case the plurality of phases will be provided in a plurality of separate containers or in a plurality of compartments in a single container, and the plurality of phases will be combined at the time of use. As an alternative the plurality of phases may be formed by encapsulating some of the ingredients, in which case the plurality of phases may all be contained in a single container. Multi-phase oral care compositions are described in, e.g., U.S. Pat. Nos. 5,302,.375, 5,906,811, 5,976,507, 6,228,347 and 6,350,438 and PCT application number WO 99/37236.

The invention also provides oral care devices comprising a metal-binding compound or compound(s). Oral care devices of the invention include devices intended for use by consumers and patients and devices intended for use by dental professionals (e.g., dental hygienists, dentists and oral surgeons).

The oral care devices of the invention include surgical materials (such as sutures and sponges), flosses, tapes, chips, strips, fibers, a toothpick or rubber tip, dental implants and dental appliances (such as trays and troughs that fit over and cover the teeth and, optionally, the periodontal tissue) having a metal-binding compound or compounds of the invention adhered to, absorbed into, bound to, attached to, entrapped in, coated onto, or otherwise incorporated into, them. See, e.g., U.S. Pat. Nos. 5,709,873, 5,863,202, 5,891,453, 5,967,155, 5,972,366, 5,980,249, 6,026,829, 6,080,481, 6,102,050, 6,350,438, 6,419,906, PCT application WO 02/13775, and EP application 752833, which describe such oral care devices and methods of incorporating compounds into them (the complete disclosures of all of these patents and applications are incorporated herein by reference). For instance, a metal-binding compound or compounds of the invention can be incorporated into a binder (e.g., a wax or polymer) and coated onto dental floss, dental floss can be soaked in a bath of a liquid containing a metal-binding compound or compounds of the invention to impregnate or coat the floss with the compound(s), a metal-binding compound or compounds of the invention in solid (e.g., freeze-dried) form can be incorporated into a polymer film suitable for application to the teeth, a metal-binding compound or compounds of the invention in a solution or gel can be applied to a flexible strip suitable for application to teeth, or a suture or other surgical material can be soaked in a solution containing a metal-binding compound or compounds of the invention followed by removal of the solvent so that the compound(s) become associated with (bound to, entrapped in, coated onto, etc.) the suture or surgical material. See, e.g., U.S. Pat. Nos. 5,891,453, 5,967,155, 5,972,366, 6,026,829, 6,080,481, 6,102,050, and 6,419,906.

Also included within the scope of the invention are oral care products for animals, such as foods, chews, and toys. Suitable products are described in U.S. Pat. No. 6,350,438.

A metal-binding compound or compounds of the invention can be used to treat a tissue of an animal's mouth. “Mouth” is used herein to mean the cavity bounded externally by the lips and internally by the pharynx that encloses the tongue, gums and teeth. Thus, the tissues of the mouth include the lips, tongue, gums, buccal tissue, palate and teeth. A single tissue, a plurality of tissues, a portion of one or more tissues, all or substantially all of the tissues of the mouth, or combinations of the foregoing, may be treated according to the invention. “Treat” and variations thereof are used herein to mean to cure, ameliorate, alleviate, inhibit, prevent, reduce the likelihood of, or reduce the severity of, a disease or condition, or of at least some of the symptoms or effects thereof.

To treat a tissue of the mouth, the tissue is contacted with a metal-binding compound or compounds of the invention. For instance, the tissue may be contacted with an oral care composition comprising the metal-binding compound(s). Methods of contacting tissues of the mouth with oral care compositions are well known in the art. Suitable methods include rinsing the tissue with a solution (e.g., a mouthwash, rinse, spray, liquid dentrifice, or other solution), brushing the teeth with a dentrifice (e.g., a toothpaste, tooth gel, or powder), applying a non-abrasive solution, gel, paste, cream or ointment directly to the tissue (with or without the use of an applicator), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip (such as those described in U.S. Pat. Nos.5,891,453 and 6,419,906), a finger, or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. In addition, to treat a tissue of the mouth, the tissue may be contacted with an oral care device comprising the metal-binding compound(s). Methods of contacting tissues of the mouth with oral care devices are well known in the art. For instance, sutures can be used to close a surgical wound or a wound resulting from a tooth extraction, dental floss can be used to floss the teeth, etc.

The treatment of the tissue can be prophylactic treatment. For instance, the tissue may be treated as part of a prophylactic oral care regimen. The metal-binding compound(s) of the invention can be incorporated into an oral care composition or device, such as a toothpaste, a tooth gel, a mouthwash or rinse, or a dental floss, that is employed in such a regimen and will be used preferably at least once per day, more preferably two or three times per day. In another alternative, the metal-binding compound(s) of the invention may be contained in a separate oral care composition or device which will be used separately from other compositions and devices employed in the prophylactic oral care regimen. For instance, the metal-binding compound(s) of the invention can be incorporated into a mouthwash or rinse, a gum, a lozenge or a chewable tablet, which would preferably be used at least once per day, more preferably at least two or three times per day. It may be particularly beneficial for those patients who utilize tobacco products to use the metal-binding compound(s) of the invention as part of a prophylactic oral care regimen to attempt to ameliorate the damage done to tissues of the mouth by such products.

It is Inown to include metal salts, particularly copper salts, in toothpastes and other oral care compositions, generally as antibacterial, anti-plaque, anti-caries, and anti-gingivitis agents. See, e.g., U.S. Pat. Nos. 5,286,479, 5,298,237, and 6,355,706, EP application 658,565, PCT application WO 92/08441, Japanese application 41 59211, Waerhaug et al., J. Clin. Periodontol., 11:176-180 (1984). The use of oral care compositions containing copper salts could be harmful to the tissues of the mouth, since free copper ions catalyze the formation of ROS. Thus, the use of an oral care composition of the present invention at an appropriate time after the use of the copper-containing compositions (i.e., allowing sufficient time for the copper salts to exert their activity) could be very beneficial in reducing the damage done by ROS generated by copper ions present in the mouth as a result of use of these products. For instance, the metal-binding compound(s) could conveniently be supplied in a gum, lozenge or chewable table which would be chewed or sucked after use of the copper-containing compositions.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising the metal-binding compound(s), the wound(s) caused by the surgery or tooth extraction could be closed with sutures having the metal-binding compound(s) incorporated into them, and/or the mouth could be rinsed immediately after the surgery or tooth extraction, and/or at intervals thereafter, with a solution comprising the metal-binding compound(s).

Tissues can also be treated prophylactically in connection with radiation, such as dental x-rays. Finally, as described above, tissues may be treated prophylactically in connection with the whitening of the teeth of an animal.

A metal-binding compound or compounds of the invention can be used to treat a disease or condition of a tissue of an animal's mouth. Diseases and conditions treatable according to the invention include infections (bacterial infections, viral infections and fungal infections), inflammation due to any cause, and any disease or condition involving or caused by metal ions and/or ROS. Specific diseases and conditions treatable according to the invention include diseases of the periodontal tissue, such as gingivitis and periodontitis, ulcers, cold sores, canker sores, other viral infections, bacterial infections and yeast and fungal infections.

It is understood by those skilled in the art that the dosage amount of the metal-binding compound(s) of the invention needed to treat a tissue of an animal's mouth will vary with the particular metal-binding compound employed, whether the treatment is prophylactic or for the treatment of a disease or condition, the identity of the disease or condition to be treated, the severity of the disease or condition, the type of oral care composition used, the duration of the treatment, the identify of any other drugs being administered to the animal, the age, size and species of the animal, and like factors known in the medical and veterinary arts. In general, a suitable daily dose of a compound of the present invention will be that amount of the compound which is the lowest dose effective to produce a therapeutic effect. As noted above, it is expected that usage of oral care compositions comprising from about 0.001% to about 20% of a metal binding compound or compounds of the invention one or more times per day will provide effective daily dosages. However, the actual daily dosage to be employed, the number of treatments per day, and the length of treatment will be determined by an attending physician or veterinarian within the scope of sound medical judgment.

The invention also provides a kit comprising an oral care product according to the invention. In the case where the oral care product is an oral care composition, the kit may also include an applicator for applying the oral care composition to a tissue of an animal's mouth, such as a swab, a stick, a plastic paddle, a dropper, a syringe, a strip (such as that described in U.S. Pat. Nos.5,891,453 and 6,419,906) or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. The kit could also include a cup, vial or other device for dispensing and/or measuring the amount of the oral care composition of the invention needed for the intended use. Of course, the kits could include both an oral care composition and an oral care device according to the invention. In addition to an oral care composition and/or device of the invention, the kits could also comprise another type of oral care composition or device, such as a tooth whitening composition, strips comprising a tooth whitening agent, applicators for applying oral care compositions, etc. Kits according to the invention will also include instructions for using the kit and/or the oral care product of the invention and may include any other desired items.

It is to be noted that “a” or “an” entity refers to one or more of that entity. For example, “a cell” refers to one or more cells.

### EXAMPLES

### EXAMPLE 1

Synthesis of Tetrapeptide Asp Ala His Lys [SEQ ID NO:1]

This example describes the synthesis of the tetrapeptide Asp Ala His Lys [SEQ ID NO: 1] composed of all L-amino acids using standard solid-phase synthesis techniques. First, 9-fluorenylmethyloxycarbonyl (Fmoc)-protected Asp (v COO— ester; Tolsulfonyl) on Wang resin (0.6 mmole; Nova Biochem) was suspended in a solution of piperidine/dimethylformamide (DMF) (40% v/v; 3 ml) for 30 min with occasional agitation. At the end of this period, the solvent was drained, and the resin was washed sequentially with DMF and dichloromethane (DCM; 5×3 ml). A ninhydrin test was used to monitor the reaction. The resin was swollen with DMF (˜1 ml). The C-protected t-benzyloxycarbonyl (Boc) ester of alanine in DMF was added, followed by a mixture of diisopropylamine (8 equivalent) and 2-(1H-benzotriazole-1-yl)-1,2,3,3-tetramethyluroniumtetrafluoroborate (TBTU-) (4 equivalents). The resin was shaken for about 24 hours, and the reaction was monitored by the ninhydrin test. At the end of this period, DMF was drained, and the resin was washed with DMF and DCM. The solution was drained, and the beads were washed with DCM (3×2 ml). The protecting group of the dipeptide-resin was removed, and the beads were suspended in DMF. Amino protected (benzyloxy) derivative of histidine (4 mmole) was added, followed by mixture of diisopropylamine (8 equivalent) and TBTU- (4 equivalent). The resin was shaken for about 24 hours, and the reaction monitored by ninhydrin test. At the end of this period, DMF was drained, and the resin was washed with DMF and DCM. The tripeptide-resin was briefly dried in a gentle stream of nitrogen and suspended in nitrogen-saturated DMF. Protected lysine was added, followed by a mixture of diisopropylamine (8 equivalent) and TBTU- (4 equivalent). The resin was shaken for about 24 hours, and the reaction monitored by the ninhydrin test. At the end of this period, DMF was drained and the resin was washed with DMF and DCM. The Boc protecting group was carefully removed to give the tetrapeptide bound to the resin, with a typical loading of 5 mmole/g. The resin bound tetrapeptide (0.25 gm; 5 mmolar) was treated with trifluoroacetic acid (TFA) and was shaken for 24 hours. At the end of this period, the ninhydrin test gave a blue color, indicating the release of the tetrapeptide from the resin. In some circumstances, addition of 5%(V/V) of DMF to TFA accelerated the rate of release of the peptide from the resin. Removal of TFA at reduced pressure gave the tetrapeptide (all D) as TFA salt and was dried under vacuum at 5° C. for 24 hours. The residue was a white powder and was characterized by spectrometric methods.

A number of enantiomers of the tetrapeptide can be prepared in this manner. For example, use of D-amino acids in the peptide synthesis forms the tetrapeptide containing all D-amino acids. Also, combinations of L-amino acids and D-amino acids can be used.

### EXAMPLE 2

Preparation of Cyclohexanediamine Derivative of Asp Ala His Lys [SEQ ID NO:1]

Trans-diaminocyclohexane was prepared by resolving cis/trans 1,2-diaminocyclohexane (Aldrich-Sigma) as the tartaric acid salt. The R-trans isomer melts at 75° C. and the S-trans isomer melts between 43-45° C. (Ph.D. Thesis, P. D. Newman, University College, Cardiff, U.K., 1994). The trans-diaminocyclohexane (10 gm) was then suspended in anhydrous toluene (30 mL) and cooled to 5° C. in an ice bath, and bromoacetic acid (8 gm) in toluene (25 mL) was added dropwise. At the end of the addition, the reaction temperature was raised to 30° C. and kept at that temperature for a further 5 hours. Toluene was evaporated, and the R-trans 1,2-diaminocyclohexane diacetic acid was crystallized from hexane/toluene to give a white solid (yield 70%). The product was characterized by spectroscopic methods.

The resin-bound tetrapeptide prepared in Example 1 (20 mg) was suspended in DMF (5 mL) and was treated with the R-trans 1,2-diaminocyclohexanediacetic acid (20 mg) followed by addition of a mixture of diisopropylamine (8 equivalent) and TBTU-(4 equivalent). The resin was shaken for about 24 h on a roller. Then, the resin was washed with DMF followed by DCM (5×3 mL) and partially dried. Hydrolysis of the resin linkage was effected by treating the resin-bound reaction product with TFA (5 mL; 5 hr). The resin was separated and washed with DCM. The washings were combined with TFA and concentrated under vacuum. The residue (cyclohexanediamine tetrapeptide; formula given in FIG. 3D where R₅ is H) was characterized by spectrometric analysis.

### EXAMPLE 3

Preparation of Tetrapeptide Tetracetic Acid

The resin-bound tetrapeptide prepared in Example 1 (20 mg) was suspended in DMF (5 mL) and treated with excess (10-fold) chloroacetic acid. The resin was shaken at room temperature for 48 hours, followed by heating to 60° C. for a further hour. DMF was removed by filtration, and the resin was washed with DMF followed by DCM (5×3 mL). Partially dried resin was used without further treatment in the next stage. Hydrolysis of the resin linkage was effected by treating the resin-bound reaction product with TFA (5 mL; 5 hr). The resin was separated and washed with DCM. The washings were combined with TFA and concentrated under vacuum (yield 30%). The product (formula given in FIG. 4) was characterized by spectrometric methods.

### EXAMPLE 4

Preparation of Mesoporphyrin IX Tetrapeptide

The resin-bound tetrapeptide prepared in Example 1 (20 mg) was suspended in DMF (5 mL) and treated with mesoporphyrin IX dicarboxylic acid (10 μmole; formula given in FIG. 6A), followed by addition of a mixture of diisopropylamine (8 equivalent) and TBTU- (4 equivalent). The resin was shaken for about 24 hours on a roller kept in a dark chamber. The resin was washed with DMF followed by DCM (5×3 mL) and partially dried. Hydrolysis of the resin linkage was effected by treating the resin-bound reaction product with TFA (5 mL; 5 hr). The resin was separated and washed with DCM/TFA mixture (1:1.5 mL). The washings were combined and concentrated under vacuum. The porphyrin tetrapeptide (formula given in FIG. 6C) was purified by semi-preparative HPLC (yield 60%). The structure was confirmed by spectrometric methods.

This procedure can be used to synthesize other porphyrin-peptides, such as mesoporphyrin I and related molecules.

### EXAMPLE 5

Preparation of Tetrabispiridylethyl Tetrapeptide

The resin-bound tetrapeptide prepared in Example 1 (20 mg) was suspended in DMF (5 mL) and treated with bromoethylpyridine (20 μmole). This was followed by the addition of pyridine (0.5 mL). The resin was shaken for about 48 hours on a roller. The resin was washed with DMF, followed by DCM (5×3 mL) to remove all of the unreacted monomers, and then dried under vacuum for 30 minutes. Hydrolysis of the resin linkage was effected by treating the resin-bound reaction product with TFA (5 mL; 5 hr). The resin was separated and washed with DCM/TFA mixture (1:1.5 mL). The washings were combined and concentrated under vacuum. The pyridylethyl tetrapeptide derivative (formula given in FIG. 5) was purified by semi-preparative HPLC (yield 50%). The structure was confirmed by spectrometric methods.

This procedure can be applied to other heterocycles, such as phenanthroline and related molecules.

### EXAMPLE 6

Preparation of Aryl Derivative of Asp Ala His Lys [SEQ ID NO:1]

A derivative having the formula shown in FIG. 1B, wherein R₁ is phenyl, was prepared. Diethylacetamidomalonate (10 gm) in anhydrous ethanol (100 mL) was added to a slurry of sodium ethoxide in ethanol (5 gm; 50 mL) and heated to reflux for 30 min. The product was cooled (10° C.) and reacted with ethyl α-bromophenyl acetate (5 gm). The reaction was allowed to proceed to completion (24 h), and excess sodium ethoxide was neutralized with dilute acid. The triester was extracted into ethylacetate and, upon removal of solvent, gave a viscous liquid. The crude product was hydrolyzed with hydrochloric acid (100 mL) and decarboxylated to give phenyl substituted aspartic acid (10 gm). The N-benzoyloxy t-butyl derivative was prepared using a standard reaction sequence. To the resin-bound tripeptide (Lys His Ala) prepared as described in Example 1 (20 mg) in DMF was added the N-benzoyloxy-t-butyl aspartic acid derivative, followed by a mixture of diisopropylamine (8 equivalent) and TBTU-(4 equivalent). The resin was shaken for about 24 h, and the reaction monitored by the ninhydrin test. At the end of this period, DMF was drained, and the resin was washed with DMF and DCM. The solution was drained, and the beads washed with DCM (3×2 ml). The tetrapeptide derivative was isolated by careful hydrolysis. Stereoisomers of the tetrapeptide were separated by preparative-scale HPLC.

### EXAMPLE 7

Inhibition Of The Generation Of ROS By The Tetrapeptide Asp Ala His Lys [SEQ ID NO: 1]

A tetrapeptide having the sequence L-Asp L-Ala L-His L-Lys [SEQ ID NO:1] (the L-tetrapeptide) was obtained from one or more companies that provide custom synthesis of peptides, including Ansynth Services, QCB, Genosys and Bowman Research. The peptide was prepared by standard solid phase synthesis methods (see also Example 1).

The ability of the L-tetrapeptide to inhibit the generation of ROS was tested as described in Gutteridge and Wilkins, Biochim. Biophys. Acta, 759, 38-41 (1983) and Cheeseman et al., Biochem. J., 252, 649-653 (1988). Briefly, Cu(II) and H₂O₂ were mixed causing the generation of hydroxyl radicals in a Fenton-type reaction. The hydroxyl radicals attack the sugar 2-deoxy-D-ribose (the sugar residue of DNA) to produce fragments. Heating the fragments at low pH produces malonaldehyde that, upon the addition of 2-thiobarbituric acid, yields a pink chromogen which can be measured spectrophotometrically at 532 nm. Thus, the absorbance at 532 nm is a measure of the damage to 2-deoxy-D-ribose.

The assay was performed with and without the L-tetrapeptide. The results are summarized in Table 1. As can be seen from Table 1, when the L-tetrapeptide was present at Cu(II):tetrapeptide ratios of 1:1.2 and 1:2, the degradation of 2-deoxy-D-ribose was inhibited by 38% and 73%, respectively. Clearly, the L-tetrapeptide inhibited the degradation of 2-deoxy-D-ribose by hydroxyl radicals.

TABLE 1

| | CuCl₂ | H₂O₂ | Tetra-peptide | OD at 532 | Percent |
|---|---|---|---|---|---|
| | (mM) | (mM) | (mM) | nm | Inhibition |
| Control | 0.1 | 2 0 | 0.0 | 0.124 | |
| Tetrapeptide | 0.1 | 2.0 | 0.12 | 0.077 | 38 |
| Control | 0 1 | 2 0 | 0.0 | 0.175 | |
| Tetrapeptide | 0.1 | 2 0 | 0.2 | 0.048 | 73 |

A similar assay was also performed using a tetrapeptide having the sequence Asp Ala His Lys composed of all D-amino acids (D-tetrapeptide). The D-tetrapeptide was obtained from one or more companies that provide custom synthesis of peptides, including Ansynth Services and QCB. The peptide was prepared by standard solid phase synthesis methods (see Example 1)

The ability of the D-tetrapeptide to inhibit the generation of ROS was tested as described by Zhao and Jung, Free Radic Res, 23(3), 229-43 (1995). Briefly, Cu(II) and ascorbic acid were mixed causing the generation of hydroxyl radicals in a Fenton-type reaction. The advantage of using ascorbic acid instead of hydrogen peroxide is that ascorbic acid does not interfere with other assays (e.g., LDH assay) which is not the case with peroxide. The hydroxyl radicals attack the sugar 2-deoxy-D-ribose to produce fragments. Heating the fragments at low pH produces malonaldehyde that, upon the addition of 2-thiobarbituric acid, yields a pink chromogen which can be measured spectrophotometrically at 532 nm. Thus, the absorbance at 532 nm is a measure of the damage to 2-deoxy-D-ribose.

Establishing optimal Cu(II) and ascorbic acid concentrations was the first step in developing this protocol. First, a constant Cu(II) concentration of 10 μM was used based on this level being the physiological concentration found in the body (bound and unbound Cu(II)). The ascorbic acid concentrations were varied in order to establish a linear range. The ascorbic acid concentration chosen was 500 μM since it gave the most absorbance at 532 nm and still fell in the linear range. Interestingly, at ascorbic acid concentrations greater than 500 μM, there was a steady decrease in hydroxyl radicals presumably due to ascorbic acid's dual effect as a hydroxyl radical generator at low concentrations and an antioxidant at high concentrations.

Using the aforementioned concentrations for Cu(II) and ascorbic acid, a titration curve was established for the D-tetrapeptide. Briefly, the D-tetrapeptide was pre-incubated with Cu(II) for 15 minutes at room temperature prior to adding ascorbic acid. This was done to permit the D-tetrapeptide to bind with the Cu(II) and therefore inhibit ROS generation. As can be seen from the table, when the Cu(II):D-tetrapeptide ratio was between 4:1 to 4:7, there was little to no inhibition of hydroxyl radical generation. When the ratio was 1:2 or higher, there was total inhibition of hydroxyl radical production.

TABLE 2

| Cu(II):D- | Cu(II) | Ascorbic | D-Tetrapeptide | | % |
|---|---|---|---|---|---|
| Tetrapeptide | (μM) | Acid (μM) | (μM) | A532 | Inhibition |
| 1:0 | 10 | 500 | 0 | 0.767 | |
| 4:1 | 10 | 500 | 2.5 | 0.751 | |
| 2:1 | 10 | 500 | 5 | 0.743 | |
| 1:1 | 10 | 500 | 10 | 0.751 | |
| 4:5 | 10 | 500 | 12.5 | 0.789 | |
| 2:3 | 10 | 500 | 15 | 0 774 | |
| 4:7 | 10 | 500 | 17.5 | 0.737 | |
| 1:2 | 10 | 500 | 20 | 0.029 | 96 2 |
| 1:4 | 10 | 500 | 40 | 0.016 | 97.9 |

### EXAMPLE 8

Inhibition Of The Generation Of ROS

The ability of the tetrapeptide L-Asp L-Ala L-His L-Lys [SEQ ID NO: 1] and other peptides and compounds to inhibit the production of ROS was tested. The other peptides tested were: L-Asp L-Ala L-His L-Lys L-Ser L-Glu L-Val L-Ala L-His L-Arg L-Phe L-Lys [SEQ ID NO:3]; L-Ala L-His L-Lys L-Ser L-Glu L-Val L-Ala L-His L-Arg L-Phe L-Lys [SEQ ID NO:4]; L-His L-Lys L-Ser L-Glu L-Val L-Ala L-His L-Arg L-Phe L-Lys [SEQ ID NO:5]; and Acetylated-L-Asp L-Ala L-His L-Lys L-Ser L-Glu L-Val L-Ala L-His L-Arg L-Phe L-Lys [SEQ ID NO:6]. The peptides were obtained from one or more companies that provide custom synthesis of peptides, including Ansynth Services, QCB, Genosys and Bowman Research. The other compounds tested were histidine (Sigma Chemical Co.), catalase (Sigma Chemical Co.), and superoxide dismutase (Sigma Chemical Co.).

1. Inhibition Of Hydroxyl Radical Production

The hydroxyl radical is probably the most reactive oxygen-derived species. The hydroxyl free radical is very energetic, short-lived and toxic.

Some researchers suggest that the toxicity of hydrogen peroxide and superoxide radical may be due to their conversion to the hydroxyl free radical. The superoxide radical can be directly converted to the hydroxyl radical via the Haber-Weiss reaction. Alternatively, it can be converted to hydrogen peroxide which, in turn, is converted into the hydroxyl radical via the Fenton reaction. Both pathways require a transition metal, such as copper (Acworth and Bailey, The Handbook Of Oxidative Metabolism (ESA, Inc. 1997)).

It is also known that copper, in the presence of ascorbate, produces hydroxyl radicals. The following reaction scheme has been suggested:

Ascorbate+2Cu²⁺→2Cu⁺ dehydroascorbate+2H⁺ (Eq. 1)

Cu⁺+O₂→O₂^{.-}+Cu²⁺ (Eq. 2)

Cu⁺+O₂^{.-}+2H⁺→Cu²⁺+H₂O₂ (Eq. 3)

Cu⁺+H₂O₂→OH⁻+OH³¹ +Cu²⁺ (Eq. 4)

Biaglow et al., Free Radic. Biol. Med., 22(7):1129-1138 (1997).

The ability of the compounds listed above to inhibit the generation of hydroxyl radicals was tested as described in Gutteridge and Wilkins, Biochim. Biophys. Acta, 759:38-41 (1983). Briefly, Cu(II) and ascorbic acid were mixed causing the generation of hydroxyl radicals. Then, deoxyribose was added, and the hydroxyl radicals, if present, attacked the deoxyribose to produce fragments. Heating the fragments at low pH produced malonaldehyde that, upon the addition of 2-thiobarbituric acid (TBA), yielded a pink chromogen which was measured spectrophotometrically at 532 nm. Thus, absorbance at 532 nm is a measure of the damage to deoxyribose and, therefore, of hydroxyl radical formation.

To perform the assay, CuCl₂ in buffer (20 mM KH₂PO₄ buffer, pH 7.4) and either one of the test compounds in buffer or buffer alone were added to test tubes (final concentration of CuCl₂ was 10 μM). The test tubes were incubated for 15 minutes at room temperature. Then, 0.5 mM ascorbic acid in buffer and 1.9 mM 2-deoxy-D-ribose in buffer were added to each test tube, and the test tubes were incubated for 1 hour at 37° C. Finally, 1 ml of 1% (w/v) TBA in 50 mM NaOH and 1 ml of concentrated acetic acid were added to each test tube, and the test tubes were incubated in boiling water for 15 minutes. After the test tubes had cooled for 15 minutes, the absorbance at 532 nm was read.

It was found that the tetrapeptide L-Asp L-Ala L-His L-Lys [SEQ ID NO:1] caused complete inhibition of the formation of hydroxyl radicals in this assay at tetrapeptide/copper ratios of 2:1 or higher. Tetrapeptide/copper ratios less than 2:1 were ineffective.

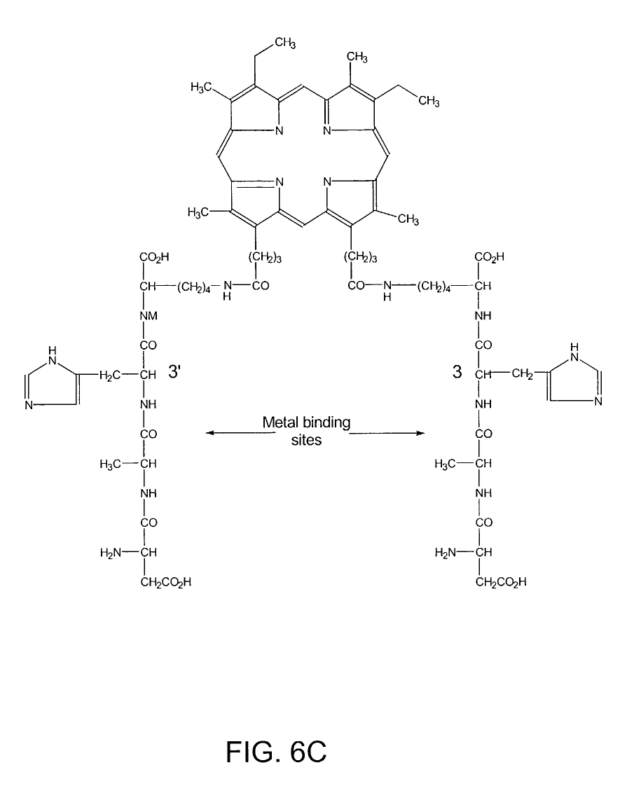

The results of a time course are presented in FIG. 8A. As can be seen in FIG. 8A, copper and ascorbate (no added peptide) produced TBA-reactive substances quickly and reached a maximum in 30 minutes. The tetrapeptide at a tetrapeptide/copper ratio of 2:1 prevented all formation of TBA-reactive substances. Interestingly, the tetrapeptide at a tetrapeptide/copper ratio of 1:1 slowed the production of TBA-reactive substances. These data suggest that the tetrapeptide at a 1:1 tetrapeptide/copper ratio is able to offer some protection from hydroxyl radicals by binding copper which results in site-directed hydroxyl attack on the.tetrapeptide. Once enough of the tetrapeptide is destroyed, then copper is released, which allows it to produce hydroxyl radicals that attack the dexoyribose.

When the tetrapeptide at a tetrapeptide/copper ratio of 2:1 was incubated for longer periods of time, its ability to prevent the formation of TBA-reactive substances slowly eroded. See FIG. 8B. As can be seen from FIG. 8B, the production of TBA-reactive substances was inhibited by 95% during the first 4 hours of incubation. By 24 hours, the level of inhibition had dropped to 50% and, by 48 hours, the level of inhibition had dropped to 20%. These data suggest that TBA-reactive substances are still being produced even in the presence of the tetrapeptide. They also suggest that the tetrapeptide is being degraded during the time course of the experiment. This degradation is more than likely due to the formation of free radicals in close proximity to the tetrapeptide/copper complex which attack and degrade the tetrapeptide, with release of the copper. Since free radicals, such as the hydroxyl radical, are very reactive, they will attack the first electron rich molecule they come into contact with, which would be the tetrapeptide in this case.

The effect of pH on the inhibition of hydroxyl radical formation by the tetrapeptide was tested at a tetrapeptide/copper ratio of 2:1. At this ratio, the tetrapeptide gave >95% inhibition of the formation of TBA-reactive species at pH 7.0-8.5. These are physiological pH levels and pH levels that would be expected during ischemia (acidosis occurs in ischemic tissues). At pH 6.0, the tetrapeptide was ineffective at preventing the formation of TBA-reactive species, possibly due to the reduced ability of the histidine to bind copper. The nitrogen atom on the imidazole ring of histidine participates in binding copper with a pKa of 6.0. Therefore, at a pH of 6.0, histidine is only able to bind 50% of the copper. The other 50% of the copper would be unbound or loosely bound to the tetrapeptide by the other amino acids and would, therefore, be able to participate in the production of TBA-reactive species.

Histidine and several peptides with histidine in different positions were tested at 1:1 and 2:1 peptide:copper ratios for their ability to inhibit the production of hydroxyl radicals.

Also, a peptide having an acetylated aspartic acid (Ac-Asp) as the N-terminal amino acid was also tested. The results are presented in Table 3. In Table 3, the % inhibition is the percent decrease in absorbance compared to buffer alone divided by the absorbance of the buffer alone.

As can be seen from the results in Table 3, the peptides with histidine in the second and third positions gave >95% inhibition at a 2:1 peptide:copper ratio, while these peptides at a 1:1 peptide:copper ratio were ineffective. Interestingly, at a 2:1 peptide:copper ratio, the peptide with histidine in the first position and the peptide with acetylated aspartic acid as the N-terminal amino acid provided some protection (about 47% and about 28% inhibition, respectively), although this protection might be attributable to the histidine in the seventh and ninth positions, respectively, ofthese peptides. Histidine alone at a 2:1 histidine:copper ratio provided some protection (about 20% inhibition).

Catalase has been shown to prevent hydroxyl radical formation. Gutteridge and Wilkins, Biochim. Biophys. Acta, 759:38-41 (1983); Facchinetti et al., Cell. Molec. Neurobiol., 18(6):667-682 (1998); Samuni et al., Eur. J Biochem., 137:119-124 (1983). Catalase (0-80 nM) was, therefore, tested in this assay, and it was found to prevent the formation of the pink chromogen (data not shown). This finding suggests that hydrogen peroxide is formed in this assay, since catalase breaks down hydrogen peroxide to water and agrees with Equations 3 and 4 above. Catalase also prevents the formation of the pink chromogen when the L-Asp L-Ala L-His L-Lys [SEQ ID NO:1] tetrapeptide at a tetrapeptide/copper ratio of 1 :1 is present (data not shown). As shown above, at this ratio, the copper is still able to participate in the redox reactions to produce hydroxyl radicals. These experiments show that hydrogen peroxide is an important precursor to the formation of the hydroxyl radical.

TABLE 3

| | Absorbance | Absorbance | % |
|---|---|---|---|
| Compound (Ratio)^{a} | at 532 nm | at 532 nm | Inhibition |
| Copper only (buffer control) | 0.767* | 0 954 | 0 |
| Histindine/copper (2.1) | | 0.760 | 20.3 |
| His Lys Ser Glu Val Ala His Arg Phe | | 0 716 | 24.9 |
| Lys^{b}/copper (1.1) | | | |
| His Lys Ser Glu Val Ala His Arg Phe | | 0 509 | 46.6 |
| Lys^{b}/copper (2:1) | | | |
| Ala His Lys Ser Glu Val Ala His Arg | | 0.843 | 11.6 |
| Phe Lys^{c}/copper (1:1) | | | |
| Ala His Lys Ser Glu Val Ala His Arg | | 0 047 | 95.1 |
| Phe Lys^{c}/copper (2:1) | | | |
| Asp Ala His Lys Ser Glu Val Ala His | 0.645 | | 13.2 |
| Arg Phe Lys^{d}/copper (1:1) | | | |
| Asp Ala His Lys Ser Glu Val Ala His | 0.040 | | 95.8 |
| Arg Phe Lys^{d}/copper (2:1) | | | |
| Ac-Asp Ala His Lys Ser Glu Val Ala | 0.633 | | 16.9 |
| His Arg Phe Lys^{e}/copper (1.1) | | | |
| Ac-Asp Ala His Lys Ser Glu Val Ala | 0.692 | | 27.5 |
| His Arg Phe Lys^{e}/copper (2:1) | | | |
| Asp Ala His Lys^{f}/copper (1:1) | 0.751* | | 1.3 |
| Asp Ala His Lys^{f}/copper (2:1) | 0.029* | | 96.2 |

^{a}All amino acids are L-amino acids.
^{b}SEQ ID NO 5
^{c}SEQ ID NO.4
^{d}SEQ ID NO:3
^{e}SEQ ID NO.6
^{f}SEQ ID NO.1
*Data taken from Table 2 in Example 7.

B. Assay For Superoxide Dismutase (SOD) Activity

The enzyme superoxide dismutase (SOD) is a naturally-occurring enzyme which is responsible for the breakdown in the body of superoxide to hydrogen peroxide (similar to Equation 3). Hydrogen peroxide can then be detoxified by catalase.

SOD was assayed for activity in the assay described in the previous section and was found to have none (data not shown). This result is not surprising since SOD actually converts superoxide radical into hydrogen peroxide. Hydrogen peroxide can then be converted into the hydroxyl radical by reduced copper.

There are reports in the literature that copper complexes have SOD activity. Athar et al., Biochem. Mol. Biol. Int., 39(4):813-821 (1996); Ciuffi et al., Pharmacol Res., 38(4):279-287 (1998); Pogni et al., J. Inorg. Biochem., 73:157-165 (1999); Willingham and Sorenson, Biochem. Biophys. Res. Commun., 150(1):252-258 (1988); Konstantinova et al., Free Rad. Res. Comms., 12-13:215-220 (1991); Goldstein et al., J. Am. Chem. Soc., 112:6489-6492 (1990). This finding is not surprising since SOD itself has copper in its active site.

The SOD activity of copper complexes of the tetrapeptide L-Asp L-Ala L-His L-Lys [SEQ ID NO: 1] was assayed. Superoxide radicals were produced using the xanthine oxidase assay of Beauchamp and Fridovich, Anal. Biochem., 44:276-287 (1971). Xanthine oxidase converts xanthine into uric acid, with oxygen acting as an electron acceptor. This causes superoxide radical to be produced. Superoxide radical is able to reduce nitro blue tetrazolium (NBT). Reduced NBT has a λmax of 560 nm. It is known that copper inhibits xanthine oxidase activity (Konstantinova et al., Free Rad Res Comms., 12-13:215-220 (1991)), so all experiments containing copper also contained ethylenediaminetetracetic acid (EDTA), a known copper chelator. The EDTA-copper complex was tested for SOD activity and was shown to have no SOD activity (data not shown).

To perform the assay for SOD activity, 0.1 mM xanthine (Sigma Chemical Co.), 25 μM NBT (Sigma Chemical Co.), 50 mM sodium carbonate, and 1.2 μM EDTA (Sigma Chemical Co.), were mixed in a cuvette (all final concentrations, final pH 10.2). The reaction was started by the addition of various amounts of a tetrapeptide-copper complex (tetrapeptide/copper ratios of 1:1 and 2:1) and 20 nM xanthine oxidase (Sigma Chemical Co.). The tetrapeptide-copper complex was prepared by mixing the tetrapeptide and copper (as CUCl₂) and allowing the mixture to incubate for 15 minutes at room temperature immediately before addition to the cuvette. The samples were read at time 0 and every 60 seconds for five minutes at 560 nm.

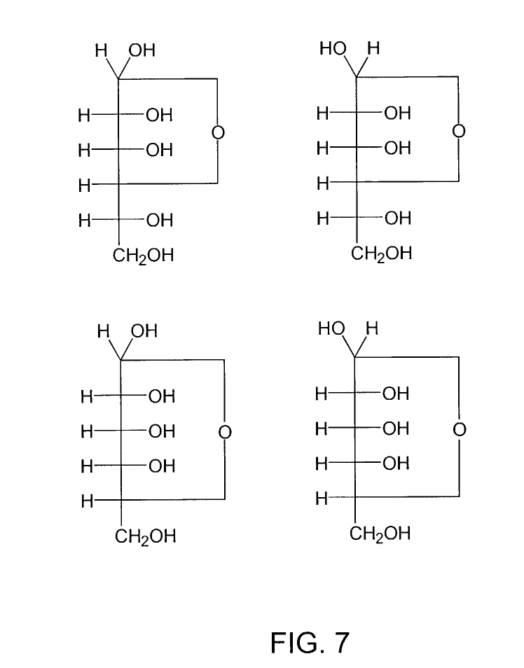

The complex of the tetrapeptide with copper at a ratio of 1:1 was shown to have SOD activity, as evidenced by inhibition of NBT reduction (see FIG. 9). However, the complex was about 500 times less effective than SOD itself, based on IC₅₀ values (amount that gives 50% inhibition) in this assay. The complex of the tetrapeptide with copper at a ratio of 2:1 was found to have no SOD activity (data not shown).

To verify that the 1:1 tetrapeptide-copper complex did not interfere with xanthine oxidase activity, uric acid production was measured at 295 nm. Athar et al., Biochem. Mol. Biol. Int., 39(4):813-821 (1996); Ciuffi et al., Pharmacol Res., 38(4):279-287 (1998). This assay is similar to the SOD assay, except that NBT is not present. Instead, uric acid is assayed at 295 nm every 60 seconds for 5 minutes. It was found that the 1:1 tetrapeptide-copper complex only inhibited uric acid production by 11% at a concentration of 600 nM (data not shown). Therefore, the 1:1 tetrapeptide-copper complex has true SOD activity. Since superoxide is converted to hydrogen peroxide by the complex, this could help to explain why it is not effective at preventing hydroxyl radical production.

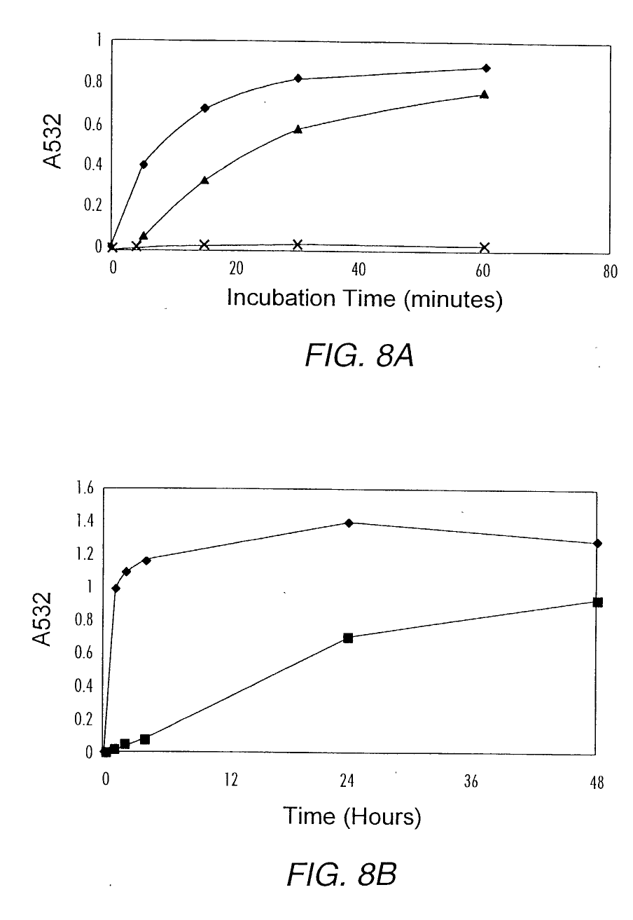

Superoxide radical production was measured in solutions containing the 1:1 or 2:1 tetrapeptide-copper complexes. The assay combined techniques from the TBA assay and the xanthine oxidase assay. NBT was added to all test tubes in order to quantitate its reduction by superoxide radical. The samples also contained ascorbate and copper and were incubated at 37° C. At 5, 15, 30 and 60 minutes, the samples were removed from the incubator and read at 560 nm. The results are shown in FIG. 10. In the sample containing the 2:1 tetrapeptide-copper complex, NBT reduction increased over time and reached a maximum at 30 minutes. The sample containing the 1:1 tetrapeptide-copper complex also showed an increase in NBT reduction, with a decreased maximum reached at 60 minutes. These data suggest that superoxide accumulates in the sample containing the 2:1 tetrapeptide-copper complex, while the 1:1 tetrapeptide-copper complex mimics superoxide dismutase.

The likely sequence of events that occurs in the production of hydroxyl radicals is as follows:

O₂→O₂^{.-}→H₂O₂→OH^{.} (Eq. 5).

It has already been shown that the 1:1 tetrapeptide-copper complex can convert superoxide radical (O₂^{.-}) into hydrogen peroxide (H₂O₂). This is the SOD activity of the complex. The 2:1 tetrapeptide-copper complex cannot facilitate this conversion since the two molecules of the tetrapeptide fill all six coordination bonds of copper. This explains why the 2:1 tetrapeptide-copper complex is so effective because it inhibits the formation of hydrogen peroxide, which could in turn react with reduced copper to produce hydroxyl radicals via the Fenton reaction. The 1:1 tetrapeptide-copper complex also provides a valuable service by eliminating the superoxide radical. Even though it produces hydrogen peroxide, most compartments of the human body have sufficient quantities of the enzyme catalase that can eliminate hydrogen peroxide. In the brain, however, catalase activity is reported to be minimal. Halliwell et al., Methods in Enzymol., 186:1-85 (1990). Therefore, the brain is a particularly vulnerable organ during periods of ischemia, since copper is released due to the acidosis that accompanies ischemia.

C. Protection of DNA

DNA strand breaks were measured according to the method of Asaumi et al., Biochem. Mol Biol. Int., 39(1):77-86 (1996). Briefly, 17 μg/ml of plasmid pBR322 DNA was allowed to pre-incubate for 15 minutes at room temperature with 50 μM CuCl₂ and concentrations of the tetrapeptide of 0-200 μM. Then, 2.5 mM ascorbate was added to each reaction, and the mixture was incubated for 1 hour at 37° C. The total volume of the mixture was 16 μL. Next, 3 μL of loading buffer containing 0.25% (w/v) bromophenol blue, 0.25% (w/v) xylene cyanole FF, and 40% (w/v) sucrose in water was added. The samples were separated by electrophoresis in a 0.8% agarose gel for 90 minutes at 70 Volts. The gel was stained in 1X TBE (Tris-Borate-EDTA buffer) containing 2 μg/ml ethidium bromide for 30 minutes. The gel was then destained in 1X TBE for 5 minutes prior to photographing the gel.

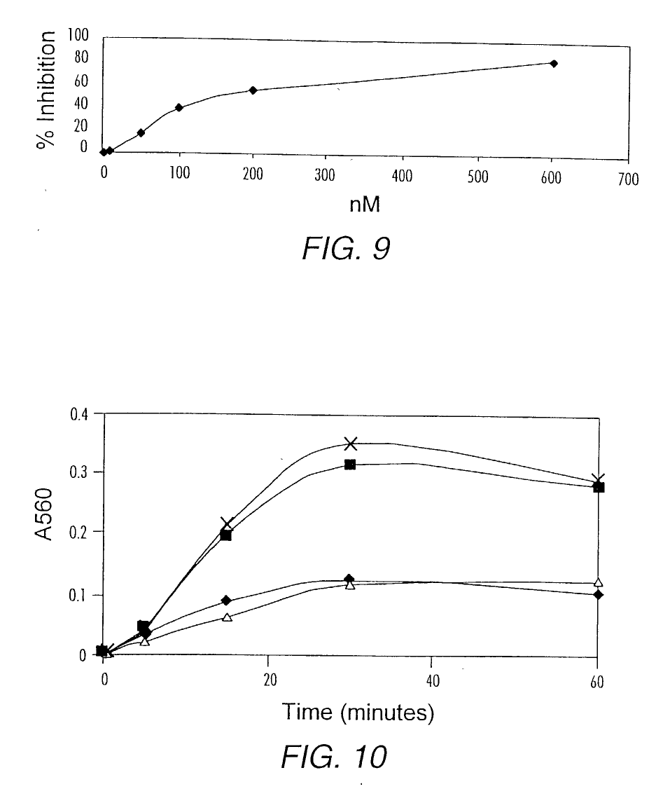

The results showed that the tetrapeptide was very effective at preventing the formation of DNA strand breaks. See FIG. 11. Optimal protective tetrapeptide:copper ratios were 2:1 and greater, since superhelical circular DNA was still visible on the gel at these ratios. At a tetrapeptide:copper ratios of 1:1 or less, nicked circular DNA, linear DNA and more damaged DNA (smears) were visible.

### EXAMPLE 9

Reduction of the Damage Done to DNA by ROS

ROS damages DNA by causing strand breaks, base modifications, point mutations, altered methylation patterns, and DNA-protein cross linking (Marnett, Carcinogenesis 21:361-370 (2000); Cerda et al., Mutat. Res. 386:141-152 (1997)). Copper, iron, and other transition metals, in the presence of reducing agents, catalyze the production of ROS such as superoxide (O₂•), hydrogen peroxide (H₂O₂) and the hydroxyl radical (OH•) through both the Haber-Weiss and Fenton reactions (Stoewe et al., Free Radic. Biol. Med. 3:97-105 (1987)). OH• is considered the most reactive and damaging ROS and is capable of producing all the above DNA lesions (Marnett, Carcinogenesis 21:361-370 (2000)). Previous investigations have reported that OH• induced, single- and double-strand DNA breaks occur during site-specific copper ion reactions in vitro and during excessive copper exposure in vivo (Chiu et al., Biochemistry 34:2653-2661 (1995); Kim et al., Free Radic. Res. 33:81-89 (2000); Hayashi et al., Biochem. Biophys. Res. Comm. 276:174-178, doi:10. 1006/bbrc.2000.3454 (2000)).

Telomeres, which are repeats of the hexanucleotide TTAGGG, exist at the ends of DNA to form a “protective cap” against degradation, chromosomal rearrangement, and allow the replication of DNA without the loss of genetic information (Reddel, Carcinogenesis 21:477-484 (2000)). The classical theory of cellular aging, or senescence, involves the telomere end replication problem (Olovnikov, J. Theor Biol. 41:181-190 (1973)). DNA polymerase is unable to replicate the terminal end of the lagging strand during DNA replication resulting in the loss of 30-500 base pairs (Harley et al., Nature 345:458-460 (1990); von Zglinicki et al., Exp. Cell Res. 220:186-193, doi:10.1006/excr.1995.1305 (1995)). Somatic cells are unable to replace these lost telomeric repeats, leading to progressive telomere shortening during a cell's replicative life. Senescence is manifested when telomere length reaches a critical threshold (Reddel, Carcinogenesis 21:477-484 (2000)). Premature senescence has been documented in human fibroblasts exposed to oxidative stress (Chen et al., Proc. Natl. Acad. Sci. USA 91:4130-4134 (1994)). Examination of telomere length in fibroblasts after several population doublings under conditions of higher oxidative stress reveals shortened telomere lengths similar to senescence under normal conditions (von Zglinicki et al., Exp. Cell Res. 220:186-193, doi: 10.1006/excr.1995.1305 (1995)). These data suggest that ROS-induced DNA damage in the telomere sequence may play an important role in telomere shortening.

In this study, the ability of Asp Ala His Lys [SEQ ID NO:1] to protect DNA and telomeres from ROS damage induced by copper coupled with ascorbic acid was examined.

A. Materials and Methods

Reagents. The synthetic D-analog of Asp Ala His Lys (D-Asp Ala His Lys) was obtained from Bowman Research Ltd. (Newport, Wales, UK). TeloTAGG Telomere Length Assay and X-ray film were purchased from Roche Molecular Biochemicals (Mannheim, Germany). DNeasy genomic isolation kits were purchased from Qiagen (Valencia, Calif.). Hybond-N+ nylon membrane was ordered from Amersham Pharmacia Biotech (Piscataway, N.J.). All other chemicals were obtained from Sigma (St. Louis, Minn.).

DNA treatments. DNA strand breaks were measured using a modified method of Asaumi (Asaumi et al., Biochem. Mol. Biol. Int. 39:77-86 (1996)). Raji cells, a Burkitt lymphoma derived cell line (obtained from American Type Culture Collection (ATCC), Rockville, Md., ascension number CCL-86), were grown in Iscove's modified Dulbecco's medium (IMDM) with 10% fetal calf serum (FCS) at 10% CO₂ and 37° C. Genomic DNA was isolated using DNeasy spin columns (Qiagen) following the manufacturer's protocol. Then, 1 g genomic DNA was incubated per reaction with CuCl₂, ascorbic acid, and/or the tetrapeptide in 10 mM sodium phosphate buffer, pH 7.4. Final concentrations were as follows: CUCl₂=10 μM, 25 μM, and 50 μM; ascorbic acid=25 μM, 50 μM, and 100 μM; D-Asp Ala His Lys=50 μM, 100 μM, and 200 μM. Total reaction volumes of 20 μl in 0.2 ml PCR tubes were incubated at 37° C. for 2 hours. Following the incubation, strand breaks were visualized by immediately adding 5 μl of loading dye [0.25% (w/v) bromophenol blue and 40% (w/v) sucrose] and loading on a 0.5% tris acetic acid EDTA (TAE) agarose gel. Gels were then run at 70V for 90 min and stained using 2 μg/ml ethidium bromide for 30 minutes. Prior to photographing, gels were rinsed in TAE for 10 minutes.

Cell treatments: Raji cells were washed with PBS (10 mM phosphate buffered saline; 138 mM NaCl; 2.7 mM KCl pH 7.4). Then, 1.5×10⁶ cells were put into 5 ml PBS containing CuCl₂, ascorbic acid, and/or D-Asp Ala His Lys. Final concentrations were as follows: CuCl₂=10 μM, 25 μM, and 50 μM; ascorbic acid=100 μM, 250 μM, and 500 μM; D-Asp Ala His Lys=50 μM, 100 μM, and 200 μM. The cells were then incubated at 37° C. for 2 hours. Following the incubation, genomic DNA was isolated using DNeasy columns. DNA damage was visualized by 0.5% TAE agarose gel electrophoresis.

Telomere Length Assay: To examine telomere damage, the TeloTAGG Telomere Length Assay (Roche) was used according to manufacturer's recommendations: digesting 1 μg of genomic DNA per reaction using HinfI and RSA I. Samples were then run on a 0.8% TAE agarose gel at 70V for 2 hours. Southern blots were performed and probed using a digoxigenin (DIG) labeled telomere specific oligonucleotide. For cell treated samples, genomic DNA was used as described above. For DNA treated samples, reactions were setup as above, brought to 200 μl with PBS, and isolated using DNeasy columns prior to restriction digestions.

B. Results and Discussion

Copper ions, an essential part of chromatin (Dijkwel et al., J. Cell Sci. 84:53-67 (1986)), are present within DNA (Wacker et al., J. Biol. Chem. 234:3257-3262 (1959)) and may participate in oxidative DNA damage (Chin et al., Biochemistry 34:2653-2661 (1995); Hayashi et al., Biochem. Biophys. Res. Comm. 276:174-178, doi:10.1006/bbrc.2000.3454 (2000); Kagawa et al., J. Biol. Chem. 266:20175-20184 (1991)). In the presence of ascorbate or other reducing agents, copper can lead to the production of ROS by catalyzing the following reactions (Biaglow et al., Free Radic. Biol. Med. 22:1129-1138 (1997)):

1) 2 Cu₂²⁺+ascorbate→2 Cu⁺+dehydroascorbate+2H⁺

2) Cu⁺+O₂→O₂.+Cu²⁺

3) Cu⁺+O₂.+2H⁺→Cu²⁺+H₂O₂

4) Cu⁺+H₂O₂→OH⁻+OH•+Cu²⁺

While iron is found at higher concentrations physiologically, oxidation by copper and H₂O₂ is 50 times faster than iron (Stoewe et al., Free Radic. Biol. Med. 3:97-105 (1987); Halliwell J. Neurochem. 59:1609-1623 (1992)). Due to the negative charge of the sugar phosphate backbone, cations can loosely bind DNA. Site-specific binding of copper ions within base pairs may be important to the regulation of DNA biosynthesis (Minchenkova et al., Biopolymers 5:615-625 (1967)). Unlike iron-catalyzed reactions, OH• scavengers do not prevent copper-mediated oxidative damage suggesting that oxidative DNA damage occurs in close proximity to the copper ions (Oikawa et al., Biochim. Biophys. Acta 1399:19-30 (1998)). The reactivity of OH• is so great that, presumably, OH• interactions only occur at or near the site of OH• production (Marnett, Carcinogenesis, 21, 361-370 (2000)). Oikawa, et. al., (Oikawa et al., Biochim. Biophys. Acta 1399:19-30 (1998)) have shown that the following copper-mediated ROS reaction also occurs, and that the resulting DNA-copper-peroxide complex may be even more damaging to DNA than OH•:

Cu⁺+H₂O₂→Cu⁺OOH+H⁺

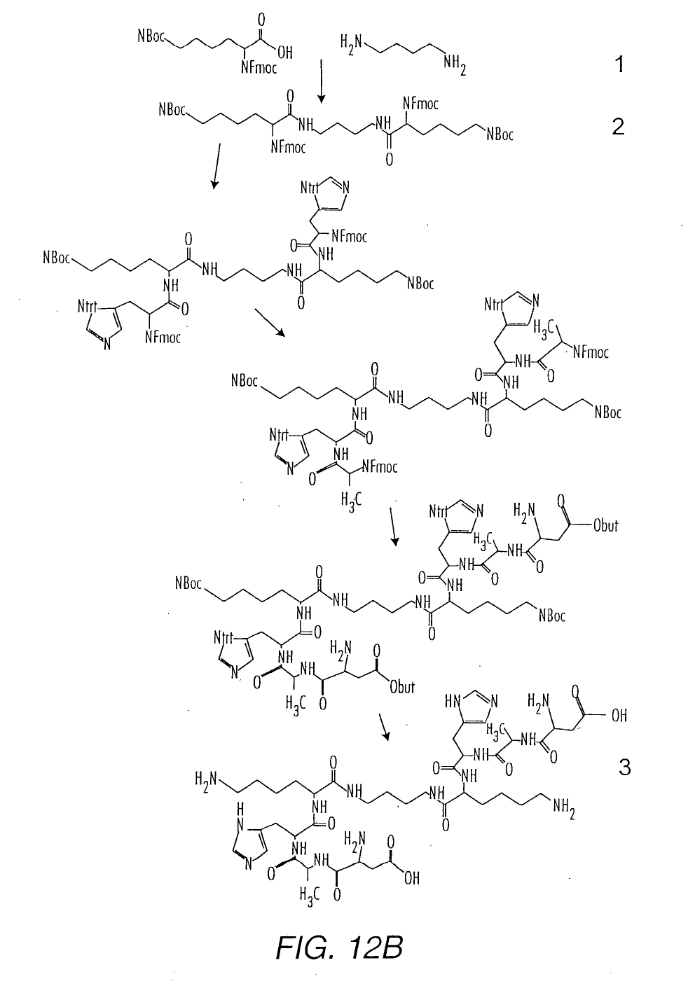

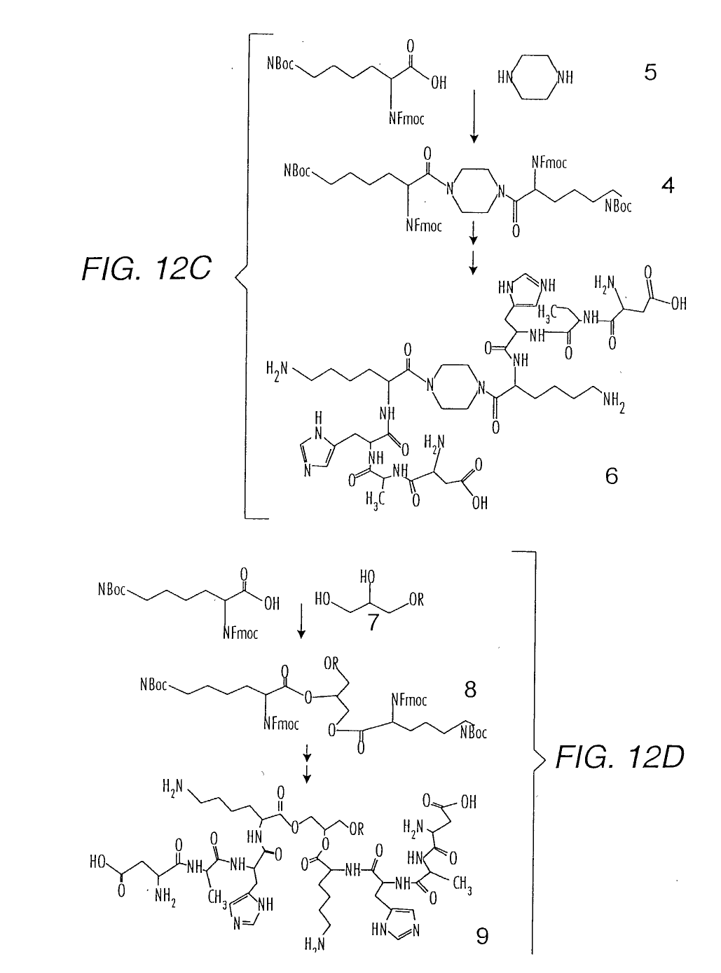

As expected, the results of the above-described experiments showed that copper and ascorbic acid alone were unable to cause strand breaks. When CuCl₂ and ascorbic acid were combined, a dose dependent accumulation of lower molecular weight DNA fragments was seen, the result of double strand breaks. These double strand breaks were attenuated by D-Asp Ala His Lys in a dose dependent manner (FIG. 13). At molar ratios of 1:1 (50 μM copper to 50 μM D-Asp Ala His Lys) and 1:2, some strand breaks were apparent. By elevating the ratio to 1:4, no strand breaks were detected. Similar results were observed in Raji cells treated with copper and ascorbic acid (FIG. 14). A lower ratio of 1:2 (copper to D-Asp Ala His Lys) provided complete protection to DNA in cell samples. It is reasonable to expect that DNA samples would require higher D-Asp Ala His Lys levels due to competition for copper with DNA and proximal OH• attack. The separation of DNA and copper would be critical in these samples necessitating the need for elevated D-Asp Ala His Lys. In cell samples, damage would be attributable to H₂O₂.H₂O₂ is freely diffusible, can penetrate to the nucleus, and has been shown to damage DNA in fibroblasts (Chen et al., Proc. Natl. Acad Sci. USA 91:4130-4134 (1994); von Zglinicki et al., Free Radic. Biol Med. 28:64-74 (2000)). Entrance of H₂O₂ into the cell may lead either to the formation of DNA peroxide complexes with native metals or to the release of sequestered metal stores that, combined with endogenous reducing agents (reduced glutathione (GSH), reduced nicotinamide dinucleotide (NADH), and ascorbic acid), would drive the production of OH•. One possible mechanism of D-Asp Ala His Lys protection would be the chelation of copper ions, thereby preventing production of OH• and H₂O₂. Another mode of protection may be the formation of D-Asp Ala His Lys-copper-peroxide complexes which would absorb the OH• damage rather than DNA, “mop-up” peroxides, and perhaps, in cell samples, keep H₂O₂ outside the cell.

Prior reports suggest that oxidative DNA damage may be directed at G-C rich areas, including telomeres. Rodriguez, et. al., reported that copper induced ROS damage primarily targeted DNA guanine (Rodriguez et al., Cancer Res. 57:2394-2403 (1997)). Strong, preferential binding of Cu (II) to the G-C pair has been reported at the N-7 and O-6 of the guanine bases plus the N-3 of cytosine (Kagawa et al., J. Biol. Chem. 266:20175-20184 (1991)). DNA peroxides complexes formed at these positions are believed to direct OH• attack to adjacent bases (Oikawa et al., Biochim. Biophys. Acta 1399:19-30 (1998)). In addition, GGG in telomeric DNA has been shown to be sensitive to copper mediated ROS damage (Oikawa et al., FEBS Lett 453:365-368 (1999)).

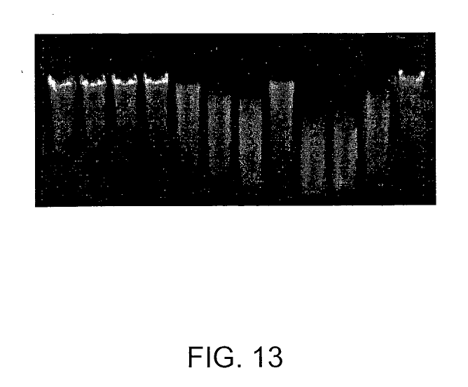

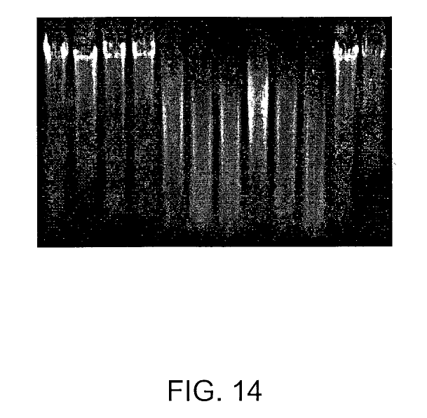

Examination of the telomere in the genomic DNA samples in the present study showed double strand breaks in response to oxidative stress. DNA samples examined by Southern blot showed severely depleted and shortened telomere sequences (FIG. 15). Cell treatments showed damage to the telomere with some conservation of the sequence, even at the highest levels of copper and ascorbic acid used (FIG. 16), which may be attributed to ROS production outside the cells with the DNA sheltered inside the nucleus. D-Asp Ala His Lys protected the telomere from copper-mediated damage in these samples.

In addition to the double strand breaks detected in the experiments, other DNA lesions may be involved in ROS disease processes. Some cations, including copper, bound loosely to the phosphate backbone have been implicated in strand breaks while those coordinated in the helix cause base modifications (Marnett, Carcinogenesis 21:361-370 (2000); Rodriguez et al., Cancer Res. 57:2394-2403 (1997)). Episodes of increased copper and oxidative stress may direct DNA damage to G-C rich areas. In addition to telomeres, G-C rich areas exist at the 5′ end of many genes (Bird, Nature 321:209-213 (1986)) hinting toward a site of oxidative damage involved in gene regulation. 8-Oxo-deoxyguanosine (8-oxo-dG) is a common DNA adduct produced by ROS, which can result in G→T point mutations widely seen in mutated oncogenes (Mamett, Carcinogenesis 21:361-370 (2000)). Conditions such as acidosis occurring during myocardial ischemia or alterations of ceruloplasmin have been shown to mobilize free copper to catalyze local oxidative tissue and DNA damage (Kim et al., Free Radic. Res. 33:81-89 (2000); Chevion et al., Proc. Natl. Acad. Sci. USA 90:1102-1106 (1993)). Levels of 8-oxo-dG are reported to be three to four times higher in the DNA of ischemic rat hearts than in controls (You et al., J. Mol. Cell Cardiol. 32:1053-1059, doi:10.1006/jmcc.2000.1142 (2000)). In addition, chronic inflammation can produce areas of localized oxidative damage. Inflammatory cells, such as macrophages and neutrophils, release ROS that have been shown to damage the DNA of nearby cells (Shacter et al., Carcinogenesis 9:2297-2304 (1988)). Nitric oxide and superoxide released from activated leukocytes can lead to the production of peroxynitrite, which is more reactive with 8-oxo-dG than unmodified bases and possibly exacerbates the damage (Marnett, Carcinogenesis 21:361-370 (2000)).

C. Summary

Both DNA and the telomeric sequence are susceptible to copper-mediated ROS damage, particularly damage attributedto hydroxyl radicals. In this study, ROS-induced DNA double strand breaks and telomere shortening were produced by exposure to copper and ascorbic acid. D-Asp-Ala-His-Lys, a copper chelating tetrapeptide D-analog of the N-terminus of human albumin, attenuated DNA strand breaks in a dose dependent manner. The D-tetrapeptide, at a ratio of 4:1 (peptide:Cu), provided complete protection of isolated DNA and, at a ratio of 2:1 (peptide:Cu), completely protected Raji Burkitt cells' DNA exposed to copper/ascorbate. Southern blots of DNA treated with copper/ascorbate showed severe depletion and shortening of telomeres with some conservation of telomere sequences. The D-tetrapeptide provided complete telomere length protection at a ratio of 2:1 (peptide:Cu). While the exact mechanisms for ROS DNA damage have yet to be fully elucidated, D-Asp Ala His Lys inhibited copper-induced DNA double-strand breaks by ROS in both genomic DNA and in the telomere sequence.

### EXAMPLE 10

Inhibition Of IL-8 Release

Interleukin 8 (IL-8) is a pro-inflammatory cytokine and a potent chemoattractant and activator of neutrophils. It has also been reported to be a chemoattractant and activator of T-lymphocytes and eosinophils. IL-8 is produced by immune cells (including lymphocytes, neutrophils, monocytes and macrophages), fibroblasts and epithelial cells. Reports indicate an important role for IL-8 in the pathogenesis of respiratory viral infections, asthma, bronchitis, emphysema, cystic fibrosis, acute respiratory distress syndrome, sepsis, multiple organ dysfunction syndrome, and other inflammatory disorders.

The IL-8 release by Jurkat cells (American Type Culture Collection (ATCC), Rockville, Md.) exposed to copper and ascorbic acid (to produce ROS—see Examples 7, 8 and 9) was investigated. To do so, 1×10⁶ Jurkat cells were incubated at 37° C. and 5% CO₂ in 0.5 ml IMDM medium (ATCC) (serum-free) with insulin transferin selenite solution (ITSS; Sigma) for 24 hours with the following additives.

Experiment 1:

a. None (control);

b. Asp Ala His Lys [SEQ ID NO: 1] (“DAHK”)-200 μM and ascorbic acid-500 μM;

c. CuCl₂-10 μM and ascorbic acid-500 μM;

d. CuCl₂-25 μM and ascorbic acid-500 μM;

e. CuCl₂-50 μM and ascorbic acid-500 μM;

f. CuCl₂-100 μM and ascorbic acid-500 μM;

g. CuCl₂-50 μM and DAHK-50 μM and ascorbic acid-500 μM;

h. CuCl₂-50 μM and DAHK-100 μM and ascorbic acid-500 μM; and

i. CuCl₂-50 μM and DAHK-200 μM and ascorbic acid-500 μM.

Experiment 2:

a. None (control);

b. CuCl₂-100 μM;

c. DAHK-200 μM and ascorbic acid-500 μM;

d. CuCl₂-25 μM and ascorbic acid-500 μM;

e. CuCl₂-50 μM and ascorbic acid-500 μM;

f. CuCl₂-100 μM and ascorbic acid-500 μM;

g. CuCl₂-50 μM and DAHK-50 μM and ascorbic acid-500 μM;

h. CuCl₂-50 μM and DAHK-100 μM and ascorbic acid-500 μM; and

i. CuCl₂-50 μM and DAHK-200 μM and ascorbic acid-500 μM.

Experiment 3:

a. None (control);

b. CuCl₂-100 μM;

c. DAHK-400 μM and ascorbic acid-250 μM;

d. CuCl₂-25 μM and ascorbic acid-250 μM;

e. CuCl₂-50 μM and ascorbic acid-250 μM;

f. CuCl₂-100 μM and ascorbic acid-250 μM;

h. CuCl₂-100 μM and DAHK-200 μM and ascorbic acid-250 μM; and

i. CuCl₂-100 μM and DAHK-400 μM and ascorbic acid-250 μM.

After the 24-hour incubation, supernatants were collected and the concentration of IL-8 in each supernatant was determined by an ELISA using human IL-8 matched pair antibodies (Endogen, Cambridge, Mass.). The ELISA was performed using an ELISA kit from Endogen, Cambridge, Mass. according to the manufacturer's instructions with the following exceptions: (1) coating antibody at 1 μg/ml; (2) detecting antibody 30 ng/ml; StrepAvidin HRP diluted 1:32,000.

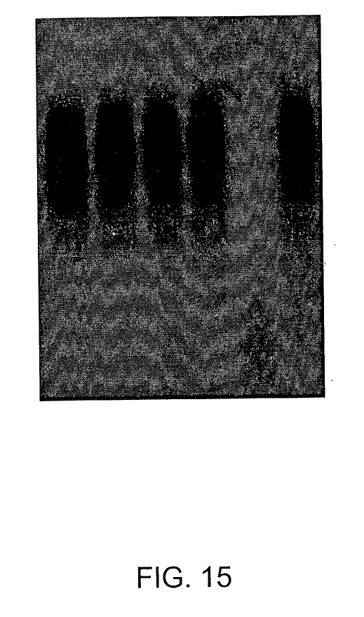

The results are presented in FIG. 17A (Experiment 1), FIG. 17B (Experiment 2), and FIG. 17C (Experiment 3). As can be seen, copper and ascorbic acid caused the release of IL-8 from the cells in a dose-dependent manner. As can also be seen, DAHK inhibited the release of IL-8, with the best results being obtained with an 8:1 DAHK:Cu ratio.

### EXAMPLE 11

Inhibition Of Oxidation Of CoA

Coenzyme A (CoA) is essential for acetylation reactions in the body and, as a consequence, plays a critical role in the metabolism of carbohydrates and fatty acids. CoA can be oxidized to a disulfide which cannot participate in acetylation reactions. As a result, metabolism and energy utilization are inhibited.

In this example, it was investigated whether Cu(II) could oxidize CoA and, if so, whether the tetrapeptide Asp Ala His Lys [SEQ ID NO: 1] (Bowman Research, Inc., United Kingdom) could protect CoA (Sigma) from oxidation by Cu(II). The experimental setup and results are presented in Table 4 below. All of the ingredients were added simultaneously and, after a 15-minute incubation, absorbance at 412 nm (A412) was measured. Free thiol groups were measured using DTNB. DTNB is dithionitrobenzoic acid (Sigma).

As can be seen from Table 4, Cu(II) oxidized CoA. As can also be seen, the tetrapeptide at a 1:1 tetrapeptide:Cu(II) ratio provided some protection of CoA, and the tetrapeptide at a 2:1 tetrapeptide:Cu(II) ratio provided 100% protection.

TABLE 4

| | 1 | 2 | 3 | 4 | 5 | 6 | 7 |
|---|---|---|---|---|---|---|---|
| Asp Ala | 50 μl | 50 μl | | | 50 μl | 50 μl | 50 μl |
| His Lys | (190 | (190 | | | (190 | (190 | (190 |
| (2 mM) | μM) | μM) | | | μM) | μM) | μM) |
| CoA(2 | 50 μl | 50 μl | 50 μl | 50 μl | | | 50 μl |
| mM) | (190 | (190 | (190 | (190 | | | (190 |
| | μM) | μM) | μM) | μM) | | | μM) |
| CuCl₂ (1 | 100 μl | | 100 μl | | | 100 μl | 50 μl |
| mM) | (190 | | (190 | | | (190 | (85 μM) |
| | μM) | | μM) | | | μM) | |
| Tris | 200 μl | 300 μl | 250 μl | 350 μl | 350 μl | 250 μl | 250 μl |
| buffer, | | | | | | | |
| 50 mM, | | | | | | | |
| pH 8.0 | | | | | | | |
| DTNB | 125 μl | 125 μl | 125 μl | 125 μl | 125 μl | 125 μl | 125 μl |
| (3 mM) | | | | | | | |
| A412 | 0.279 | 1.119 | 0.127 | 0.888 | 0.142 | 0.113 | 1.111 |

### EXAMPLE 12

Inhibition Of IL-8 Secretion By d-DAHK

Systemic inflammatory response syndrome (SIRS) can occur following severe trauma, sepsis, or major surgery and frequently progresses to multiple organ failure, the most common cause of death in surgical intensive care units. Vascular endothelial cells lining blood vessels have been shown to adversely contribute to early SIRS by secreting excessive amounts of interleukin-8 (IL-8), a potent pro-inflammatory cytokine associated with an increased risk of multiple organ failure and death after severe trauma. McGill et al., World J. Surg; . 22, 171 (1998); Patrick et al., Am. J. Surg., 172, 425 (1996). Interestingly, endogenous copper is reported to play a central role in post-ischemic reperfusion injury (Powell et al., Am. J. Physiol. 277(3 Pt 2), H956 (1999)), which is also associated with increased IL-8 levels and endothelial dysfunction. However, the role of copper in activating IL-8 secretion from human endothelial cells has not previously been identified and may be important in the pathogenesis of SIRS and multiple organ failure.

This example presents data showing for the first time that endothelial cells secrete markedly elevated levels of IL-8 after exposure to a physiologically relevant concentration of copper. Further, addition of a high-affinity Cu(II)-binding peptide significantly inhibits copper-induced IL-8 secretion from endothelial cells.

Copper is an essential human trace element that is closely regulated by plasma proteins, such as ceruloplasmin and albumin, during homeostatic conditions and normal pH. Major trauma or sepsis can lower the pH and produce microvascular and tissue acidosis due to increased tissue oxygen requirements, impaired oxygen extraction, maldistributed blood flow, and diminished energy stores. Mizock et al., Crit. Care Med. 20, 80 (1992). The acidic environment subsequently allows Cu(II) ions to be released from carrier proteins (Lamb et al., FEBS Lett. 338, 122 (1994)) and to be free to participate in various biochemical pathways such as oxidative stress, inactivation of activated protein C, and inhibition of endothelial nitric-oxide synthase. Bar-Or, et al., Biochem. Biophys. Res. Commun. 290, 1388 (2002); Bianchini et al., J. Biol. Chem. 274, 20265 (1999).

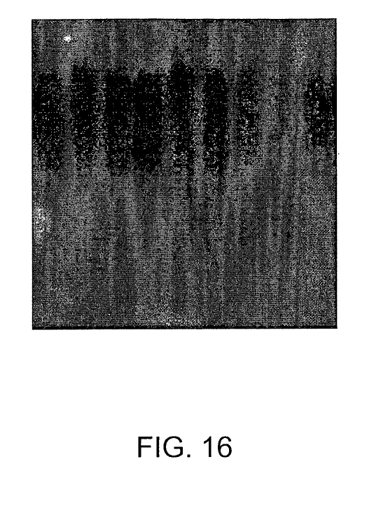

Human umbilical vein endothelial cells (HUVEC) (5.0×10⁴ cells) were incubated in serum-free and ascorbate-free endothelial cell basal medium-2 (EGM₂) medium (BioWhittaker) with ITS supplement with (i) copper, (ii) a tetrapeptide analogue of the high-affinity, N-terminal Cu(II) binding site of human albumin (D-Asp D-Ala D-His D-Lys or d-DAHK), or (iii) both of them (n=3, in duplicate). IL-8 was determined by ELISA (see Example 10). HUVEC incubated for 24 hours with 25 μM CuCl₂ showed a >5.5-fold higher IL-8 secretion as compared to controls incubated with water (P<0.007, t test) (FIG. 25). The tetrapeptide d-DAHK at 1:1 and 2:1 molar ratios (d-DAHK:copper) inhibited IL-8 secretion by 86.1% (P=0.007) and 102.4% (P=0.002), respectively, after 24 hours of incubation (FIG. 18). Decreased IL-8 secretion by 100 μM d-DAHK alone after 24 hours of incubation compared to controls was not significant (P=0.16) (FIG. 18). Upon visual examination, all cells appeared to be viable at 24 hours. Preliminary experiments with both human lung microvascular and human iliac artery endothelial cells demonstrated similar results after exposure to copper and d-DAHK. Additional HUVEC data showed no copper-induced secretion of tumor necrosis factor-α (TNF-α), prostaglandin E₂ or prostacyclin and no increase in IL-8 levels after three hours exposure to copper (data not shown). The latter result indicates that the copper-induced increase in IL-8 results from synthesis of the IL-8, rather than release of IL-8 from pre-existing storage sites.

Analysis of the culture medium after exposure to copper and d-DAHK, alone and together, but without any cells, detected primarily Cu(II) and <7% Cu(I). This was determined as follows. Cupric(II) chloride (10-50 μM) and d-DAHK (6.25-100 μM) were incubated alone and together in EGM₂ medium for 24 hours with 5% CO₂, then filtered and combined with 400 μM bicinchoninic acid for 1 hour (all at 37° C.). Cuprous(I) chloride standards (0.5-50 μM) were made in water from 1 mM CuCl stock containing 20 mM ascorbate to insure predominance of Cu(I). Cu(I) was read at 562 nm in duplicate (Shimadzu spectrophotometer, Model UV160U).

Cu(I) ions catalyze the generation of reactive oxygen species resulting in IL-8 secretion from other cell types. However, the results presented here provide evidence that Cu(II) ions stimulate IL-8 secretion from human endothelial cells independent of oxidative stress. In-addition, a high-affinity Cu(II)-binding compound significantly inhibited copper-induced endothelial cell IL-8 secretion. These data suggest that sequestration of unbound Cu(II) ions could have human therapeutic potential.

A possible mechanism for the Cu(II)-induced endothelial IL-8 secretion may be activation of serine-threonine kinase Akt (protein kinase B), which has been reported in human fibroblasts. Ostrakhovitch et al., Arch. Biochem. Biophys. 397, 232 (2002). If a similar pathway is stimulated in human endotheliium in vivo, copper could be a major contributor in the development of systemic inflammation by activating nuclear factor-kappaB (NF-kappaB). NF-kappaB is an inflammation transcription factor well known to stimulate high levels of cytokines that significantly augment vascular and cellular inflammatory responses. Additionally, it is possible that sustained or recurring post-ischemic reperfusion injury and acidosis in the hours after the initial injury could result in persistent Cu(II)-induced IL-8 secretion.

It should also be noted that there is evidence that Asp Ala His Lys [SEQ ID NO:1] is degraded in vivo to produce Asp Ala diketopiperazine (DA-DKP) (data not shown). DA-DKP has been shown to be anti-inflammatory (see U.S. patent application Ser. No. 09/922,234, filed Aug. 2, 2001, and PCT application WO 02/11676, the complete disclosures of which are incorporated herein by reference.), and Asp Ala His Lys [SEQ ID NO: 1] may cause additional anti-inflammatory effects as a result of being degraded to produce DA-DKP.

### EXAMPLE 13

Inhibition Of IL-8 By Asp Ala His Lys in Crevicular Gingival Fluid

Cervicular gingival fluid (CGF) was obtained from normal controls (II individuals) and patients with gingivitis (7 individuals) and periodontitis (9 individuals).

Crest Whitestrips™ were applied to the teeth of 5 of the normal controls according to the package instructions. CGF samples were obtained at various times after the Crest Whitestrips™ were applied to the teeth, as indicated below.

In a separate experiment, a 4 mM solution of the tetrapeptide L-Asp L-Ala L-His L-Lys [SEQ ID NO: 1] (referred to herein as DAHK-1199) was prepared in phosphate buffered saline (0.1 M sodium phosphate and 0.15 M sodium chloride, pH 7.2), and the pH was adjusted to 7.4. Then 0.1 ml of this solution of DAHK was applied evenly over the surface of each Crest Whitestrip™. The Crest Whitestrips™ were then applied to the teeth of 3 of the normal controls according to the package instructions. CGF samples were obtained before the Crest Whitestrips™ with the DAHK-1199 were applied to the teeth and at various times thereafter as indicated below.

CGF was collected using dental wicks (Sigma, St. Louis, Mo.). The dental wicks containing the CGF were placed in 150 μl storage buffer (phosphate buffered saline (0.1 M sodium phosphate and 0.15 M sodium chloride, pH 7.2) containing 4% bovine serum albumin (Sigma, St. Louis, Mo.), 0.1 mg/ml phenylmethylsulfonyl fluoride (PMSF; Sigma, St. Louis, Mo.), and 0.1 mg/ml aprotinin (Sigma, St. Louis, Mo.) and frozen at −20° C. until used.

The frozen CGF samples were thawed on ice and kept at 4° C. Then ELISA assays were performed to determine the amounts of interleukin 8 (IL-8), tumor necrosis factor-α (TNFα), and soluble tumor necrosis factor-α receptor (sTNFR75). Not enough CGF was obtained to perform all of the ELISA assays on all of the samples; the number of samples assayed will be indicated below.

The IL-8 ELISA was performed as follows. Anti-human IL-8 antibody (Pierce Endogen, Rockford, Ill.; catalogue number M801-E, lot number CK41959) was diluted to 1 μg/ml in phosphate buffered saline, pH 7.2-7.4, and 100 μl of the diluted antibody was added to each well of Nunc Maxisorb ELISA strip plates. The plates were incubated overnight at room temperature. The liquid was aspirated from the wells, and the plates were blotted on a paper towel. Then, 200 μl of assay buffer (phosphate buffered saline, pH 7.2-7.4, containing 4% bovine serum albumin (Sigma, St. Louis, Mo.; ELIS grade=low fatty acid and IgG)) were added to each well, and the plates were incubated for 1 hour at room temperature. The liquid was aspirated from the wells, and the wells were washed 3 times with wash buffer (50 mM Tris, 0.2% Tween-20, pH 7.9-8.1) and were then blotted on a paper towel. Standards and CGF samples (50 μl/well; standards were diluted in storage buffer) were added to the wells, and the plates were incubated for 1 hour at room temperature with gentle shaking. The liquid was aspirated, the wells were washed 3 times with wash buffer, and the plates were then blotted on a paper towel. Then, 100 μl of biotin-labeled anti-human IL-8 (Pierce Endogen, Rockford, Ill.; catalogue number M802-E, lot number CE49513), diluted to 60 ng/ml in assay buffer, were added to each well. The plates were incubated for 1 hour at room temperature, the liquid was aspirated, the wells were washed 3 times with wash buffer, and the plates were blotted on a paper towel. Then, 100 μl of HRP-conjugated streptavidin (Pierce Endogen, Rockford, Ill.; catalogue number N100) in assay buffer, were added to each well. The plates were incubated for 30 minutes at room temperature, the liquid was aspirated, the wells were washed 3 times with wash buffer, and the plates were blotted on a paper towel. Finally, 100 μl of TMB substrate solution (Pierce Endogen, Rockford, Ill.; catalogue number N301) were added to each well. The plates were incubated for 30 minutes at room temperature. The reaction was stopped by adding 100 μl/well of 0.18 M H₂SO₄. The optical densities at 450 nm and 530 nm were read on an ELISA plate reader and the difference (OD 450-OD 530) calculated.

The TNFα ELISA was performed as follows. Anti-human TNFα antibody (Pierce Endogen, Rockford, Ill.; catalogue number M303-E, lot number 018334) was diluted to 2 μg/ml in phosphate buffered saline, pH 7.2-7.4, and 100 μl of the diluted antibody was added to each well of Nunc Maxisorb ELISA strip plates. The plates were incubated overnight at room temperature. The liquid was aspirated from the wells, and the plates were blotted on a paper towel. Then, 200 μl of assay buffer were added to each well, and the plates were incubated for 1 hour at room temperature. The liquid was aspirated from the wells, and the wells were washed 3 times with wash buffer, and the plates were then blotted on a paper towel. Standards and CGF samples (50 μl/well; standards were diluted in storage buffer) were added to the wells, and the plates were incubated for 1 hour at room temperature with gentle shaking. The liquid was aspirated, the wells were washed 3 times with wash buffer, and the plates were then blotted on a paper towel. Then, 100 μl of biotin-labeled anti-human TNFA (Pierce Endogen, Rockford, Ill.; catalogue number M302-B, lot number 017005), diluted to 250 ng/ml in assay buffer, were added to each well. The plates were incubated for 1 hour at room temperature, the liquid was aspirated, the wells were washed 3 times with wash buffer, and the plates were blotted on a paper towel. Then, 100 μl of HRP-conjugated streptavidin in assay buffer, were added to each well. The plates were incubated for 30 minutes at room temperature, the liquid was aspirated, the wells were washed 3 times with wash buffer, and the plates were blotted on a paper towel. Finally, 100 μl of TMB substrate solution were added to each well. The plates were incubated for 30 minutes at room temperature. The reaction was stopped by adding 100 μl/well of 0.18 M H₂SO₄. The optical densities at 450 nm and 530 nm were read on an ELISA plate reader and the difference (OD 450-OD 530) calculated.

The sTNFR75 ELISA assay was performed using the Quantikine sTNFR75 ELISA kit (R & D Systems, Minneapolis, Minn.) according to the manufacturer's instrictions. Briefly, 50 μl of each standard and of each CGF sample were added to the wells of plates coated with anti-sTNFR75 antibody, and the plates were incubated for 2 hours at room temperature. Then, the liquid was aspirated from each well, and the wells were washed three times with 400 μl/well of supplied wash buffer. Next, 200 μl sTNFR75 conjugate solution were added to each well, and the plates were incubated at room temperature for 1 hour. Again, the liquid was aspirated from each well and the wells were washed three times with 400 μl/well of supplied wash buffer. After washing was completed, 200 μl of the supplied substrate solution were added to each well, and the plates were incubated for 20 minutes at room temperature. Finally, 50 μl of stop solution were added to each well, and the optical density at 450 nm minus the optical density at 530 nm was determined.

With respect to the CGF samples taken without the application of Crest Whitestrips, the IL-8 ELISA was performed on all of the samples (samples from 11 normal controls, 9 periodontitis patients and 7 gingivitis patients), the TNFα ELISA was performed on CGF samples from 2 of the normal controls, 1 of the periodontitis patients and 3 of the gingivitis patients, and the sTNFR75 ELISA was performed on CGF samples from 1 normal control, 5 periodontitis patients, and 3 gingivitis patients. The IL-8 ELISA was performed on all of the CGF samples taken from the 5 normal controls who had the Crest Whitestrips™ applied to their teeth and on all of the CGF samples taken from the 3 normal controls to who had the Crest WhitestripS™ with DAHK 1199 applied to their teeth. All samples were assayed in duplicate.

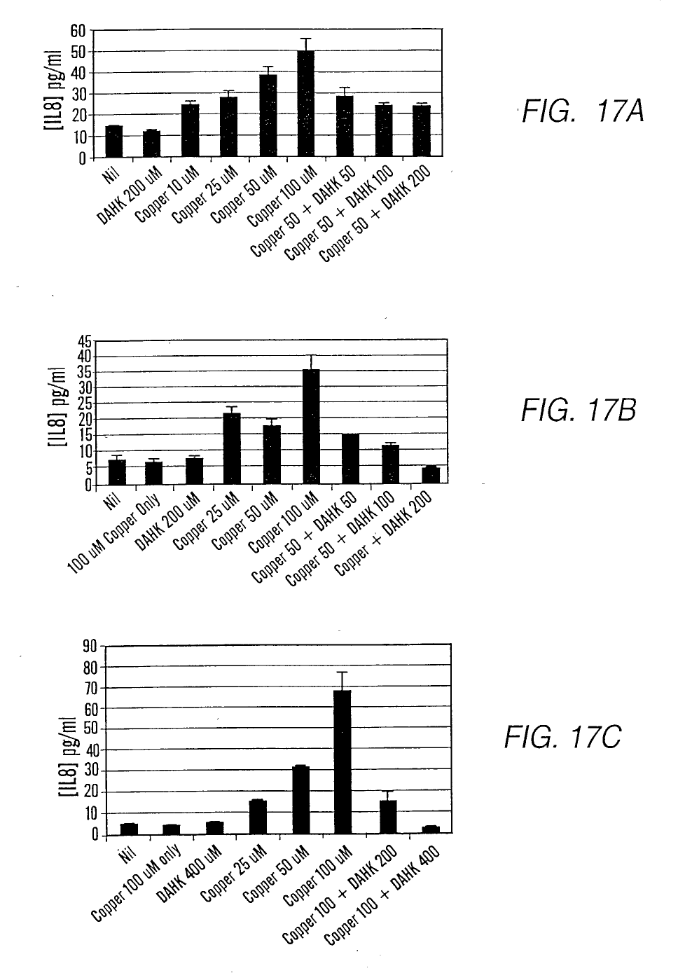

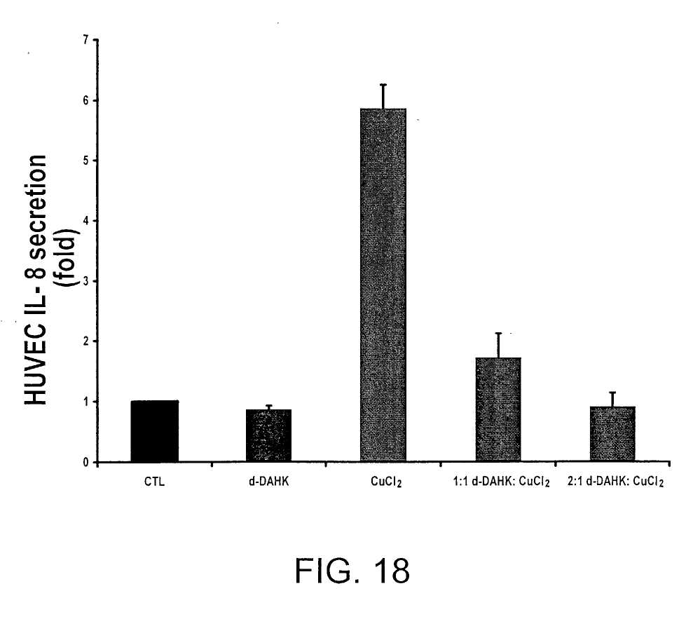

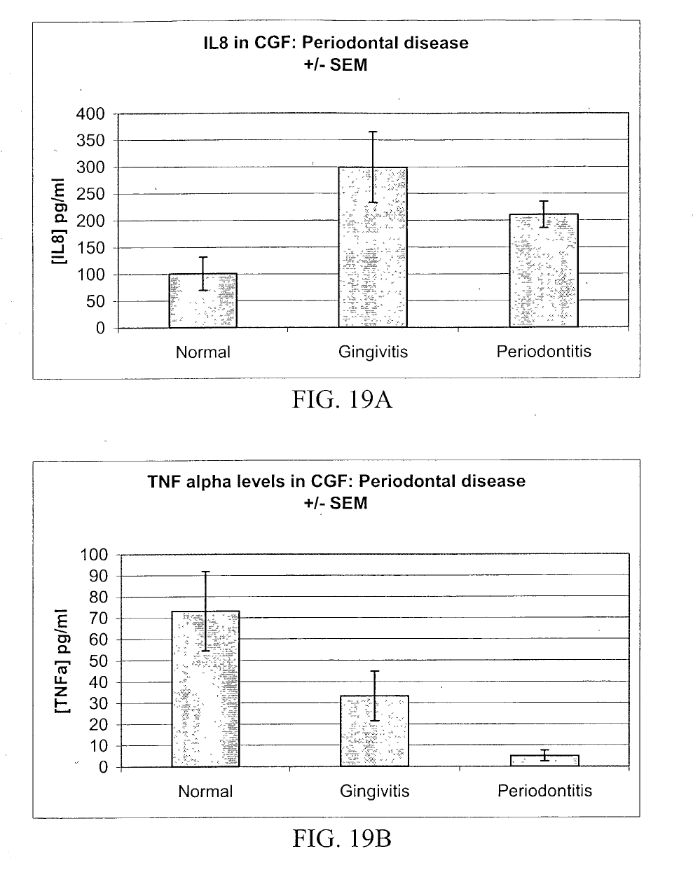

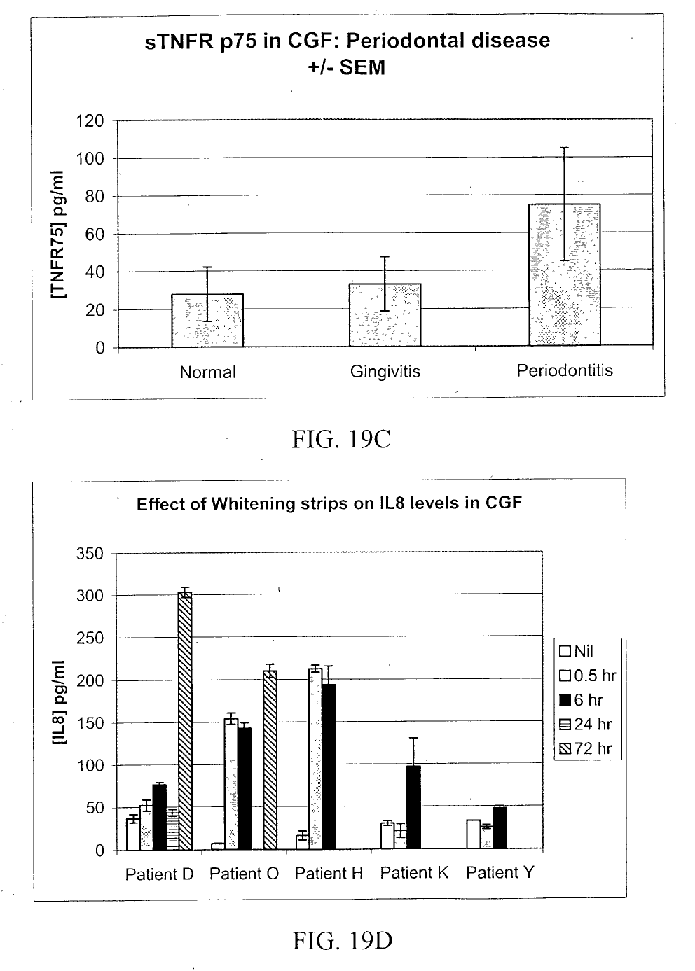

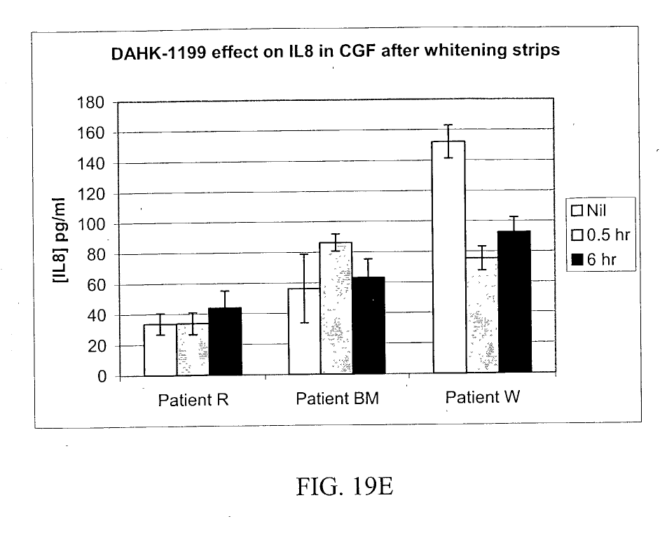

The results are presented in FIGS. 19A-E. As can be seen in FIGS. 19A-C, IL-8 and sTNFR75 were elevated, and TNFα was decreased in patients with gingivitis and periodontitis, as compared to normal controls. Treatment with Crest Whitestrips™ increased the amount of IL-8 in the CGF of the normal controls by six hours after treatment (see FIG. 19D). Treatment with Crest Whitestrips™ TM with DAHK-1199 reduced the amount of IL-8 in the CGF of the normal controls at six hours compared to Crest Whitestrips™ without DAHK-1 199 (compare FIG. 19E with FIG. 19D).

We claim:

1. A method of treating a tissue of an animal's mouth comprising contacting the tissue with an effective amount of a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

2. The method of claim 1 wherein the tissue is treated prophylactically.

3. The method of claim 2 wherein the tissue is treated as part of a prophylactic oral regimen.

4. The method of claim 1 wherein the tissue is treated prior to surgery, during surgery, after surgery, or combinations thereof.

5. The method of claim 1 wherein the tissue is treated prior to a tooth extraction, during a tooth extraction, after a tooth extraction, or combinations thereof.

6. The method of claim 1 wherein the tissue is all or substantially all of the tissue of the mouth.

7. A method of treating a disease or condition of a tissue of an animal's mouth comprising contacting the tissue with an effective amount of a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine,- methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

8. The method of claim 6 wherein the disease or condition is a disease or condition of the periodontal tissue.

9. The method of claim 7 wherein the disease or condition is gingivitis or periodontitis.

10. The method of claim 6 wherein the disease or condition is an infection.

11. A method oftreating inflammation of a tissue of an animal's mouth comprising contacting the tissue with an effective amount of a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

12. The method of claim 11 wherein the inflammation is inflammation of the periodontal tissue.

13. A method of whitening one or more teeth of an animal comprising contacting a tissue of the animal's mouth with an effective amount of a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

14. The method of claim 13 wherein the tissue is all or substantially all of the tissue of the mouth.

15. The method of claim 13 wherein the tissue is contacted with the peptide prior to whitening of the teeth, during whitening of the teeth, after whitening of the teeth, or combinations thereof.

16. A method of reducing the damage done by reactive oxygen species (ROS) to a tissue of an animal's mouth comprising contacting the tissue with an effective amount of a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, omithine, aspartic acid, glutamic acid, asparagine, glutamine or tryptophan;
Xaa₄ is any amino acid; and
n is 0-100;
or a physiologically-acceptable salt thereof.

17. A method of reducing the concentration of a metal in or on a tissue of an animal's mouth comprising contacting the tissue with an effective amount of apeptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

18. The method of any one of claims 1-17 wherein Xaa₁ is aspartic acid, glutamic acid, arginine, threonine, or α-hydroxymethylserine.

19. The method of any one of claims 1-17 wherein Xaa₂ is glycine, alanine, valine, leucine, isoleucine, threonine, serine, asparagine, methionine, histidine or α-hydroxymethylserine.

20. The method of any one of claims 1-17 wherein Xaa₃ is lysine.

21. The method of any one of claims 1-17 wherein Xaa₂ is aspartic acid, glutamic acid, arginine, threonine, or α-hydroxymethylserine, Xaa₂ is glycine, alanine, valine, leucine, isoleucine, threonine, serine, asparagine, methionine, histidine or α-hydroxymethylserine, and Xaa₃ is lysine.

22. The method of claim 21 wherein Xaa₁ is aspartic acid or glutamic acid and Xaa₂ is alanine, glycine, valine, threonine, serine, leucine, or α-hydroxymethylserine.

23. The method of claim 22 wherein Xaa₂ is alanine, threonine, leucine, or α-hydroxymethylserine.

24. The method of claim 23 wherein Xaa₁ is aspartic acid and Xaa₂ is alanine.

25. The method of any one of claims 1-17 wherein n is 0-10.

26. The method of claim 25 wherein n is 0-5.

27. The method of claim 26 wherein n is 0.

28. The method of any one of claims 1-17 wherein P₂ comprises a metal-binding sequence.

29. The method of claim 28 wherein P₂ comprises one of the following sequences:
(Xaa₄)ₘ Xaa₃ His Xaa₂ Xaa₅,
(Xaa₄)ₘ His Xaa₂ Xaa₅,
(Xaa₄)ₘ Xaa₅ Xaa₂ His Xaa₃, or
(Xaa₄)ₘ Xaa₅ Xaa₂ His,
wherein Xaa₅ is an amino acid having a free side-chain —NH₂ and m is 0-5.

30. The method of claim 29 wherein Xaa₅ is Orn or Lys.

31. The method of claim 28 wherein P₂ comprises one of the following sequences:
[(Xaa₄)ₘXaa₅Xaa₂HisXaa₃]ᵣ,
[(Xaa₄)ₘXaa₅Xaa₂His]ᵣ,
[(Xaa₄)ₘXaa₅Xaa₂HisXaa₃(Xaa₄)ₘXaa₅Xaa₂His]ᵣ, or
[(Xaa₄)ₘXaa₅Xaa₂His(Xaa₄)ₘXaa₅Xaa₂HisXaa₃]ᵣ,
wherein Xaa₅ is an amino acid having a free side-chain —NH₂, m is 0-5 and r is 2-100.

32. The method of claim 28 wherein P₂ comprises a sequence which binds Cu(I).

33. The method of claim 32 wherein P₂ comprises one of the following sequences:
Met Xaa₄ Met,
Met Xaa₄ Xaa₄ Met,
Cys Cys,
Cys Xaa₄ Cys,
Cys Xaa₄ Xaa₄ Cys,
Met Xaa₄ Cys Xaa₄ Xaa₄ Cys,
Gly Met Xaa₄ Cys Xaa₄ Xaa₄ Cys [SEQ ID NO:7],
Gly Met Thr Cys Xaa₄ Xaa₄ Cys [SEQ ID NO:8],
Gly Met Thr Cys Ala Asn Cys [SEQ ID NO:9], or
γ-Glu Cys Gly.

34. The method of claim 33 wherein P₂ is Gly Met Thr Cys Ala Asn Cys [SEQ ID NO:9].

35. The method of any one of claims 1-17 wherein at least one of the amino acids of P₁ other than β-alanine, when present, is a D-amino acid.

36. The method of claim 35 wherein Xaa₁ is a D-amino acid, His is a D-amino acid, or both Xaa₁ and His are D-amino acids.

37. The method of claim 36 wherein all of the amino acids of P₁ other than β-alanine, when present, are D-amino acids.

38. The method of claim 35 wherein at least 50% of the amino acids of P₂ are D-amino acids.

39. The method of claim 36 wherein at least 50% of the amino acids of P₂ are D-amino acids.

40. The method of claim 37 wherein at least 50% of the amino acids of P₂ are D-amino acids.

41. The method of any one of claims 1-17 wherein at least one amino acid of P₂, at least one amino acid of P₂, or at least one amino acid of P₁ and at least one amino acid of P₂, is substituted with (a) a substituent that increases the lipophilicity of the peptide without altering the ability of P₁ to bind metal ions, (b) a substituent that protects the peptide from proteolytic enzymes without altering the ability of P₁ to bind metal ions, or (c) a substituent which is a non-peptide, metal-binding functional group that improves the ability ofthe peptide to bind metal ions.

42. The method of claim 41 wherein n is 0 and P₁ has one of the following formulas: 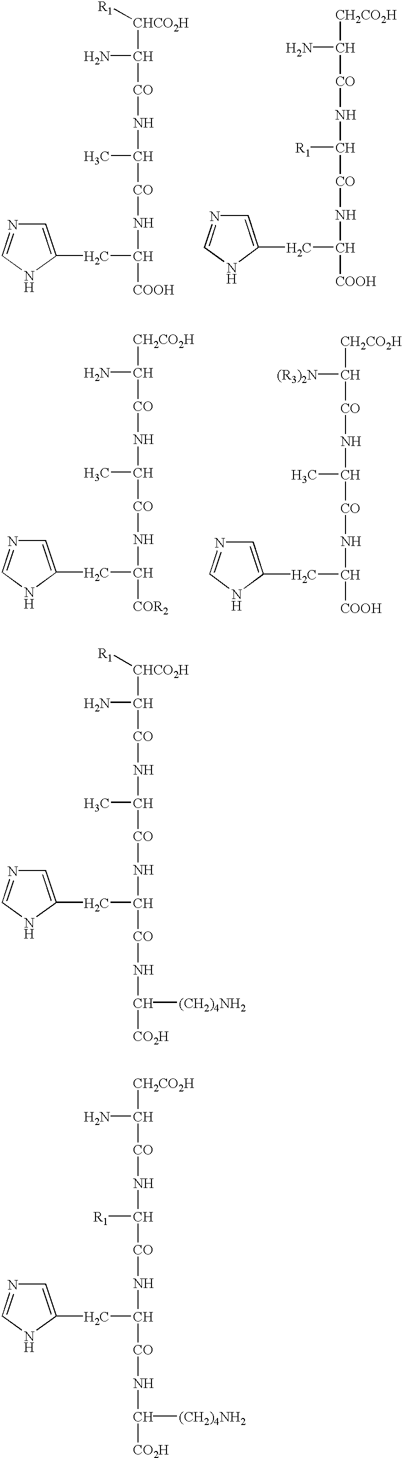 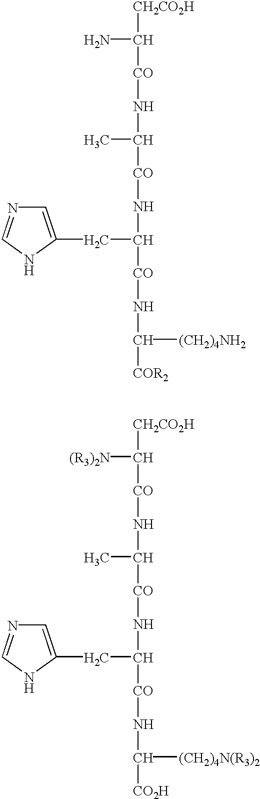
wherein:
R₁ is an alkyl, aryl, or heteroaryl;
R₂ is —NH₂, —NHR₁, N(R₁)₂, —OR₁, or R₁; and
R₃ is H, a non-peptide, metal-binding functional group or the two R₃ groups together form a non-peptide, metal-binding functional group.

43. The method of any one of claims 1-17 wherein the method further comprises contacting the tissue with an effective amount of another metal-binding compound in combination with the peptide.

44. The method of claim 43 wherein the metal-binding compound binds iron.

45. The method of claim 44 wherein the iron-binding compound is deferoxamine mesylate.

46. The method of claim 43 wherein the metal-binding compound binds Cu(I).

47. The method of claim 46 wherein the Cu(I)-binding compound is a peptide.

48. The method of claim 47 wherein the Cu(I)-binding peptide comprises one of the following sequences:
Met Xaa₄ Met,
Met Xaa₄ Xaa₄ Met,
Cys Cys
Cys Xaa₄ Cys,
Cys Xaa₄ Xaa₄ Cys,
Met Xaa₄ Cys Xaa₄ Xaa₄ Cys,
Gly Met Xaa₄ Cys Xaa₄ Xaa₄ Cys [SEQ ID NO:7],
Gly Met Thr Cys Xaa₄ Xaa₄ Cys [SEQ ID NO:8],
Gly Met Thr Cys Ala Asn Cys [SEQ ID NO:9], or
γy-Glu Cys Gly.

49. A method of treating a tissue of an animal's mouth comprising contacting the tissue with an effective amount of a metal-binding peptide having attached thereto a non-peptide, metal-binding functional group.

50. The method of claim 49 wherein the peptide contains from 2-10 amino acids.

51. The method of claim 50 wherein the peptide contains from 3-5 amino acids.

52. The method of claim 49 wherein the amino acids of the peptide are D-amino acids.

53. The method of claim 49 wherein the method further comprises contacting the tissue with an effective amount of a second metal-binding compound.

54. A method of treating a tissue of an animal's mouth comprising contacting the tissue with an effective amount of a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

55. A method of treating a disease or condition of a tissue of an animal's mouth comprising contacting the tissue with an effective amount of a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

56. A method oftreating inflammation of a tissue of an animal's mouth comprising contacting the tissue with an effective amount of a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

57. A method of whitening one or more teeth of an animal comprising contacting a tissue of the animal's mouth with an effective amount of a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

58. A method of reducing the damage done by reactive oxygen species (ROS) to a tissue of an animal's mouth comprising contacting the tissue with an effective amount of a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

59. A method of reducing the concentration of a metal in or on a tissue of an animal's mouth comprising contacting the tissue with an effective amount of a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

60. The method of any one of claims 54-59 wherein each P₃ contains 2-10 amino acids.

61. The method of any one of claims 54-59 wherein at least one P₃ is P₁, wherein P₁ is:
Xaa₁ Xaa₂ His: or
Xaa, Xaa₂ His Xaa₃; and
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine; and
Xaa₃ is glycine, alanine, valine, lysine, arginine, ornithine, aspartic acid, glutamic acid, asparagine, glutamine or tryptophan.

62. The method of claim 61 wherein Xaa₁ is aspartic acid, glutamic acid, arginine, threonine, or α-hydroxymethylserine.

63. The method of claim 61 wherein Xaa₂ is glycine, alanine, valine, leucine, isoleucine, threonine, serine, asparagine, methionine, histidine or α-hydroxymethylserine.

64. The method of claim 61 wherein Xaa₃ is lysine.

65. The method of claim 61 wherein Xaa₁ is aspartic acid, glutamic acid, arginine, threonine, or α-hydroxymethylserine, Xaa₂ is glycine, alanine, valine, leucine, isoleucine, threonine, serine, asparagine, methionine, histidine or α-hydroxymethylserine, and Xaa₃ is lysine.

66. The method of claim 65 wherein Xaa₁ is aspartic acid or glutamic acid and Xaa₂ is alanine, glycine, valine, threonine, serine, leucine, or α-hydroxymethylserine.

67. The method of claim 66 wherein Xaa₂ is alanine, threonine, leucine, or α-hydroxymethylserine.

68. The method of claim 67 wherein Xaa₁ is aspartic acid and Xaa₂ is alanine.

69. The method of claim 61 wherein at least one amino acid of P₁ other than β-alanine, when present, is a D-amino acid.

70. The method of claim 69 wherein all of the amino acids of P₁ other than β-alanine, when present, are D-amino acids.

71. The method of claim 61 wherein both P₃ peptides are P₁.

72. The method of any one of claims 54-59 wherein at least one amino acid of P₃ is substituted with (a) a substituent that increases the lipophilicity of the peptide dimer without altering the ability of P₃ to bind metal ions, (b) a substituent that protects the peptide dimer from proteolytic enzymes without altering the ability of P₃ to bind metal ions, or (c) a substituent which is a non-peptide, metal-binding functional group that improves the ability of the peptide dimer to bind metal ions.

73. The method of any one of claims 54-59 wherein P₃ comprises an amino acid sequence which is substituted with a non-peptide, metal-binding functional group to provide the metal-binding capability of P₃.

74. The method of any one of claims 54-59 wherein L is neutral.

75. The method of any one of claims 54-59 wherein L is a straight-chain or branched-chain alkane or alkene residue containing from 1-18 carbon atoms.

76. The method of claim 75 wherein L contains 2-8 carbon atoms.

77. The method of any one of claims 54-59 wherein L is a cyclic alkane residue containing from 2-8 carbon atoms.

78. The method of claim 77 wherein L contains 3-5 carbon atoms.

79. The method of any one of claims 54-59 wherein L is a nitrogen-containing heterocyclic alkane residue.

80. The method of claim 79 wherein L is a piperazide.

81. The method of any one of claims 54-59 wherein L is a glyceryl ester.

82. The method of any one of claims 54-59 wherein the method further comprises contacting the tissue with an effective amount of another metal-binding compound in combination with the peptide dimer.

83. An oral care product comprising a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

84. An oral care device comprising a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, omithine, aspartic acid, glutamic acid, asparagine, glutamine or tryptophan;
Xaa₄ is any amino acid; and
n is 0-100;
or a physiologically-acceptable salt thereof.

85. The device of claim 84 which is a suture or a dental floss.

86. The device of claim 84 which is a strip.

87. The device of claim 86 wherein the strip further comprises a tooth whitening agent.

88. An oral care composition comprising a pharmaceutically-acceptable carrier and a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, ornithine, aspartic acid, glutamic acid, asparagine, glutamine or tryptophan;
Xaa₄ is any amino acid; and
n is 0-100;
or a physiologically-acceptable salt thereof.

89. The composition of claim 88 wherein the composition is a gel, a paste, an ointment, a cream, a powder, a wash, a rinse, a gargle, a spray, a solution, a tablet, a gum, a lozenge, a mint, a film, or a patch.

90. The composition of claim 88 wherein the composition is a tooth whitening composition.

91. The peptide of any one of claims 83-90 wherein Xaa₁ is aspartic acid, glutamic acid, arginine, threonine, or α-hydroxymethylserine.

92. The peptide of any one of claims 83-90 wherein Xaa₂ is glycine, alanine, valine, leucine, isoleucine, threonine, serine, asparagine, methionine, histidine or α-hydroxymethylserine.

93. The peptide of any one of claims 83-90 wherein Xaa₃ is lysine.

94. The peptide of any one of claims 83-90 wherein Xaa₁ is aspartic acid, glutamic acid, arginine, threonine, or α-hydroxymethylserine, Xaa₂ is glycine, alanine, valine, leucine, isoleucine, threonine, serine, asparagine, methionine, histidine or α-hydroxymethylserine, and Xaa₃ is lysine.

95. The peptide of claim 94 wherein Xaa₁ is aspartic acid or glutamic acid and Xaa₂ is alanine, glycine, valine, threonine, serine, leucine, or α-hydroxymethylserine.

96. The peptide of claim 95 wherein Xaa₂ is alanine, threonine, leucine, or α-hydroxymethylserine.

97. The peptide of claim 96 wherein Xaa₁ is aspartic acid and Xaa₂ is alanine.

98. The peptide of any one of claims 83-90 wherein n is 0-10.

99. The peptide of claim 98 wherein n is 0-5.

100. The peptide of claim 99 wherein n is 0.

101. The peptide of any one of claims 83-90 wherein P₂ comprises ametal-binding sequence.

102. Thepeptide of claim 101 wherein P₂ comprises one of the following sequences:
(Xaa₄)ₘ Xaa₃ His Xaa₂ Xaa₅,
(Xaa₄)ₘ His Xaa₂ Xaa₅,
(Xaa₄)ₘ Xaa₅ Xaa₂ His Xaa₃, or
(Xaa₄)ₘ Xaa₅ Xaa₂ His,
wherein Xaa₅ is an amino acid having a free side-chain —NH₂ and m is 0-5.

103. The peptide of claim 102 wherein Xaa₅ is Orn or Lys.

104. The peptide of claim 101 wherein P₂ comprises one of the following sequences:
[(Xaa₄)ₘXaa₅Xaa₂HisXaa₃]ᵣ,
[(Xaa₄)ₘXaa₅Xaa₂His]ᵣ,
[(Xaa₄)ₘXaa₅Xaa₂HisXaa₃(Xaa₄)ₘXaa₅Xaa₂His]ᵣ, or
[(Xaa₄)ₘXaa₅Xaa₂His(Xaa₄)ₘXaa₅Xaa₂HisXaa₃]ᵣ,
wherein Xaa₅ is an amino acid having a free side-chain —NH₂, m is 0-5 and r is 2-100.

105. The peptide of claim 101 wherein P₂ comprises a sequence which binds Cu(I).

106. The peptide of claim 105 wherein P₂ comprises one of the following sequences:
Met Xaa₄ Met,
Met Xaa₄ Xaa₄ Met,
Cys Cys,
Cys Xaa₄ Cys,
Cys Xaa₄ Xaa₄ Cys,
Met Xaa₄ Cys Xaa₄ Xaa₄ Cys,
Gly Met Xaa₄ Cys Xaa₄ Xaa₄ Cys [SEQ ID NO:7],
Gly Met Thr Cys Xaa₄ Xaa₄ Cys [SEQ ID NO:8],
Gly Met Thr Cys Ala Asn Cys [SEQ ID NO:9], or
γ-Glu Cys Gly.

107. The peptide of claim 106 wherein P₂ is Gly Met Thr Cys Ala Asn Cys [SEQ ID NO:9].

108. The peptide of any one of claims 83-90 wherein P₂ comprises a sequence which enhances the ability of the peptide to penetrate cell membranes.

109. The peptide of claim 108 wherein P₂ is hydrophobic or an arginine oligomer.

110. The peptide of any one of claims 83-90 wherein at least one of the amino acids of P₁ other than β-alanine, when present, is a D-amino acid.

111. The peptide of claim 110 wherein Xaa₁ is a D-amino acid, His is a D-amino acid, or both Xaaᵢ and His are D-amino acids.

112. The peptide of claim 111 wherein all of the amino acids of P₁ other than β-alanine, when present, are D-amino acids.

113. The peptide of claim 110 wherein at least 50% of the amino acids of P₂ are D-amino acids.

114. The peptide of claim 111 wherein at least 50% of the amino acids of P₂ are D-amino acids.

115. The peptide of claim 112 wherein at least 50% of the amino acids of P₂ are D-amino acids.

116. The peptide of any one of claims 83-90 wherein at least one amino acid of P₁, at least one amino acid of P₂, or at least one amino acid of P₁ and at least one amino acid of P₂ is substituted with (a) a substituent that increases the lipophilicity of the peptide without altering the ability of P₁ to bind metal ions, (b) a substituent that protects the peptide from proteolytic enzymes without altering the ability of P₁ to bind metal ions, or (c) a substituent which is a non-peptide, metal-binding functional group that increases the ability of the peptide to bind metal ions.

117. The peptide of claim 116 wherein n is 0 and P₁ has one of the following formulas: 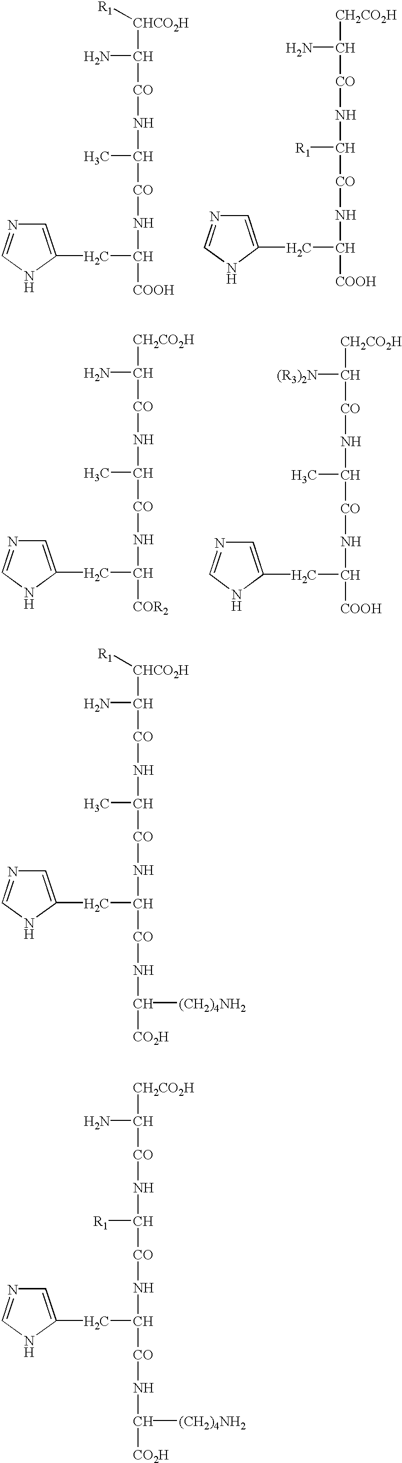 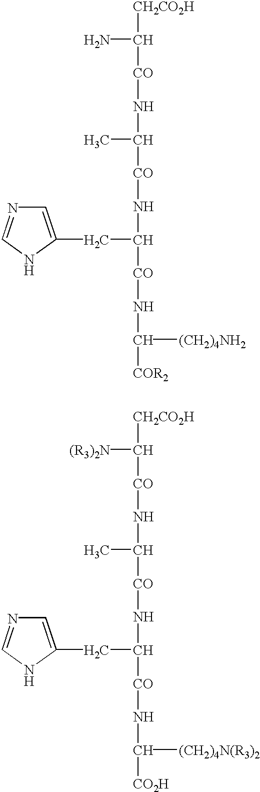
wherein:
R₁ is an alkyl, aryl, or heteroaryl;
R₂ is —NH₂, —NHR₁, N(R₁)₂, —OR₁, or R₁; and
R₃ is H, a non-peptide, metal-binding functional group or the two R₃ groups together form a non-peptide, metal-binding functional group.

119. An oral care product comprising a metal-binding peptide having attached thereto a non-peptide, metal-binding functional group.

120. The product of claim 119 wherein the peptide contains from 2-10 amino acids.

121. The product of claim 120 wherein the peptide contains from 3-5 amino acids.

122. The product of claim 119 wherein the amino acids of the peptide are D-amino acids.

123. The product of claim 119 further comprising a second metal-binding compound.

124. An oral care product comprising a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

125. An oral care device comprising a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

126. An oral care composition comprising a pharmaceutically-acceptable carrier and a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

127. The peptide dimer of any one of claims 124-126 wherein each P₃ contains 2-10 amino acids.

128. The peptide dimer of any one of claims 124-126 wherein at least one P₃iS P₁ wherein P₁ is:
Xaa₁ Xaa₂ His: or
Xaa₁ Xaa₂ His Xaa₃; and
Xaa, is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine; and
Xaa₃ is glycine, alanine, valine, lysine, arginine, ornithine, aspartic acid, glutamic acid, asparagine, glutamine or tryptophan.

129. The peptide dimer of claim 128 wherein Xaa₁ is aspartic acid, glutamic acid, arginine, threonine, or α-hydroxymethylserine.

130. The peptide dimer of claim 128 wherein Xaa₂ is glycine, alanine, valine, leucine, isoleucine, threonine, serine, asparagine, methionine, histidine or α-hydroxymethylserine.

131. The peptide dimer of claim 128 wherein Xaa₃ is lysine.

132. The peptide dimer of claim 128 wherein Xaa₁ is aspartic acid, glutamic acid, arginine, threonine, or α-hydroxymethylserine, Xaa2 is glycine, alanine, valine, leucine, isoleucine, threonine, serine, asparagine, methionine, histidine or α-hydroxymethylserine, and Xaa₃ is lysine.

133. The peptide dimer of claim 132 wherein Xaa₁ is aspartic acid or glutamic acid and Xaa₂ is alanine, glycine, valine, threonine, serine, leucine, or α-hydroxymethylserine.

134. The peptide dimer of claim 133 wherein Xaa₂ is alanine, threonine, leucine, or α-hydroxymethylserine.

135. The peptide dimer of claim 134 wherein Xaa₁ is aspartic acid and Xaa₂ is alanine.

136. The peptide dimer of claim 128 wherein at least one amino acid of P₁ other than β-alanine, when present, is a D-amino acid.

137. The peptide dimer of claim 136 wherein all of the amino acids of P₁ other than β-alanine, when present, are D-amino acids.

138. The peptide dimer of claim 128 wherein both P₃peptides are P₁.

139. The peptide dimer of any one of claims 124-126 wherein at least one amino acid of P₃ is substituted with (a) a substituent that increases the lipophilicity of the peptide dimer without altering the ability of P₃ to bind metal ions, (b) a substituent that protects the peptide dimer from proteolytic enzymes without altering the ability of P₃ to bind metal ions, or (c) a substituent which is a non-peptide, metal-binding functional group that improves the ability of the peptide dimer to bind metal ions.

140. The peptide dimer of any one of claims 124-126 wherein P₃ comprises an amino acid sequence which is substituted with a non-peptide, metal-binding functional group to provide the metal-binding capability of P₃.

141. The peptide dimer of any one of claims 124-126 wherein L is neutral.

142. The peptide dimer of any one of claims 124-126 wherein L is a straight-chain or branched-chain alkane or alkene residue containing from 1-18 carbon atoms.

143. The peptide dimer of claim 142 wherein L contains 2-8 carbon atoms.

144. The peptide dimer of any one of claims 124-126 wherein L is a cyclic alkane residue containing from 2-8 carbon atoms.

145. The peptide dimer of claim 144 wherein L contains 3-5 carbon atoms.

146. The peptide dimer of any one of claims 124-126 wherein L is a nitrogen-containing heterocyclic alkane residue.

147. The peptide dimer of claim 146 wherein L is a piperazide.

148. The peptide dimer of any one of claims 124-126 wherein L is a glyceryl ester.

149. A kit comprising an oral care product, the oral care product comprising a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, omithine, aspartic acid, glutamic acid, asparagine, glutamine or tryptophan;
Xaa₄ is any amino acid; and
n is 0-100;
or a physiologically-acceptable salt thereof.

150. A kit comprising an oral care device, the oral care device comprising a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, ornithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, omithine, aspartic acid, glutamic acid, asparagine, glutamine or tryptophan;
Xaa₄ is any amino acid; and
n is 0-100;
or a physiologically-acceptable salt thereof

151. The kit of claim 150 wherein the device is a strip.

152. The kit of claim 151 wherein the strip further comprises a tooth whitening agent.

153. The kit of claim 151 wherein the kit further comprises a tooth whitening composition.

154. The kit of claim 151 wherein the kit further comprises a second strip, the second strip comprising a tooth whitening agent.

155. A kit comprising an oral care composition, the composition comprising a pharmaceutically-acceptable carrier and a peptide having the formula:
P₁-P₂,
wherein:
P₁ is:
Xaa₁ Xaa₂ His: or
Xaa₁ Xaa₂ His Xaa₃;
P₂ is (Xaa₄)ₙ;
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine;
Xaa₃ is glycine, alanine, valine, lysine, arginine, omithine, aspartic acid, glutamic acid, asparagine, glutamine or tryptophan;
Xaa₄ is any amino acid; and
n is 0-100;
or a physiologically-acceptable salt thereof.

156. The kit of claim 155 where the composition is a tooth whitening composition.

157. The kit of claim 156 wherein the composition is a gel.

158. The kit of claim 155 wherein the kit further comprises a strip comprising a tooth whitening agent.

159. The kit of any one of claims 149-158 wherein Xaa₁ is aspartic acid, glutamic acid, arginine, threonine, or α-hydroxymethylserine.

160. The kit of any one of claims 149-158 wherein Xaa₂ is glycine, alanine, valine, leucine, isoleucine, threonine, serine, asparagine, methionine, histidine or α-hydroxymethylserine.

162. The kit of any one of claims 149-158 wherein Xaa₃ is lysine.

163. The kit of any one of claims 149-158 wherein Xaa₁ is aspartic acid, glutamic acid, arginine, threonine, or α-hydroxymethylserine, Xaa₂ is glycine, alanine, valine, leucine, isoleucine, threonine, serine, asparagine, methionine, histidine or α-hydroxymethylserine, and Xaa₃ is lysine.

164. The kit of claim 163 wherein Xaa₁ is aspartic acid or glutamic acid and Xaa₂ is alanine, glycine, valine, threonine, serine, leucine, or α-hydroxymethylserine.

165. The kit of claim 164 wherein Xaa₂ is alanine, threonine, leucine, or α-hydroxymethylserine.

166. The kit of claim 165 wherein Xaa₁ is aspartic acid and Xaa₂ is alanine.

167. The kit of any one of claims 149-158 wherein n is 0-10.

168. The kit of claim 167 wherein n is 0-5.

169. The kit of claim 168 wherein n is 0.

170. The kit of any one of claims 149-158 wherein P₂ comprises a metal-binding sequence.

171. The kit of claim 170 wherein P₂ comprises one of the following sequences:
(Xaa₄)ₘ Xaa₃ His Xaa₂ Xaa₅,
(Xaa₄)ₘ His Xaa₂ Xaa₅,
(Xaa₄)ₘ Xaa₅ Xaa₂ His Xaa₃, or
(Xaa₄)ₘ Xaa₅ Xaa₂ His,
wherein Xaa₅ is an amino acid having a free side-chain —NH₂ and m is 0-5.

172. The kit of claim 171 wherein Xaa₅ is Orn or Lys.

173. The kit of claim 170 wherein P₂ comprises one of the following sequences:
[(Xaa₄)ₘXaa₅Xaa₂HisXaa₃]ᵣ,
[(Xaa₄)ₘXaa₅Xaa₂His]ᵣ,
[(Xaa₄)ₘXaa₅Xaa₂HisXaa₃(Xaa₄)ₘXaa₅Xaa₂His]ᵣ, or
[(Xaa₄)ₘXaa5Xaa₂His(Xaa₄)ₘXaa₅Xaa₂HisXaa₃]ᵣ,
wherein Xaa₅ is an amino acid having a free side-chain —NH₂, m is 0-5 and r is 2-100.

174. The kit of claim 170 wherein P₂ comprises a sequence which binds Cu(I).

175. The kit of claim 174 wherein P₂ comprises one of the following sequences:
Met Xaa₄ Met,
Met Xaa₄ Xaa₄ Met,
Cys Cys,
Cys Xaa₄ Cys,
Cys Xaa₄ Xaa₄ Cys,
Met Xaa₄ Cys Xaa₄ Xaa₄ Cys,
Gly Met Xaa₄ Cys Xaa₄ Xaa₄ Cys [SEQ ID NO:7],
Gly Met Thr Cys Xaa₄ Xaa₄ Cys [SEQ ID NO:8],
Gly Met Thr Cys Ala Asn Cys [SEQ ID NO:9], or
γ-Glu Cys Gly.

176. The kit of claim 175 wherein P₂ is Gly Met Thr Cys Ala Asn Cys [SEQ ID NO:9].

177. The kit of any one of claims 149-158 wherein P₂ comprises a sequence which enhances the ability of the peptide to penetrate cell membranes.

178. The kit of claim 177 wherein P₂ is hydrophobic or an arginine oligomer.

179. The kit of any one of claims 149-158 wherein at least one of the amino acids of P₁ other than β-alanine, when present, is a D-amino acid.

180. The kit of claim 179 wherein Xaa₁ is a D-amino acid, His is a D-amino acid, or both Xaa₁ and His are D-amino acids.

181. The kit of claim 180 wherein all of the amino acids of P₁ other than β-alanine, when present, are D-amino acids.

182. The kit of claim 179 wherein at least 50% of the amino acids of P₂ are D-amino acids.

183. The kit of claim 180 wherein at least 50% of the amino acids of P₂ are D-amino acids.

184. The kit of claim 181 wherein at least 50% of the amino acids of P₂ are D-amino acids.

185. The kit of any one of claims 149-158 wherein at least one amino acid of P₁, at least one amino acid of P₂, or at least one amino acid of P₁ and at least one amino acid of P₂ is substituted with (a) a substituent that increases the lipophilicity of the peptide without altering the ability of P₁ to bind metal ions, (b) a substituent that protects the peptide from proteolytic enzymes without altering the ability of P₁ to bind metal ions, or (c) a substituent which is a non-peptide, metal-binding functional group that increases the ability of the peptide to bind metal ions.

186. The kit of claim 185 wherein n is 0 and P₁ has one of the following formulas: 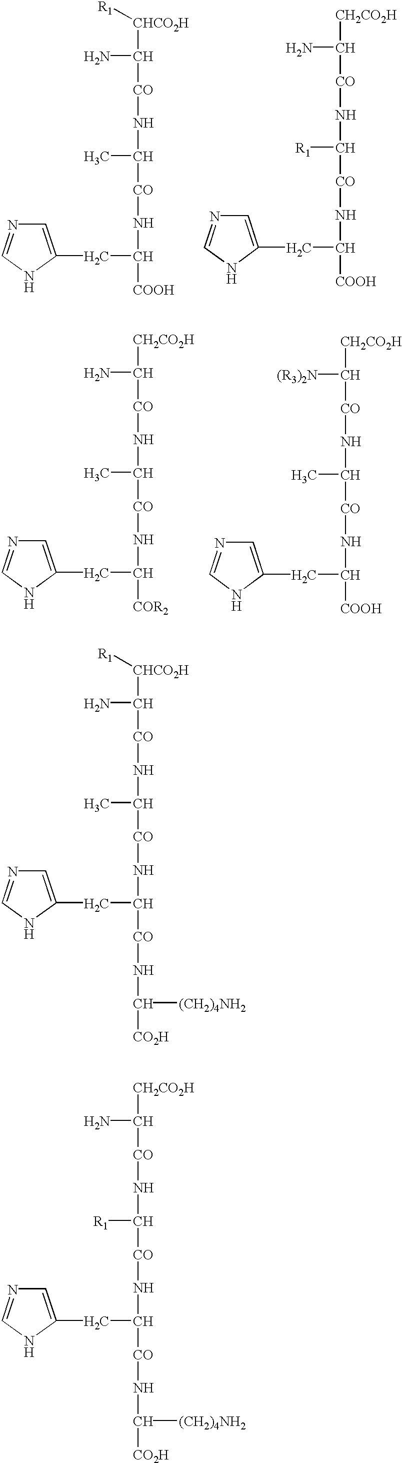 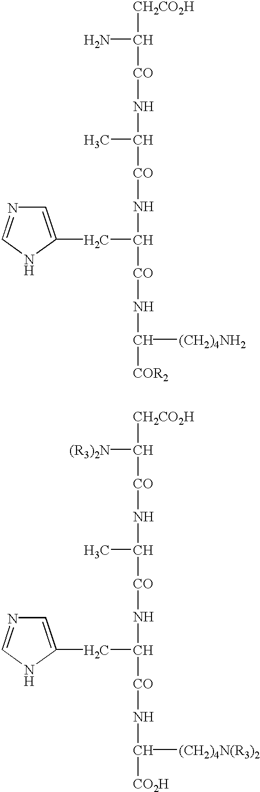
wherein:
R₁ is an alkyl, aryl, or heteroaryl;
R₂ is —NH₂, —NHR₁, N(R₁)₂, —OR₁, or R₁; and
R₃ is H, a non-peptide, metal-binding functional group or the two R₃ groups together form a non-peptide, metal-binding functional group.

187. A kit comprising an oral care product, the product comprising a metal-binding peptide having attached thereto a non-peptide, metal-binding functional group.

188. The kit of claim 187 wherein the peptide contains from 2-10 amino acids.

189. The kit of claim 188 wherein the peptide contains from 3-5 amino acids.

190. The kit of claim 187 wherein the amino acids of the peptide are D-amino acids.

191. The kit of claim 187 further comprising a second metal-binding compound.

192. A kit comprising an oral care product, the product comprising a metal-binding peptide dimer of the formula:
P₃-L- P₃,
wherein:
each P₃ may be the same or different and is a peptide which is capable of binding a metal ion; and
L is a chemical group which connects the two P₃ peptides through their C-terminal amino acids.

193. A kit comprising an oral care device, the device comprising a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

194. A kit comprising an oral care composition, the composition comprising a pharmaceutically-acceptable carrier and a metal-binding peptide dimer of the formula:
P₃-L-P₃,
wherein:
each P₃ may be the same or different and is a peptide which is capable of binding a metal ion; and
L is a chemical group which connects the two P₃ peptides through their C-terminal amino acids.

195. The kit of any one of claims 192-194 wherein each P₃contains 2-10 amino acids.

196. The kit of any one of claims 192-194 wherein at least one P₃ is P₁, wherein P₁ is:
Xaa, Xaa₂ His: or
Xaa, Xaa₂ His Xaa₃; and
Xaa₁ is glycine, alanine, valine, leucine, isoleucine, serine, threonine, aspartic acid, isoaspartic acid, asparagine, glutamic acid, isoglutamic acid, glutamine, lysine, hydroxylysine, histidine, arginine, omithine, phenylalanine, tyrosine, tryptophan, cysteine, methionine, or α-hydroxymethylserine;
Xaa₂ is glycine, alanine, β-alanine, valine, leucine, isoleucine, serine, threonine, aspartic acid, asparagine, glutamic acid, glutamine, lysine, hydroxylysine, histidine, arginine, ornithine, phenylalanine, tyrosine, tryptophan, cysteine, methionine, or α-hydroxymethylserine; and
Xaa₃ is glycine, alanine, valine, lysine, arginine, ornithine, aspartic acid, glutamic acid, asparagine, glutamine or tryptophan.

197. The kit of claim 196 wherein Xaa₁ is aspartic acid, glutamic acid, arginine, threonine, or α-hydroxymethylserine.

198. The kit of claim 196 wherein Xaa₂ is glycine, alanine, valine, leucine, isoleucine, threonine, serine, asparagine, methionine, histidine or α-hydroxymethylserine.

199. The kit of claim 196 wherein Xaa₃ is lysine.

200. The kit of claim 196 wherein Xaa₂ is aspartic acid, glutamic acid, arginine, threonine, or α-hydroxymethylserine, Xaa₂ is glycine, alanine, valine, leucine, isoleucine, threonine, serine, asparagine, methionine, histidine or α-hydroxymethylserine, and Xaa₃ is lysine.

201. The kit of claim 200 wherein Xaa₁ is aspartic acid or glutamic acid and Xaa₂ is alanine, glycine, valine, threonine, serine, leucine, or α-hydroxymethylserine.

202. The kit of claim 201 wherein Xaa₂ is alanine, threonine, leucine, or α-hydroxymethylserine.

203. The kit of claim 202 wherein Xaa₁ is aspartic acid and Xaa₂ is alanine.

204. The kit of claim 196 wherein at least one amino acid of P₁ other than β-alanine, when present, is a D-amino acid.

205. The kit of claim 204 wherein all of the amino acids of P₁ other than β-alanine, when present, are D-amino acids.

206. The kit of claim 196 wherein both P₃ peptides are P₁.

207. The kit of any one of claims 192-194 wherein at least one amino acid of P₃ is substituted with (a) a substituent that increases the lipophilicity of the peptide dimer without altering the ability of P₃ to bind metal ions, (b) a substituent that protects the peptide dimer from proteolytic enzymes without altering the ability of P₃ to bind metal ions, or (c) a substituent which is a non-peptide, metal-binding functional group that improves the ability of the peptide dimer to bind metal ions.

208. The kit of any one of claims 192-194 wherein P₃ comprises an amino acid sequence which is substituted with a non-peptide, metal-binding functional group to provide the metal-binding capability of P₃.

209. The kit of any one of claims 192-194 wherein L is neutral.

210. The kit of any one of claims 192-194 wherein L is a straight-chain or branched-chain alkane or alkene residue containing from 1-18 carbon atoms.

211. The kit of claim 210 wherein L contains 2-8 carbon atoms.

212. The kit of any one of claims 192-194 wherein L is a cyclic alkane residue containing from 2-8 carbon atoms.

213. The kit of claim 212 wherein L contains 3-5 carbon atoms.

214. The kit of any one of claims 192-194 wherein L is a nitrogen-containing heterocyclic alkane residue.

215. The kit of claim 214 wherein L is a piperazide.

216. The kit of any one of claims 192-194 wherein L is a glyceryl ester.
